(19) United States
(12) Patent Application Publication
(10) Pub. No.: US 2012/0207778 A1
(43) Pub. Date: Aug. 16, 2012

(54) NUCLEIC ACIDS AND PROTEINS FROM STREPTOCOCCUS GROUPS A AND B

(75) Inventors: John Telford, Siena (IT); Vega Masignani, Siena (IT); Immaculada Margarit Y Ros, Siena (IT); Guido Grandi, Siena (IT); Claire Fraser, Rockville, MD (US); Hervé Tettelin, Rockville, MD (US)

(73) Assignees: J. CRAIG VENTER INSTITUTE, INC., Rockville, MD (US); NOVARTIS VACCINES AND DIAGNOSTICS S.R.L., Siena (IT)

(21) Appl. No.: 13/398,079

(22) Filed: Feb. 16, 2012

Related U.S. Application Data
(62) Division of application No. 10/513,024, filed on Dec. 5, 2005, now abandoned.

(30) Foreign Application Priority Data
May 2, 2002 (GB) 0210128.5

(51) Int. Cl. A61K 39/09; C07K 16/12; C07K 7/08; A61K 38/16; A61P 31/04; C07K 7/06; C07K 14/315; C07H 21/04

(52) U.S. Cl. 424/190.1; 530/350; 530/387.9; 536/23.7; 514/2.6; 530/329; 530/328; 530/327; 530/326; 530/325; 530/324

(57) ABSTRACT

The invention provides proteins from group B streptococcus (Streptococcus agalactiae) and group A streptococcus (Streptococcus pyogenes), including amino acid sequences and the corresponding nucleotide sequences.

All documents cited herein are incorporated by reference in their entirety.

This application incorporates by reference a 5.29 MB text file created on Feb. 16, 2012 and named “PAT051229 sequencelisting.txt,” which is the sequence listing for this application.

### TECHNICAL FIELD

This invention relates to nucleic acid and proteins from the bacteria Streptococcus agalactiae (GBS) and Streptococcus pyogenes (GAS).

### BACKGROUND ART

Once thought to infect only cows, the Gram-positive bacterium Streptococcus agalactiae (or “group B streptococcus”, abbreviated to “GBS”) is now known to cause serious disease, bacteremia and meningitis, in immunocompromised individuals and in neonates. There are two types of neonatal infection. The first (early onset, usually within 5 days of birth) is manifested by bacteremia and pneumonia. It is contracted vertically as a baby passes through the birth canal. GBS colonises the vagina of about 25% of young women, and approximately 1% of infants born via a vaginal birth to colonised mothers will become infected. Mortality is between 50-70%. The second is a meningitis that occurs 10 to 60 days after birth. If pregnant women are vaccinated with type III capsule so that the infants are passively immunised, the incidence of the late onset meningitis is reduced but is not entirely eliminated.

The “B” in “GBS” refers to the Lancefield classification, which is based on the antigenicity of a carbohydrate which is soluble in dilute acid and called the C carbohydrate. Lancefield identified 13 types of C carbohydrate, designated A to 0, that could be serologically differentiated. The organisms that most commonly infect humans are found in groups A, B, D, and G. Within group B, strains can be divided into 8 serotypes (Ia, Ib, Ia/c, II, III, IV, V, and VI) based on the structure of their polysaccharide capsule.

Group A streptococcus (“GAS”, S. pyogenes) is a frequent human pathogen, estimated to be present in between 5-15% of normal individuals without signs of disease. When host defences are compromised, or when the organism is able to exert its virulence, or when it is introduced to vulnerable tissues or hosts, however, an acute infection occurs. Diseases include puerperal fever, scarlet fever, erysipelas, pharyngitis, impetigo, necrotising fasciitis, myositis and streptococcal toxic shock syndrome.

A genome sequence [accession AE004092] for GAS was published in 2001 [Ferretti et al. (2001) PNAS 98:4658-4663].

S. pyogenes is typically treated using antibiotics. Although S. agalactiae is inhibited by antibiotics, however, it is not killed by penicillin as easily as GAS. Prophylactic vaccination is thus preferable.

Current GBS vaccines are based on polysaccharide antigens, although these suffer from poor immunogenicity. Anti-idiotypic approaches have also been used (e.g. WO99/54457). There remains a need, however, for effective adult vaccines against S. agalactiae infection. There also remains a need for vaccines against S. pyogenes infection.

International patent application WO02/34771 discloses a large number of proteins and nucleic acids from GAS and GBS with the object of providing proteins which can be used in the development of vaccines, for diagnostic purposes, and as targets for antibiotics. It is an object of the present invention to provide further proteins which can be used in the development of vaccines, which can be used for diagnostic purposes, and which are targets for antibiotics.

### DISCLOSURE OF THE INVENTION

The invention provides proteins comprising the S. agalactiae amino acid sequences disclosed in the examples, and proteins comprising the S. pyogenes amino acid sequences disclosed in the examples. These amino acid sequences are the even-numbered SEQ IDs between 1 and 1372.

It also provides proteins comprising amino acid sequences having sequence identity to the S. agalactiae amino acid sequences disclosed in the examples, and proteins comprising amino acid sequences having sequence identity to the S. pyogenes amino acid sequences disclosed in the examples (in particular to even SEQ IDs 60-304). Depending on the particular sequence, the degree of sequence identity is preferably greater than 50% (e.g. 60%, 70%, 80%, 90%, 95%, 99% or more). These proteins include homologs, orthologs, allelic variants and functional mutants. Typically, 50% identity or more between two proteins is considered to be an indication of functional equivalence. Identity between proteins is preferably determined by the Smith-Waterman homology search algorithm as implemented in the MPSRCH program (Oxford Molecular), using an affine gap search with parameters gap open penalty=12 and gap extension penalty=1.

The invention further provides proteins comprising fragments of the S. agalactiae amino acid sequences disclosed in the examples, and proteins comprising fragments of the S. pyogenes amino acid sequences disclosed in the examples (in particular to even SEQ IDs 60-304). The fragments should comprise at least n consecutive amino acids from the sequences and, depending on the particular sequence, n is 7 or more (e.g. 8, 10, 12, 14, 16, 18, 20, 30, 40, 50, 60, 70, 80, 90, 100 or more). Preferably the fragments comprise one or more epitopes from the sequence. Other preferred fragments are (a) the N-terminal signal peptides of the proteins disclosed in the examples, (b) the proteins disclosed in the examples, but without their N-terminal signal peptides, (c) fragments common to related GAS and GBS proteins disclosed in the examples, and (d) the proteins disclosed in the examples, but without their N-terminal amino acid residue.

Where the protein of the invention is related to one of SEQ IDs 2-58 or 308-346, it preferably includes one or more of the N-terminal amino acid residues which are not in the corresponding GBS (SEQ IDs 2-58) or GAS (SEQ IDs 308-346) sequences disclosed in WO02/34771 (SEQ IDs 2-58) or by Ferretti et (SEQ IDs 308-346).

Where the protein of the invention is related to SEQ ID 302, it preferably does not include residues 234-377 of G1:15674471.

Where the protein of the invention is related to SEQ ID 306, it preferably does not include residues 1-138 of G1:13621680.

The proteins of the invention can, of course, be prepared by various means (e.g. recombinant expression, purification from GAS or GBS, chemical synthesis etc.) and in various forms (e.g. native, fusions, glycosylated, non-glycosylated etc.). They are preferably prepared in substantially pure form (i.e. substantially free from other streptococcal or host cell proteins) or substantially isolated form. Proteins of the invention are preferably streptococcal proteins. They are preferred for use as vaccine antigens.

According to a further aspect, the invention provides antibodies which bind to these proteins. These may be polyclonal or monoclonal and may be produced by any suitable means (e.g. by recombinant expression). To increase compatibility with the human immune system, the antibodies may be chimeric or humanised (e.g. Breedveld (2000) Lancet 355(9205):735-740; Gorman & Clark (1990) Semin. Immunol. 2:457-466), or fully human antibodies may be used. The antibodies may include a detectable label (e.g. for diagnostic assays).

According to a further aspect, the invention provides nucleic acid comprising the S. agalactiae nucleotide sequences disclosed in the examples, and nucleic acid comprising the S. pyogenes nucleotide sequences disclosed in the examples. These nucleic acid sequences are the odd-numbered SEQ IDs between 1 and 1371.

In addition, the invention provides nucleic acid comprising nucleotide sequences having sequence identity to the S. agalactiae nucleotide sequences disclosed in the examples, and nucleic acid comprising nucleotide sequences having sequence identity to the S. pyogenes nucleotide sequences disclosed in the examples (in particular to odd SEQ IDs 59-234). Identity between sequences is preferably determined by the Smith-Waterman homology search algorithm as described above.

Furthermore, the invention provides nucleic acid which can hybridise to the S. agalactiae nucleic acid disclosed in the examples (in particular to odd SEQ IDs 59-234), and nucleic acid which can hybridise to the S. pyogenes nucleic acid disclosed in the examples preferably under ‘high stringency’ conditions (e.g. 65° C. in 0.1×SSC, 0.5% SDS solution).

Nucleic acid comprising fragments of these sequences (in particular of odd SEQ IDs 59-234) are also provided. These should comprise at least n consecutive nucleotides from the S. agalactiae or S. pyogenes sequences and, depending on the particular sequence, n is 10 or more (e.g. 12, 14, 15, 18, 20, 25, 30, 35, 40, 50, 60, 70, 80, 90, 100, 150, 200 or more). The fragments may comprise sequences which are common to related GAS and GBS sequences disclosed in the examples.

According to a further aspect, the invention provides nucleic acid encoding the proteins and protein fragments of the invention.

The invention also provides: nucleic acid comprising nucleotide sequence SEQ ID 1373; nucleic acid comprising nucleotide sequences having sequence identity to SEQ ID 1373; nucleic acid which can hybridise to SEQ ID 1373 (preferably under ‘high stringency’ conditions); nucleic acid comprising a fragment of at least n consecutive nucleotides from SEQ ID 1373, wherein n is 10 or more e.g. 12, 14, 15, 18, 20, 25, 30, 35, 40, 50, 60, 70, 80, 90, 100, 150, 200, 250, 300, 350, 400, 450, 500, 600, 700, 800, 900, 1000, 1500, 2000, 3000, 4000, 5000, 10000, 100000, 1000000 or more.

Where the nucleic acid of the invention is related to one of SEQ IDs 1-57 or 307-345, it preferably includes one or more of the 5′ nucleotide residues which are not disclosed in WO02/34771 (SEQ IDs 1-57) or by Ferretti et al. (SEQ IDs 307-345).

Where the protein of the invention is related to SEQ ID 301, it preferably does not include the nucleotides which encode amino acid residues 234-377 of G1:15674471.

Where the protein of the invention is related to SEQ ID 305, it preferably does not include the nucleotides which encode amino acid residues 1-138 of G1:13621680.

Nucleic acids of the invention can be used in hybridisation reactions (e.g. Northern or Southern blots, or in nucleic acid microarrays or ‘gene chips’) and amplification reactions (e.g. PCR, SDA, SSSR, LCR, TMA, NASBA etc.) and other nucleic acid techniques.

It should also be appreciated that the invention provides nucleic acid comprising sequences complementary to those described above (e.g. for antisense or probing, or for use as primers).

Nucleic acid according to the invention can, of course, be prepared in many ways (e.g. by chemical synthesis, from genomic or cDNA libraries, from the organism itself etc.) and can take various forms (e.g. single stranded, double stranded, vectors, primers, probes, labelled etc.). The nucleic acid is preferably in substantially isolated form.

Nucleic acid according to the invention may be labelled e.g. with a radioactive or fluorescent label. This is particularly useful where the nucleic acid is to be used in nucleic acid detection techniques e.g. where the nucleic acid is a primer or as a probe for use in techniques such as PCR, LCR, TMA, NASBA, etc.

In addition, the term “nucleic acid” includes DNA and RNA, and also their analogues, such as those containing modified backbones, and also peptide nucleic acids (PNA), etc.

According to a further aspect, the invention provides vectors comprising nucleotide sequences of the invention (e.g. cloning or expression vectors) and host cells transformed with such vectors.

According to a further aspect, the invention provides compositions comprising protein, antibody, and/or nucleic acid according to the invention. These compositions may be suitable as immunogenic compositions, for instance, or as diagnostic reagents, or as vaccines.

The invention also provides nucleic acid, protein, or antibody according to the invention for use as medicaments (e.g. as immunogenic compositions or as vaccines) or as diagnostic reagents. It also provides the use of nucleic acid, protein, or antibody according to the invention in the manufacture of: (i) a medicament for treating or preventing disease and/or infection caused by streptococcus; (ii) a diagnostic reagent for detecting the presence of streptococcus or of antibodies raised against streptococcus; and/or (iii) a reagent which can raise antibodies against streptococcus. Said streptococcus may be any species, group or strain, but is preferably S. agalactiae, especially serotype III or V, or S. pyogenes. Said disease may be bacteremia, meningitis, puerperal fever, scarlet fever, erysipelas, pharyngitis, impetigo, necrotising fasciitis, myositis or toxic shock syndrome.

The invention also provides a method of treating a patient, comprising administering to the patient a therapeutically effective amount of nucleic acid, protein, and/or antibody of the invention. The patient may either be at risk from the disease themselves or may be a pregnant woman (‘maternal immunisation’ e.g. Glezen & Alpers (1999) Clin. Infect. Dis. 28:219-224).

Administration of protein antigens is a preferred method of treatment for inducing immunity.

Administration of antibodies of the invention is another preferred method of treatment. This method of passive immunisation is particularly useful for newborn children or for pregnant women. This method will typically use monoclonal antibodies, which will be humanised or fully human.

The invention also provides a kit comprising primers (e.g. PCR primers) for amplifying a template sequence contained within a Streptococcus (e.g. S. pyogenes or S. agalactiae) nucleic acid sequence, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label (e.g. a fluorescent label).

The invention also provides a kit comprising first and second single-stranded oligonucleotides which allow amplification of a Streptococcus template nucleic acid sequence contained in a single- or double-stranded nucleic acid (or mixture thereof), wherein: (a) the first oligonucleotide comprises a primer sequence which is substantially complementary to said template nucleic acid sequence; (b) the second oligonucleotide comprises a primer sequence which is substantially complementary to the complement of said template nucleic acid sequence; (c) the first oligonucleotide and/or the second oligonucleotide comprise(s) sequence which is not compementary to said template nucleic acid; and (d) said primer sequences define the termini of the template sequence to be amplified. The non-complementary sequence(s) of feature (c) are preferably upstream of (i.e. 5′ to) the primer sequences. One or both of these (c) sequences may comprise a restriction site (e.g. EP-B-0509612) or a promoter sequence (e.g. EP-B-0505012). The first oligonucleotide and/or the second oligonucleotide may include a detectable label (e.g. a fluorescent label).

The template sequence may be any part of a genome sequence (e.g. SEQ ID 1373). For example, it could be a rRNA gene (e.g. Turenne et al. (2000) J. Clin. Microbiol. 38:513-520) or a protein-coding gene. The template sequence is preferably specific to GBS.

The invention also provides a computer-readable medium (e.g. a floppy disk, a hard disk, a CD-ROM, a DVD etc.) and/or a computer database containing one or more of the sequences in the sequence listing. The medium preferably contains SEQ ID 1373.

The invention also provides a hybrid protein represented by the formula NH₂-A-[-X-L-]ₙ-B—COOH, wherein X is a protein of the invention, L is an optional linker amino acid sequence, A is an optional N-terminal amino acid sequence, B is an optional C-terminal amino acid sequence, and n is an integer greater than 1. The value of n is between 2 and x, and the value of x is typically 3, 4, 5, 6, 7, 8, 9 or 10. Preferably n is 2, 3 or 4; it is more preferably 2 or 3; most preferably, n=2. For each n instances, —X— may be the same or different. For each n instances of [—X-L-], linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s)-L- will typically be short (e.g. 20 or fewer amino acids i.e. 19, 18, 17, 16, 15, 14, 13, 12, 11, 10, 9, 8, 7, 6, 5, 4, 3, 2, 1). Examples include short peptide sequences which facilitate cloning, poly-glycine linkers (i.e. Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art.-A- and -B- are optional sequences which will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal and C-terminal amino acid sequences will be apparent to those skilled in the art. In some embodiments, each X will be a GBS sequence; in others, mixtures of GAS and GBS will be used.

According to further aspects, the invention provides various processes.

A process for producing proteins of the invention is provided, comprising the step of culturing a host cell of to the invention under conditions which induce protein expression.

A process for producing protein or nucleic acid of the invention is provided, wherein the protein or nucleic acid is synthesised in part or in whole using chemical means.

A process for detecting polynucleotides of the invention is provided, comprising the steps of: (a) contacting a nucleic probe according to the invention with a biological sample under hybridising conditions to form duplexes; and (b) detecting said duplexes.

A process for detecting Streptococcus in a biological sample (e.g. blood) is also provided, comprising the step of contacting nucleic acid according to the invention with the biological sample under hybridising conditions. The process may involve nucleic acid amplification (e.g. PCR, SDA, SSSR, LCR, TMA, NASBA etc.) or hybridisation (e.g. microarrays, blots, hybridisation with a probe in solution etc.). PCR detection of Streptococcus in clinical samples, in particular S. pyogenes, has been reported [see e.g. Louie et al. (2000) CMAJ 163:301-309; Louie et al. (1998) J. Clin. Microbiol. 36:1769-1771]. Clinical assays based on nucleic acid are described in general in Tang et al. (1997) Clin. Chem. 43:2021-2038.

A process for detecting proteins of the invention is provided, comprising the steps of: (a) contacting an antibody of the invention with a biological sample under conditions suitable for the formation of an antibody-antigen complexes; and (b) detecting said complexes.

A process for identifying an amino acid sequence is provided, comprising the step of searching for putative open reading frames or protein-coding regions within a genome sequence of S. agalactiae. This will typically involve in silico searching the sequence for an initiation codon and for an in-frame termination codon in the downstream sequence. The region between these initiation and termination codons is a putative protein-coding sequence. Typically, all six possible reading frames will be searched. Suitable software for such analysis includes ORFFINDER (NCBI), GENEMARK [Borodovsky & McIninch (1993) Computers Chem. 17:122-133), GLIMMER [Salzberg et al. (1998) Nucleic Acids Res. 26:544-548; Salzberg et al. (1999) Genomics 59:24-31; Delcher et al. (1999) Nucleic Acids Res. 27:4636-4641], or other software which uses Markov models [e.g. Shmatkov et al. (1999) Bioinformatics 15:874-876]. The invention also provides a protein comprising the identified amino acid sequence. These proteins can then expressed using conventional techniques.

The invention also provides a process for determining whether a test compound binds to a protein of the invention. If a test compound binds to a protein of the invention and this binding inhibits the life cycle of the GBS bacterium, then the test compound can be used as an antibiotic or as a lead compound for the design of antibiotics. The process will typically comprise the steps of contacting a test compound with a protein of the invention, and determining whether the test compound binds to said protein. Preferred proteins of the invention for use in these processes are enzymes (e.g. tRNA synthetases), membrane transporters and ribosomal proteins. Suitable test compounds include proteins, polypeptides, carbohydrates, lipids, nucleic acids (e.g. DNA, RNA, and modified forms thereof), as well as small organic compounds (e.g. MW between 200 and 2000 Da). The test compounds may be provided individually, but will typically be part of a library (e.g. a combinatorial library). Methods for detecting a binding interaction include NMR, filter-binding assays, gel-retardation assays, displacement assays, surface plasmon resonance, reverse two-hybrid etc. A compound which binds to a protein of the invention can be tested for antibiotic activity by contacting the compound with GBS bacteria and then monitoring for inhibition of growth. The invention also provides a compound identified using these methods.

The invention also provides a composition comprising a protein or the invention and one or more of the following antigens:
a protein antigen from Helicobacter pylori such as VacA, CagA, NAP, HopX, HopY [e.g. WO98/04702] and/or urease.
a protein antigen from N. meningitidis serogroup B, such as those in WO99/24578, WO99/36544, WO99/57280, WO00/22430, Tettelin et al. (2000) Science 287:1809-1815, Pizza et al. (2000) Science 287:1816-1820 and WO96/29412, with protein ‘287’ and derivatives being particularly preferred.
an outer-membrane vesicle (OMV) preparation from N. meningitidis serogroup B, such as those disclosed in WO01/52885; Bjune et al. (1991) Lancet 338(8775):1093-1096; Fukasawa et al. (1999) Vaccine 17:2951-2958; Rosenqvist et al. (1998) Dev. Biol. Stand. 92:323-333 etc.
a saccharide antigen from N. meningitidis serogroup A, C, W135 and/or Y, such as the oligosaccharide disclosed in Costantino et al. (1992) Vaccine 10:691-698 from serogroup C [see also Costantino et al. (1999) Vaccine 17:1251-1263].
a saccharide antigen from Streptococcus pneumoniae [e.g. Watson (2000) Pediatr Infect Dis J 19:331-332; Rubin (2000) Pediatr Clin North Am 47:269-285, v; Jedrzejas (2001) Microbiol Mol Biol Rev 65:187-207].
an antigen from hepatitis A virus, such as inactivated virus [e.g. Bell (2000) Pediatr Infect Dis J 19:1187-1188; Iwarson (1995) APMIS 103:321-326].
an antigen from hepatitis B virus, such as the surface and/or core antigens [e.g. Gerlich et al. (1990) Vaccine 8 Suppl:S63-68 & 79-80].
an antigen from hepatitis C virus [e.g. Hsu et al. (1999) Clin Liver Dis 3:901-915].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. Gustafsson et al. (1996) N Engl. J. Med. 334:349-355; Rappuoli et al. (1991) TIBTECH 9:232-238].
a diphtheria antigen, such as a diphtheria toxoid [e.g. chapter 3 of Vaccines (1988) eds. Plotkin & Mortimer. ISBN 0-7216-1946-0] e.g. the CRM₁₉₇ mutant [e.g. Del Guidice et al. (1998) Molecular Aspects of Medicine 19:1-70].
a tetanus antigen, such as a tetanus toxoid [e.g. chapter 4 of Plotkin & Mortimer].
a saccharide antigen from Haemophilus influenzae B.
an antigen from N. gonorrhoeae [e.g. WO99/24578, WO99/36544, WO99/57280].
an antigen from Chlamydia pneumoniae [e.g. WO02/02606; Kalman et al. (1999) Nature Genetics 21:385-389; Read et al. (2000) Nucleic Acids Res 28:1397-406; Shirai et al. (2000) J. Infect. Dis. 181(Suppl 3):S524-S527; WO99/27105; WO00/27994; WO00/37494].
an antigen from Chlamydia trachomatis [e.g. WO99/28475].
an antigen from Porphyromonas gingivalis [e.g. Ross et al. (2001) Vaccine 19:4135-4142].
polio antigen(s) [e.g. Sutter et al. (2000) Pediatr Clin North Am 47:287-308; Zimmerman & Spann (1999) Am Fam Physician 59:113-118, 125-126] such as IPV or OPV.
rabies antigen(s) [e.g. Dreesen (1997) Vaccine 15 Suppl:S2-6] such as lyophilised inactivated virus [e.g. MMWR Morb Mortal Wkly Rep 1998 Jan. 16; 47(1):12, 19; RabAvert™].
measles, mumps and/or rubella antigens [e.g. chapters 9, 10 & 11 of Plotkin & Mortimer].
influenza antigen(s) [e.g. chapter 19 of Plotkin & Mortimer], such as the haemagglutinin and/or neuraminidase surface proteins.
an antigen from Moraxella catarrhalis [e.g. McMichael (2000) Vaccine 19 Suppl 1:S101-107].
an antigen from Staphylococcus aureus [e.g. Kuroda et al. (2001) Lancet 357(9264):1225-1240; see also pages 1218-1219].
an antigen disclosed in international patent application WO02/34771.
antigen(s) from a paramyxovirus such as respiratory syncytial virus (RSV e.g. Anderson (2000) Vaccine 19 Suppl 1:S59-65; Kahn (2000) Curr Opin Pediatr 12:257-262) and/or parainfluenza virus (PIV3 e.g. Crowe (1995) Vaccine 13:415-421.).
an antigen from Bacillus anthracis [e.g. Demicheli et al. (1998) Vaccine 16:880-884; Stepanov et al. (1996) J Biotechnol 44:155-160.; J Toxicol Clin Toxicol (2001) 39:85-100].
an antigen from a virus in the flaviviridae family (genus flavivirus), such as from yellow fever virus, Japanese encephalitis virus, four serotypes of Dengue viruses, tick-borne encephalitis virus, West Nile virus.
a pestivirus antigen, such as from classical porcine fever virus, bovine viral diarrhoea virus, and/or border disease virus.
a parvovirus antigen e.g. from parvovirus B19.

Where a saccharide or carbohydrate antigen is included, it is preferably conjugated to a carrier protein in order to enhance immunogenicity [e.g. Ramsay et al. (2001) Lancet 357(9251):195-196; Lindberg (1999) Vaccine 17 Suppl 2:S28-36; Conjugate Vaccines (eds. Cruse et al.) ISBN 3805549326, particularly vol. 10:48-114 etc.]. Preferred carrier proteins are bacterial toxins or toxoids, such as diphtheria or tetanus toxoids. The CRM₁₉₇ diphtheria toxoid is particularly preferred. Other suitable carrier proteins include the N. meningitidis outer membrane protein [e.g. EP-0372501], synthetic peptides [e.g. EP-0378881, EP-0427347], heat shock proteins [e.g. WO93/17712], pertussis proteins [e.g. WO98/58668; EP-0471177], protein D from H. influenzae [e.g. WO00/56360], toxin A or B from C. difficile [e.g. WO00/61761], etc. Any suitable conjugation reaction can be used, with any suitable linker where necessary.

Toxic protein antigens may be detoxified where necessary (e.g. detoxification of pertussis toxin by chemical and/or genetic means).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens are preferably adsorbed to an aluminium salt.

Antigens in the composition will typically be present at a concentration of at least 1 μg/ml each. In general, the concentration of any given antigen will be sufficient to elicit an immune response against that antigen.

The invention also provides compositions comprising two or more proteins of the present invention. The two or more proteins may comprise GBS sequences or may comprise GAS and GBS sequences. The composition can include fewer than 15 proteins of the invention (e.g. 14, 13, 12, 11, 10, 9, 8, 7, 6, 5, 4).

It is preferred that the disclosure of WO02/34771 (SEQ IDs 1 to 10967) is excluded from the scope of the present invention.

A summary of standard techniques and procedures which may be employed to perform the invention (e.g. to utilise the disclosed sequences for vaccination or diagnostic purposes) follows. This summary is not a limitation on the invention but, rather, gives examples that may be used, but are not required.

### General

The practice of the present invention will employ, unless otherwise indicated, conventional techniques of molecular biology, microbiology, recombinant DNA, and immunology, which are within the skill of the art. Such techniques are explained fully in the literature eg. Sambrook Molecular Cloning; A Laboratory Manual, Second Edition (1989); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986).

Standard abbreviations for nucleotides and amino acids are used in this specification.

### DEFINITIONS

A composition containing X is “substantially free of” Y when at least 85% by weight of the total X+Y in the composition is X. Preferably, X comprises at least about 90% by weight of the total of X+Y in the composition, more preferably at least about 95% or even 99% by weight.

The term “comprising” means “including” as well as “consisting” e.g. a composition “comprising” X may consist exclusively of X or may include something additional e.g. X+Y.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a streptococcus sequence is heterologous to a mouse host cell. A further examples would be two epitopes from the same or different proteins which have been assembled in a single protein in an arrangement not found in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

A “mutant” sequence is defined as DNA, RNA or amino acid sequence differing from but having sequence identity with the native or disclosed sequence. Depending on the particular sequence, the degree of sequence identity between the native or disclosed sequence and the mutant sequence is preferably greater than 50% (eg. 60%, 70%, 80%, 90%, 95%, 99% or more, calculated using the Smith-Waterman algorithm as described above). As used herein, an “allelic variant” of a nucleic acid molecule, or region, for which nucleic acid sequence is provided herein is a nucleic acid molecule, or region, that occurs essentially at the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions (eg. see U.S. Pat. No. 5,753,235).

### Expression Systems

The streptococcus nucleotide sequences can be expressed in a variety of different expression systems; for example those used with mammalian cells, baculoviruses, plants, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (eg. structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation [Sambrook et al. (1989) “Expression of Cloned Genes in Mammalian Cells.” In Molecular Cloning: A Laboratory Manual, 2nd ed.].

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallotheionein gene, also provide useful promoter sequences. Expression may be either constitutive or regulated (inducible), depending on the promoter can be induced with glucocorticoid in hormone-responsive cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter [Maniatis et al. (1987) Science 236:1237; Alberts et al. (1989) Molecular Biology of the Cell, 2nd ed.]. Enhancer elements derived from viruses may be particularly useful, because they usually have a broader host range. Examples include the SV40 early gene enhancer [Dijkema et al (1985) EMBO J. 4:761] and the enhancer/promoters derived from the long terminal repeat (LTR) of the Rous Sarcoma Virus [Gorman et al. (1982b) Proc. Natl. Acad. Sci. 79:6777] and from human cytomegalovirus [Boshart et al. (1985) Cell 41:521]. Additionally, some enhancers are regulatable and become active only in the presence of an inducer, such as a hormone or metal ion [Sassone-Corsi and Borelli (1986) Trends Genet. 2:215; Maniatis et al. (1987) Science 236:1237].

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus triparite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation [Bimstiel et al. (1985) Cell 41:349; Proudfoot and Whitelaw (1988) “Termination and 3′ end processing of eukaryotic RNA. In Transcription and splicing (ed. B. D. Hames and D. M. Glover); Proudfoot (1989) Trends Biochem. Sci. 14:105]. These sequences direct the transcription of an mRNA which can be translated into the polypeptide encoded by the DNA. Examples of transcription terminater/polyadenylation signals include those derived from SV40 [Sambrook et al (1989) “Expression of cloned genes in cultured mammalian cells.” In Molecular Cloning: A Laboratory Manual].

Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (eg. plasmids) capable of stable maintenance in a host, such as mammalian cells or bacteria. Mammalian replication systems include those derived from animal viruses, which require trans-acting factors to replicate. For example, plasmids containing the replication systems of papovaviruses, such as SV40 [Gluzman (1981) Cell 23:175] or polyomavirus, replicate to extremely high copy number in the presence of the appropriate viral T antigen. Additional examples of mammalian replicons include those derived from bovine papillomavirus and Epstein-Barr virus. Additionally, the replicon may have two replicaton systems, thus allowing it to be maintained, for example, in mammalian cells for expression and in a prokaryotic host for cloning and amplification. Examples of such mammalian-bacteria shuttle vectors include pMT2 [Kaufman et al. (1989) Mol. Cell. Biol. 9:946] and pHEBO [Shimizu et al. (1986) Mol. Cell. Biol. 6:1074].

The transformation procedure used depends upon the host to be transformed. Methods for introduction of heterologous polynucleotides into mammalian cells are known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (eg. Hep G2), and a number of other cell lines.

ii. Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media.

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego Calif. (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence, and transcription termination sequence, are usually assembled into an intermediate transplacement construct (transfer vector). This may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extra-chromosomal element (e.g. plasmids) capable of stable maintenance in a host, such as a bacterium. The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT; see Luckow and Summers, Virology (1989) 17:31.

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (eg. structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Virol 69:765.

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human α-interferon, Maeda et al., (1985), Nature 315:592; human gastrin-releasing peptide, Lebacq-Verheyden et al., (1988), Molec. Cell Biol. 8:3129; human IL-2, Smith et al., (1985) Proc. Nat'l Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art. (See Summers and Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91. The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between about 1% and about 5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wild-type virus, the transfection supernatant is plagued onto a monolayer of insect cells by techniques known to those skilled in the art. Namely, the plaques are screened under the light microscope for the presence (indicative of wild-type virus) or absence (indicative of recombinant virus) of occlusion bodies. “Current Protocols in Microbiology” Vol. 2 (Ausubel et al. eds) at 16.8 (Supp. 10, 1990); Summers and Smith, supra; Miller et al. (1989).

Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In vitro Cell. Dev. Biol. 25:225).

Cells and cell culture media are commercially available for both direct and fusion expression of heterologous polypeptides in a baculovirus/expression system; cell culture technology is generally known to those skilled in the art. See, eg. Summers and Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, eg. HPLC, affinity chromatography, ion exchange chromatography, etc.; electrophoresis; density gradient centrifugation; solvent extraction, etc. As appropriate, the product may be further purified, as required, so as to remove substantially any insect proteins which are also present in the medium, so as to provide a product which is at least substantially free of host debris, eg. proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iii. Plant Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture has been described by Zenk, Phytochemistry 30:3861-3863 (1991). Descriptions of plant protein signal peptides may be found in addition to the references described above in Vaulcombe et al., Mol. Gen. Genet. 209:3340 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMillin, Gibberellins: in: Advanced Plant Physiology, Malcolm B. Wilkins, ed., 1984 Pitman Publishing Limited, London, pp. 21-52. References that describe other metabolically-regulated genes: Sheen, Plant Cell, 2:1027-1038 (1990); Maas et al., EMBO 1 9:3447-3452 (1990); Henkel and Hickey, Proc. Natl. Acad. Sci. 84:1337-1339 (1987).

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11(2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl. Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iv. Bacterial Systems

Bacterial expression techniques are known in the art. A bacterial promoter is any DNA sequence capable of binding bacterial RNA polymerase and initiating the downstream (3′) transcription of a coding sequence (eg. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) [Raibaud et al. (1984) Annu. Rev. Genet. 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al. (1977) Nature 198:1056], and maltose. Additional examples include promoter sequences derived from biosynthetic enzymes such as tryptophan (tip) [Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775]. The g-laotamase (bla) promoter system [Weissmann (1981) “The cloning of interferon and other mistakes.” In Interferon 3 (ed. I. Gresser)], bacteriophage lambda PL [Shimatake et al. (1981) Nature 292:128] and TS [U.S. Pat. No. 4,689,406] promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the tac promoter is a hybrid trp-lac promoter comprised of both trp promoter and lac operon sequences that is regulated by the lac repressor [Amann et al. (1983) Gene 25:167; de Boer et al. (1983) Proc. Natl. Acad. Sci. 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al. (1986) J. Mol. Biol. 189:113; Tabor et al. (1985) Proc Natl. Acad. Sci. 82:1074]. In addition, a hybrid promoter can also be comprised of a bacteriophage promoter and an E. coli operator region (EPO-A-0 267 851).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon [Shine et al. (1975) Nature 254:34]. The SD sequence is thought to promote binding of mRNA to the ribosome by the pairing of bases between the SD sequence and the 3′ and of E. coli 16S rRNA [Steitz et al. (1979) “Genetic signals and nucleotide sequences in messenger RNA.” In Biological Regulation and Development: Gene Expression (ed. R. F. Goldberger)]. To express eukaryotic genes and prokaryotic genes with weak ribosome-binding site [Sambrook et al. (1989) “Expression of cloned genes in Escherichia coli.” In Molecular Cloning: A Laboratory Manual].

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EP-A-0 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene [Nagai et al. (1984) Nature 309:810]. Fusion proteins can also be made with sequences from the lacZ [Jia et al. (1987) Gene 60:197], trpE [Allen et al. (1987) J. Biotechnol. 5:93; Makoff et al. (1989) J. Gen. Microbiol. 135:11], and Chey [EP-A-0 324 647] genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (eg. ubiquitin specific processing-protease) to cleave the ubiquitin from the foreign protein. Through this method, native foreign protein can be isolated [Miller et al. (1989) Bio/Technology 7:698].

Alternatively, foreign proteins can also be secreted from the cell by creating chimeric DNA molecules that encode a fusion protein comprised of a signal peptide sequence fragment that provides for secretion of the foreign protein in bacteria [U.S. Pat. No. 4,336,336]. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) [Masui et al. (1983), in: Experimental Manipulation of Gene Expression; Ghrayeb et al. (1984) EMBO J. 3:2437] and the E. coli alkaline phosphatase signal sequence (phoA) [Oka et al. (1985) Proc. Nail Acad. Sci. 82:7212]. As an additional example, the signal sequence of the alpha-amylase gene from various Bacillus strains can be used to secrete heterologous proteins from B. subtilis [Palva et al. (1982) Proc. Natl. Acad. Sci. USA 79:5582; EP-A-0 244 042].

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (eg. plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (EP-A-0 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline [Davies et al. (1978) Annu. Rev. Microbiol. 32:469]. Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis [Palva et al. (1982) Proc. Natl. Acad. Sci. USA 79:5582; EP-A-0 036 259 and EP-A-0 063 953; WO 84/04541], Escherichia coli [Shimatake et al. (1981) Nature 292:128; Amann et al. (1985) Gene 40:183; Studier et al. (1986) J. Mol. Biol. 189:113; EP-A-0 036 776, EP-A-0 136 829 and EP-A-0 136 907], Streptococcus cremoris [Powell et al. (1988) Appl. Environ. Microbiol. 54:655]; Streptococcus lividans [Powell et al. (1988) Appl. Environ. Microbiol. 54:655], Streptomyces lividans [U.S. Pat. No. 4,745,056].

Methods of introducing exogenous DNA into bacterial hosts are well-known in the art, and usually include either the transformation of bacteria treated with CaCl.sub.2 or other agents, such as divalent cations and DMSO. DNA can also be introduced into bacterial cells by electroporation. Transformation procedures usually vary with the bacterial species to be transformed. See eg. [Masson et al. (1989) FEMS Microbiol. Lett. 60:273; Palva et al. (1982) Proc. Natl. Acad. Sci. USA 79:5582; EP-A-0 036 259 and EP-A-0 063 953; WO 84/04541, Bacillus], [Miller et al. (1988) Proc. Natl. Acad. Sci. 85:856; Wang et al. (1990) J. Bacteriol. 172:949, Campylobacter], [Cohen et al. (1973) Proc. Natl. Acad. Sci. 69:2110; Dower et al. (1988) Nucleic Acids Res. 16:6127; Kushner (1978) “An improved method for transformation of Escherichia coli with ColE 1-derived plasmids. In Genetic Engineering: Proceedings of the International Symposium on Genetic Engineering (eds. H. W. Boyer and S, Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al. (1980) J. Bacteriol. 144:698; Harlander (1987) “Transformation of Streptococcus lactis by electroporation, in: Streptococcal Genetics (ed. J. Ferretti and R. Curtiss III); Perry et al., (1981) Infect. Immun. 32:1295; Powell et al. (1988) Appl. Environ. Microbiol. 54:655; Somkuti et al. (1987) Proc. 4th Evr. Cong. Biotechnology 1:412, Streptococcus].

v. Yeast Expression

Yeast expression systems are also known to one of ordinary skill in the art. A yeast promoter is any DNA sequence capable of binding yeast RNA polymerase and initiating the downstream (3′) transcription of a coding sequence (eg. structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EP-A-0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO-A-0 329 203). The yeast PHO5 gene, encoding acid phosphatase, also provides useful promoter sequences [Myanohara et al. (1983) Proc. Natl. Acad. Sci. USA 80:1].

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK (EP-A-0 164 556). Furthermore, a yeast promoter can include naturally occurring promoters of non-yeast origin that have the ability to bind yeast RNA polymerase and initiate transcription. Examples of such promoters include, inter alia, [Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109;].

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See eg. EP-A-0 196 056. Another example is a ubiquitin fusion protein. Such a fusion protein is made with the ubiquitin region that preferably retains a site for a processing enzyme (eg. ubiquitin-specific processing protease) to cleave the ubiquitin from the foreign protein. Through this method, therefore, native foreign protein can be isolated (eg. WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the yeast invertase gene (EP-A-0 012 873; JPO. 62,096,086) and the A-factor gene (U.S. Pat. No. 4,588,684). Alternatively, leaders of non-yeast origin, such as an interferon leader, exist that also provide for secretion in yeast (EP-A-0 060 057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EP-A-0 324 274). Additional leaders employing an alpha-factor leader fragment that provides for secretion include hybrid alpha-factor leaders made with a presequence of a first yeast, but a pro-region from a second yeast alphafactor. (eg. see WO 89/02463.)

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (eg. plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 [Botstein et al. (1979) Gene 8:17-24], pCl/1 [Brake et al. (1984) Proc. Natl. Acad. Sci. USA 81:4642-4646], and YRp17 [Stinchcomb et al. (1982) J. Mol. Biol. 158:157]. In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See eg. Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome [On-Weaver et al. (1983) Methods in Enzymol. 101:228-245]. An integrating vector may be directed to a specific locus in yeast by selecting the appropriate homologous sequence for inclusion in the vector. See On-Weaver et al., supra. One or more expression construct may integrate, possibly affecting levels of recombinant protein produced [Rine et al. (1983) Proc. Natl. Acad. Sci. USA 80:6750]. The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions [Butt et al. (1987) Microbiol, Rev. 51:351].

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors have been developed for, inter alia, the following yeasts: Candida albicans [Kurtz, et al. (1986) Mol. Cell. Biol. 6:142], Candida maltosa [Kunze, et al. (1985) J. Basic Microbiol. 25:141]. Hansenula polymorpha [Gleeson, et al. (1986) J. Gen. Microbiol. 132:3459; Roggenkamp et al. (1986) Mol. Gen. Genet. 202:302], Kluyveromyces fragilis [Das, et al. (1984) J. Bacteriol. 158:1165], Kluyveromyces lactis [De Louvencourt et al. (1983) J. Bacteriol. 154:737; Van den Berg et al. (1990) Bio/Technology 8:135], Pichia guillerimondii [Kunze et al. (1985) J. Basic Microbiol. 25:141], Pichia pastoris [Cregg, et al. (1985) Mol. Cell. Biol. 5:3376; U.S. Pat. Nos. 4,837,148 and 4,929,555], Saccharomyces cerevisiae [Hinnen et al. (1978) Proc. Natl. Acad. Sci. USA 75:1929; Ito et al. (1983) J. Bacteriol. 153:163], Schizosaccharomyces pombe [Beach and Nurse (1981) Nature 300:706], and Yarrowia lipolytica [Davidow, et al. (1985) Curr. Genet. 10:380471 Gaillardin, et al. (1985) Curr. Genet. 10:49].

Methods of introducing exogenous DNA into yeast hosts are well-known in the art, and usually include either the transformation of spheroplasts or of intact yeast cells treated with alkali cations. Transformation procedures usually vary with the yeast species to be transformed. See eg. [Kurtz et al. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (1984) J. Bacteriol. 158:1165; De Louvencourt et al. (1983) J. Bacteriol. 154:1165; Van den Berg et al. (1990) Bio/Technology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 and 4,929,555; Pichia]; [Hinnen et al. (1978) Proc. Natl. Acad. Sci. USA 75; 1929; Ito et al. (1983) J. Bacteriol. 153:163 Saccharomyces]; [Beach and Nurse (1981) Nature 300:706; Schizosaccharomyces]; [Davidow et al. (1985) Curr. Genet. 10:39; Gaillardin et al. (1985) Curr. Genet. 10:49; Yarrowia].

### Antibodies

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

Antibodies against the proteins of the invention are useful for affinity chromatography, immunoassays, and distinguishing/identifying streptococcus proteins.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 mu.g/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antisera is obtained by bleeding the immunized animal into a glass or plastic container, incubating the blood at 25.degree. C. for one hour, followed by incubating at 4.degree. C. for 2-18 hours. The serum is recovered by centrifugation (eg. 1,000 g for 10 minutes). About 20-50 ml per bleed may be obtained from rabbits.

Monoclonal antibodies are prepared using the standard method of Kohler & Milstein [Nature (1975) 256:495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (eg. hypoxanthine, aminopterin, thymidine medium, “HAT”). The resulting hybridomas are plated by limiting dilution, and are assayed for production of antibodies which bind specifically to the immunizing antigen (and which do not bind to unrelated antigens). The selected MAb-secreting hybridomas are then cultured either in vitro (eg. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly .sup.32P and .sup.125I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, .sup.125I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with .sup.125I, or with an anti-biotin MAb labeled with HRP. Other permutations and possibilities will be readily apparent to those of ordinary skill in the art, and are considered as equivalents within the scope of the instant invention.

### Pharmaceutical Compositions

Pharmaceutical compositions can comprise either polypeptides, antibodies, or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the molecule of the invention in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

### Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (eg. see WO98/20734), needles, and gene guns or hyposprays. Dosage treatment may be a single dose schedule or a multiple dose schedule.

### Vaccines

Vaccines according to the invention may either be prophylactic (ie. to prevent infection) or therapeutic (ie. to treat disease after infection).

Such vaccines comprise immunising antigen(s), immunogen(s), polypeptide(s), protein(s) or nucleic acid, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogens.

Preferred adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (WO90/14837; Chapter 10 in Vaccine Design—the subunit and adjuvant approach (1995) ed. Powell & Newman), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (2) saponin adjuvants, such as QS21 or Stimulon™ (Cambridge Bioscience, Worcester, Mass.) may be used or particles generated therefrom such as ISCOMs (immunostimulating complexes), which ISCOMS may be devoid of additional detergent e.g. WO00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454; (6) combinations of 3dMPL with, for example, QS21 and/or oil-in-water emulsions e.g. EP-A-0835318, EP-A-0735898, EP-A-0761231; (7) oligonucleotides comprising CpG motifs [Krieg Vaccine 2000, 19, 618-622; Krieg Curr opin Mol Ther 2001 3:15-24; Roman et al., Nat. Med., 1997, 3, 849-854; Weiner et al., PNAS USA, 1997, 94, 10833-10837; Davis et al., J. Immunol., 1998, 160, 870-876; Chu et al., J. Exp. Med, 1997, 186, 1623-1631; Lipford et al., Eur. J. Immunol., 1997, 27, 2340-2344; Moldoveanu et al., Vaccine, 1988, 16, 1216-1224, Krieg et al, Nature, 1995, 374, 546-549; Klinman et al, PNAS USA, 1996, 93, 2879-2883; Ballas et al., J. Immunol., 1996, 157, 1840-1845; Cowdery et al., J. Immunol., 1996, 156, 4570-4575; Halpern et al., Cell. Immunol., 1996, 167, 72-78; Yamamoto et al., Jpn. J. Cancer Res., 1988, 79, 866-873; Stacey et al., J. Immunol., 1996, 157, 2116-2122; Messina et al, J. Immunol., 1991, 147, 1759-1764; Yi et al, J. Immunol., 1996, 157, 4918-4925; Yi et al., J. Immunol., 1996, 157, 5394-5402; Yi et al, J. Immunol., 1998, 160, 4755-4761; and Yi et al., J. Immunol, 1998, 160, 5898-5906; International patent applications WO96/02555, WO98/16247, WO98/18810, WO98/40100, WO98/55495, WO98/37919 and WO98/52581] .e. containing at least one CG dinucleotide, with 5-methylcytosine optionally being used in place of cytosine; (8) a polyoxyethylene ether or a polyoxyethylene ester e.g. WO99/52549; (9) a polyoxyethylene sorbitan ester surfactant in combination with an octoxynol (e.g. WO01/21207) or a polyoxyethylene alkyl ether or ester surfactant in combination with at least one additional non-ionic surfactant such as an octoxynol (e.g. WO01/21152); (10) an immunostimulatory oligonucleotide (e.g. a CpG oligonucleotide) and a saponin e.g. WO00/62800; (11) an immunostimulant and a particle of metal salt e.g. WO00/23105; (12) a saponin and an oil-in-water emulsion e.g. WO99/11241; (13) a saponin (e.g. QS21)+3dMPL+IL-12 (optionally+a sterol) e.g. WO98/57659; (14) aluminium salts, preferably hydroxide or phosphate, but any other suitable salt may also be used (e.g. hydroxyphosphate, oxyhydroxide, orthophosphate, sulphate etc. [e.g. see chapters 8 & 9 of Powell & Newman]). Mixtures of different aluminium salts may also be used. The salt may take any suitable form (e.g. gel, crystalline, amorphous etc.); (15) other substances that act as immunostimulating agents to enhance the efficacy of the composition.

Further adjuvants which may be used are: (1) microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid) such as poly(lactide-co-glycolide), a polyhydroxybutyric acid, a polyorthoester, a polyanhydride, a polycaprolactone etc.). optionally treated to have a negatively-charged surface (eg. with SDS) or a positively-charged surface (e.g. with a cationic detergent, such as CTAB); (2) E. coli heat-labile enterotoxin (“LT”), or detoxified mutants thereof, such as the K63 or R72 mutants [e.g. Chapter 5 of Del Giudice et al. (1998) Molecular Aspects of Medicine, vol. 19, number 1.]; (3) cholera toxin (“CT”), or detoxified mutants thereof [e.g. Del Giudice et al., supra]; (4) liposomes; (5) double-stranded RNA; (6) monophosphoryl lipid A mimics, such as aminoalkyl glucosaminide phosphate derivatives e.g. RC-529 [Johnson et al (1999) Bioorg Med Chem Lett 9:2273-2278.]; (7) polyphosphazene (PCPP); (8) a bioadhesive [WO00/50078] such as esterified hyaluronic acid microspheres [Singh et al. (2001) J. Cont. Rele. 70:267-276.] or a mucoadhesive selected from the group consisting of cross-linked derivatives of poly(acrylic acid), polyvinyl alcohol, polyvinyl pyrollidone, polysaccharides and carboxymethylcellulose.

Aluminium salts are preferred adjuvants. Where an aluminium salt it used, it is possible to adsorb one or more of the antigens to the aluminium salt.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-241′-2′-dipalmitoyl-s-n-glycero-3-hydroxyphosphoryloxy]ethylamine (MTP-PE), etc.

The immunogenic compositions (eg. the immunising antigen/immunogen/polypeptide/protein/nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (eg. nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The immunogenic compositions are conventionally administered parenterally, eg. by injection, either subcutaneously, intramuscularly, or transdermally/transcutaneously (eg. WO98/20734). Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

As an alternative to protein-based vaccines, DNA vaccination may be used [eg. Robinson & Torres (1997) Seminars in Immunol 9:271-283; Donnelly et al. (1997) Annu Rev Immunol 15:617-648; later herein].

### Gene Delivery Vehicles

Gene therapy vehicles for delivery of constructs including a coding sequence of a therapeutic of the invention, to be delivered to the mammal for expression in the mammal, can be administered either locally or systemically. These constructs can utilize viral or non-viral vector approaches in in vivo or ex vivo modality. Expression of such coding sequence can be induced using endogenous mammalian or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-44; Kimura (1994) Human Gene Therapy 5:845-852; Connelly (1995) Human Gene Therapy 6:185-193; and Kaplitt (1994) Nature Genetics 6:148-153.

Retroviral vectors are well known in the art and we contemplate that any retroviral gene therapy vector is employable in the invention, including B, C and D type retroviruses, xenotropic retroviruses (for example, NZB-X1, NZB-X2 and NZB9-1 (see O'Neill (1985) J. Virol. 53:160) polytropic retroviruses eg. MCF and MCF-MLV (see Kelly (1983) J. Virol. 45:291), spumaviruses and lentiviruses. See RNA Tumor Viruses, Second Edition, Cold Spring Harbor Laboratory, 1985.

Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Pat. No. 5,591,624). Retrovirus vectors can be constructed for site-specific integration into host cell DNA by incorporation of a chimeric integrase enzyme into the retroviral particle (see WO96/37626). It is preferable that the recombinant viral vector is a replication defective recombinant virus.

Packaging cell lines suitable for use with the above-described retrovirus vectors are well known in the art, are readily prepared (see WO95/30763 and WO92/05266), and can be used to create producer cell lines (also termed vector cell lines or “VCLs”) for the production of recombinant vector particles. Preferably, the packaging cell lines are made from human parent cells (eg. HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J Virol 19:19-25), Abelson (ATCC No. VR-999), Friend (ATCC No. VR-245), Graffi, Gross (ATCC Nol VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Exemplary known retroviral gene therapy vectors employable in this invention include those described in patent applications GB2200651, EP0415731, EP0345242, EP0334301, WO89/02468; WO89/05349, WO89/09271, WO90/02806, WO90/07936, WO94/03622, WO93/25698, WO93/25234, WO93/11230, WO93/10218, WO91/02805, WO91/02825, WO95/07994, U.S. Pat. No. 5,219,740, U.S. Pat. No. 4,405,712, U.S. Pat. No. 4,861,719, U.S. Pat. No. 4,980,289, U.S. Pat. No. 4,777,127, U.S. Pat. No. 5,591,624. See also Vile (1993) Cancer Res 53:3860-3864; Vile (1993) Cancer Res 53:962-967; Ram (1993) Cancer Res 53 (1993) 83-88; Takamiya (1992) Neurosci Res 33:493-503; Baba (1993) J Neurosurg 79:729-735; Mann (1983) Cell 33:153; Cane (1984) Proc Natl Acad Sci 81:6349; and Miller (1990) Human Gene Therapy 1.

Human adenoviral gene therapy vectors are also known in the art and employable in this invention. See, for example, Berkner (1988) Biotechniques 6:616 and Rosenfeld (1991) Science 252:431, and WO93/07283, WO93/06223, and WO93/07282. Exemplary known adenoviral gene therapy vectors employable in this invention include those described in the above referenced documents and in WO94/12649, WO93/03769, WO93/19191, WO94/28938, WO95/11984, WO95/00655, WO95/27071, WO95/29993, WO95/34671, WO96/05320, WO94/08026, WO94/11506, WO93/06223, WO94/24299, WO95/14102, WO95/24297, WO95/02697, WO94/28152, WO94/24299, WO95/09241, WO95/25807, WO95/05835, WO94/18922 and WO95/09654. Alternatively, administration of DNA linked to killed adenovirus as described in Curiel (1992) Hum. Gene Ther. 3:147-154 may be employed. The gene delivery vehicles of the invention also include adenovirus associated virus (AAV) vectors. Leading and preferred examples of such vectors for use in this invention are the AAV-2 based vectors disclosed in Srivastava, WO93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (ie. there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini (1993) Gene 124:257-262. Another example of such an AAV vector is psub201 (see Samulski (1987) J. Virol. 61:3096). Another exemplary AAV vector is the Double-D ITR vector. Construction of the Double-D ITR vector is disclosed in U.S. Pat. No. 5,478,745. Still other vectors are those disclosed in Carter U.S. Pat. No. 4,797,368 and Muzyczka U.S. Pat. No. 5,139,941, Chartejee U.S. Pat. No. 5,474,935, and Kotin WO94/288157. Yet a further example of an AAV vector employable in this invention is SSV9AFABTKneo, which contains the AFP enhancer and albumin promoter and directs expression predominantly in the liver. Its structure and construction are disclosed in Su (1996) Human Gene Therapy 7:463-470. Additional AAV gene therapy vectors are described in U.S. Pat. No. 5,354,678, U.S. Pat. No. 5,173,414, U.S. Pat. No. 5,139,941, and U.S. Pat. No. 5,252,479.

The gene therapy vectors of the invention also include herpes vectors. Leading and preferred examples are herpes simplex virus vectors containing a sequence encoding a thymidine kinase polypeptide such as those disclosed in U.S. Pat. No. 5,288,641 and EP0176170 (Roizman). Additional exemplary herpes simplex virus vectors include HFEM/ICP6-LacZ disclosed in WO95/04139 (Wistar Institute), pHSVlac described in Geller (1988) Science 241:1667-1669 and in WO90/09441 and WO92/07945, HSV Us3::pgC-lacZ described in Fink (1992) Human Gene Therapy 3:11-19 and HSV 7134, 2 RH 105 and GAL4 described in EP 0453242 (Breakefield), and those deposited with the ATCC with accession numbers VR-977 and VR-260.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO92/10578. More particularly, those alpha virus vectors described in U.S. Ser. No. 08/405,627, filed Mar. 15, 1995, WO94/21792, WO92/10578, WO95/07994, U.S. Pat. No. 5,091,309 and U.S. Pat. No. 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see U.S. Ser. No. 08/679,640).

DNA vector systems such as eukaryotic layered expression systems are also useful for expressing the nucleic acids of the invention. See WO95/07994 for a detailed description of eukaryotic layered expression systems. Preferably, the eukaryotic layered expression systems of the invention are derived from alphavirus vectors and most preferably from Sindbis viral vectors.

Other viral vectors suitable for use in the present invention include those derived from poliovirus, for example ATCC VR-58 and those described in Evans, Nature 339 (1989) 385 and Sabin (1973) J. Biol. Standardization 1:115; rhinovirus, for example ATCC VR-1110 and those described in Arnold (1990) J. Cell Biochem L401; pox viruses such as canary pox virus or vaccinia virus, for example ATCC VR-111 and ATCC VR-2010 and those described in Fisher-Hoch (1989) Proc Natl Acad Sci 86:317; Flexner (1989) Ann NY Acad Sci 569:86, Flexner (1990) Vaccine 8:17; in U.S. Pat. No. 4,603,112 and U.S. Pat. No. 4,769,330 and WO89/01973; SV40 virus, for example ATCC VR-305 and those described in Mulligan (1979) Nature 277:108 and Madzak (1992) J Gen Virol 73:1533; influenza virus, for example ATCC VR-797 and recombinant influenza viruses made employing reverse genetics techniques as described in U.S. Pat. No. 5,166,057 and in Enami (1990) Proc Natl Acad Sci 87:3802-3805; Enami & Palese (1991) J Virol 65:2711-2713 and Luytjes (1989) Cell 59:110, (see also McMichael (1983) NEJ Med 309:13, and Yap (1978) Nature 273:238 and Nature (1979) 277:108); human immunodeficiency virus as described in EP-0386882 and in Buchschacher (1992) J. Virol. 66:2731; measles virus, for example ATCC VR-67 and VR-1247 and those described in EP-0440219; Aura virus, for example ATCC VR-368; Bebaru virus, for example ATCC VR-600 and ATCC VR-1240; Cabassou virus, for example ATCC VR-922; Chikungunya virus, for example ATCC VR-64 and ATCC VR-1241; Fort Morgan Virus, for example ATCC VR-924; Getah virus, for example ATCC VR-369 and ATCC VR-1243; Kyzylagach virus, for example ATCC VR-927; Mayaro virus, for example ATCC VR-66; Mucambo virus, for example ATCC VR-580 and ATCC VR-1244; Ndumu virus, for example ATCC VR-371; Pixuna virus, for example ATCC VR-372 and ATCC VR-1245; Tonate virus, for example ATCC VR-925; Triniti virus, for example ATCC VR-469; Una virus, for example ATCC VR-374; Whataroa virus, for example ATCC VR-926; Y-62-33 virus, for example ATCC VR-375; O'Nyong virus, Eastern encephalitis virus, for example ATCC VR-65 and ATCC VR-1242; Western encephalitis virus, for example ATCC VR-70, ATCC VR-1251, ATCC VR-622 and ATCC VR-1252; and coronavirus, for example ATCC VR-740 and those described in Hamre (1966) Proc Soc Exp Biol Med 121:190.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994 and Curiel (1992) Hum Gene Ther 3:147-154 ligand linked DNA, for example see Wu (1989) J Biol Chem 264:16985-16987, eucaryotic cell delivery vehicles cells, for example see U.S. Ser. No. 08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in WO92/11033, nucleic charge neutralization or fusion with cell membranes. Additional approaches are described in Philip (1994) Mol Cell Biol 14:2411-2418 and in Woffendin (1994) Proc Natl Acad Sci 91:1581-1585.

Particle mediated gene transfer may be employed, for example see U.S. Ser. No. 60/023,867. Briefly, the sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO95/13796, WO94/23697, WO91/14445 and EP-524,968. As described in U.S. Ser. No. 60/023,867, on non-viral delivery, the nucleic acid sequences encoding a polypeptide can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO92/11033

Exemplary liposome and polycationic gene delivery vehicles are those described in U.S. Pat. Nos. 5,422,120 and 4,762,915; in WO 95/13796; WO94/23697; and WO91/14445; in EP-0524968; and in Stryer, Biochemistry, pages 236-240 (1975) W.H. Freeman, San Francisco; Szoka (1980) Biochem Biophys Acta 600:1; Bayer (1979) Biochem Biophys Acta 550:464; Rivnay (1987) Meth Enzymol 149:119; Wang (1987) Proc Natl Acad Sci 84:7851; Plant (1989) Anal Biochem 176:420.

A polynucleotide composition can comprises therapeutically effective amount of a gene therapy vehicle, as the term is defined above. For purposes of the present invention, an effective dose will be from about 0.01 mg/kg to 50 mg/kg or 0.05 mg/kg to about 10 mg/kg of the DNA constructs in the individual to which it is administered.

### Delivery Methods

Once formulated, the polynucleotide compositions of the invention can be administered (1) directly to the subject; (2) delivered ex vivo, to cells derived from the subject; or (3) in vitro for expression of recombinant proteins. The subjects to be treated can be mammals or birds. Also, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (eg. see WO98/20734), needles, and gene guns or hyposprays. Dosage treatment may be a single dose schedule or a multiple dose schedule.

Methods for the ex vivo delivery and reimplantation of transformed cells into a subject are known in the art and described in eg. WO93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

### Polynucleotide and Polypeptide Pharmaceutical Compositions

In addition to the pharmaceutically acceptable carriers and salts described above, the following additional agents can be used with polynucleotide and/or polypeptide compositions.

### A. Polypeptides

One example are polypeptides which include, without limitation: asioloorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

### B. Hormones, Vitamins, etc.

Other groups that can be included are, for example: hormones, steroids, androgens, estrogens, thyroid hormone, or vitamins, folic acid.

### C. Polyalkylenes, Polysaccharides, etc.

Also, polyalkylene glycol can be included with the desired polynucleotides/polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide)

### D. Lipids, and Liposomes

The desired polynucleotide/polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Felgner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N[-2,3-dioleyloxy)propyl]-N,N,N-triethylammonium (DOTMA) liposomes are available under the trademark Lipofectin, from GIBCO BRL, Grand Island, N.Y. (See, also, Felgner supra). Other commercially available liposomes include transfectace (DDAB/DOPE) and DOTAP/DOPE (Boerhinger). Other cationic liposomes can be prepared from readily available materials using techniques well known in the art. See, eg. Szoka (1978) Proc. Natl. Acad. Sci. USA 75:4194-4198; WO90/11092 for a description of the synthesis of DOTAP (1,2-bis(oleoyloxy)-3-(trimethylammonio)propane)liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See eg. Straubinger (1983) Meth. Immunol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

### E. Lipoproteins

In addition, lipoproteins can be included with the polynucleotide/polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein. For example, naturally occurring chylomicrons comprises of A, B, C & E, over time these lipoproteins lose A and acquire C & E. VLDL comprises A, B, C & E apoproteins, LDL comprises apoprotein B; and HDL comprises apoproteins A, C, & E.

The amino acid of these apoproteins are known and are described in, for example, Breslow (1985) Annu Rev. Biochem 54:699; Law (1986) Adv. Exp Med. Biol. 151:162; Chen (1986) J Biol Chem 261:12918; Kane (1980) Proc Natl Acad Sci USA 77:2465; and Utermann (1984) Hum Genet. 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mahey (1979) J. Clin. Invest 64:743-750. Lipoproteins can also be produced by in vitro or recombinant methods by expression of the apoprotein genes in a desired host cell. See, for example, Atkinson (1986) Annu Rev Biophys Chem 15:403 and Radding (1958) Biochim Biophys Acta 30: 443. Lipoproteins can also be purchased from commercial suppliers, such as Biomedical Techniologies, Inc., Stoughton, Mass., USA. Further description of lipoproteins can be found in WO98/06437.

### F. Polycationic Agents

Polycationic agents can be included, with or without lipoprotein, in a composition with the desired polynucleotide/polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples include histones, protamines, human serum albumin, DNA binding proteins, non-histone chromosomal proteins, coat proteins from DNA viruses, such as (X174, transcriptional factors also contain domains that bind DNA and therefore may be useful as nucleic aid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spermine, spermidine, and purtrescine.

The dimensions and of the physical properties of a polycationic agent can be extrapolated from the list above, to construct other polypeptide polycationic agents or to produce synthetic polycationic agents.

Synthetic polycationic agents which are useful include, for example, DEAE-dextran, polybrene. Lipofectin™, and lipofectAMINE™ are monomers that form polycationic complexes when combined with polynucleotides/polypeptides.

### Immunodiagnostic Assays

Streptococcus antigens of the invention can be used in immunoassays to detect antibody levels (or, conversely, anti-streptococcus antibodies can be used to detect antigen levels). Immunoassays based on well defined, recombinant antigens can be developed to replace invasive diagnostics methods. Antibodies to streptococcus proteins within biological samples, including for example, blood or serum samples, can be detected. Design of the immunoassays is subject to a great deal of variation, and a variety of these are known in the art. Protocols for the immunoassay may be based, for example, upon competition, or direct reaction, or sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Kits suitable for immunodiagnosis and containing the appropriate labeled reagents are constructed by packaging the appropriate materials, including the compositions of the invention, in suitable containers, along with the remaining reagents and materials (for example, suitable buffers, salt solutions, etc.) required for the conduct of the assay, as well as suitable set of assay instructions.

### Nucleic Acid Hybridisation

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al. [supra] Volume 2, chapter 9, pages 9.47 to 9.57.

“Stringency” refers to conditions in a hybridization reaction that favor association of very similar sequences over sequences that differ. For example, the combination of temperature and salt concentration should be chosen that is approximately 120 to 200.degree. C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook et al. at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 μg for a plasmid or phage digest to 10.sup.-9 to 10.sup.-8 g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1.mu.g of yeast DNA, blotting for two hours, and hybridizing for 4-8 hours with a probe of 10.sup.8 cpm/.mu.g. For a single-copy mammalian gene a conservative approach would start with 10.mu.g of DNA, blot overnight, and hybridize overnight in the presence of 10% dextran sulfate using a probe of greater than 10.sup.8 cpm/.mu.g, resulting in an exposure time of ˜24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:

Tₘ=81+16.6(log₁₀Ci)+0.4[% (G+C)]−0.6(% formamide)−600/n−1.5(% mismatch).

where Ci is the salt concentration (monovalent ions) and n is the length of the hybrid in base pairs (slightly modified from Meinkoth & Wahl (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (ie. stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and reexposed. If the time required for exposure makes this approach impractical, several hybridization and/or washing stringencies should be tested in parallel.

### Nucleic Acid Probe Assays

Methods such as PCR, branched DNA probe assays, or blotting techniques utilizing nucleic acid probes according to the invention can determine the presence of cDNA or mRNA. A probe is said to “hybridize” with a sequence of the invention if it can form a duplex or double stranded complex, which is stable enough to be detected.

The nucleic acid probes will hybridize to the streptococcus nucleotide sequences of the invention (including both sense and antisense strands). Though many different nucleotide sequences will encode the amino acid sequence, the native streptococcus sequence is preferred because it is the actual sequence present in cells. mRNA represents a coding sequence and so a probe should be complementary to the coding sequence; single-stranded cDNA is complementary to mRNA, and so a cDNA probe should be complementary to the non-coding sequence.

The probe sequence need not be identical to the streptococcus sequence (or its complement)—some variation in the sequence and length can lead to increased assay sensitivity if the nucleic acid probe can form a duplex with target nucleotides, which can be detected. Also, the nucleic acid probe can include additional nucleotides to stabilize the formed duplex. Additional streptococcus sequence may also be helpful as a label to detect the formed duplex. For example, a non-complementary nucleotide sequence may be attached to the 5′ end of the probe, with the remainder of the probe sequence being complementary to a streptococcus sequence. Alternatively, non-complementary bases or longer sequences can be interspersed into the probe, provided that the probe sequence has sufficient complementarity with the a streptococcus sequence in order to hybridize therewith and thereby form a duplex which can be detected.

The exact length and sequence of the probe will depend on the hybridization conditions (e.g. temperature, salt condition etc.). For example, for diagnostic applications, depending on the complexity of the analyte sequence, the nucleic acid probe typically contains at least 10-20 nucleotides, preferably 15-25, and more preferably at least 30 nucleotides, although it may be shorter than this. Short primers generally require cooler temperatures to form sufficiently stable hybrid complexes with the template.

Probes may be produced by synthetic procedures, such as the triester method of Matteucci et al. [J. Am. Chem. Soc. (1981) 103:3185], or according to Urdea et al. [Proc. Natl. Acad. Sci. USA (1983) 80: 7461], or using commercially available automated oligonucleotide synthesizers.

The chemical nature of the probe can be selected according to preference. For certain applications, DNA or RNA are appropriate. For other applications, modifications may be incorporated eg. backbone modifications, such as phosphorothioates or methylphosphonates, can be used to increase in vivo half-life, alter RNA affinity, increase nuclease resistance etc. [eg. see Agrawal & Iyer (1995) Curr Opin Biotechnol 6:12-19; Agrawal (1996) TIBTECH 14:376-387]; analogues such as peptide nucleic acids may also be used [eg. see Corey (1997) TIBTECH 15:224-229; Buchardt et al. (1993) TIBTECH 11:384-386].

Alternatively, the polymerase chain reaction (PCR) is another well-known means for detecting small amounts of target nucleic acid. The assay is described in Mullis et al. [Meth. Enzymol. (1987) 155:335-350] & U.S. Pat. Nos. 4,683,195 & 4,683,202. Two “primer” nucleotides hybridize with the target nucleic acids and are used to prime the reaction. The primers can comprise sequence that does not hybridize to the sequence of the amplification target (or its complement) to aid with duplex stability or, for example, to incorporate a convenient restriction site. Typically, such sequence will flank the desired streptococcus sequence.

A thermostable polymerase creates copies of target nucleic acids from the primers using the original target nucleic acids as a template. After a threshold amount of target nucleic acids are generated by the polymerase, they can bedetected by more traditional methods, such as Southern blots. When using the Southern blot method, the labelled probe will hybridize to the streptococcus sequence (or its complement).

Also, mRNA or cDNA can be detected by traditional blotting techniques described in Sambrook et al [supra]. mRNA, or cDNA generated from mRNA using a polymerase enzyme, can be purified and separated using gel electrophoresis. The nucleic acids on the gel are then blotted onto a solid support, such as nitrocellulose. The solid support is exposed to a labelled probe and then washed to remove any unhybridized probe. Next, the duplexes containing the labeled probe are detected. Typically, the probe is labelled with a radioactive moiety.

### BRIEF DESCRIPTION OF DRAWINGS

There are no drawings.

### MODES FOR CARRYING OUT THE INVENTION

The following examples describe nucleic acid and/or amino acid sequences which have been identified in GAS or GBS, along with their inferred translation products.

### Details of the Examples are as Follows:

Examples 1 to 29 describe variants of GBS sequences from WO02/34771, with PSORT analysis of the translation products.
Examples 30 to 117 describe GBS sequences not disclosed in WO02/34771, with PSORT analysis of the translation products.
Examples 118 to 152 describe GAS sequences not disclosed by Ferretti et al.
Example 153 describes a GAS sequence shorter than the Ferretti et al. sequence. Examples 154 to 173 describe GAS sequences longer than the Ferretti et al. sequences.
Examples 174 to 176 describe GAS sequences from the Ferretti et al. genome and which are predicted to be antigenic. Examples 177 to 674 describe GAS sequences.
Examples 675 to 686 describe GBS sequences.

GBS sequences are from a serotype V clinical strain isolated in Italy which expresses the R antigen (ISS/Rome/Italy collection, strain 2603 V/R; SEQ ID 1373). GAS sequences are from strain SF370 (ATCC 700294).

Various tests can be used to assess the in vivo immunogenicity of the proteins identified in the examples. For example, the proteins can be expressed recombinantly and used to screen patient sera by immunoblot. A positive reaction between the protein and patient serum indicates that the patient has previously mounted an immune response to the protein in question i.e. the protein is an immunogen. This method can also be used to identify immunodominant proteins. The mouse model used in the examples can also be used.

The recombinant protein can also be conveniently used to prepare antibodies e.g. in a mouse. These can be used for direct confirmation that a protein is located on the cell-surface. Labelled antibody (e.g. fluorescent labelling for FACS) can be incubated with intact bacteria and the presence of label on the bacterial surface confirms the location of the protein.

Details of experimental techniques which have been used for GBS coding sequence identification, expression, purification and characterisation are presented below:

### Sequence Analysis

Open reading frames (ORFs) within nucleotide sequences were predicted using the GLIMMER program [Salzberg et al. (1998) Nucleic Acids Res 26:544-8]. Where necessary, start codons were modified and corrected manually on the basis of the presence of ribosome-binding sites and promoter regions on the upstream DNA sequence.

Leader peptides within the ORFs were located using three different approaches: (i) PSORT [Nakai (1991) Bull. Inst. Chem. Res., Kyoto Univ. 69:269-291; Horton & Nakai (1996) Intellig. Syst. Mol. Biol. 4:109-115; Horton & Nakai (1997) Intellig. Syst. Mol. Biol. 5:147-152]; (ii) SignalP [Nielsen & Krogh (1998) in Proceedings of the Sixth International Conference on Intelligent Systems for Molecular Biology (ISMB 6), AAAI Press, Menlo Park, Calif., pp. 122-130; Nielsen et al. (1999) Protein Engineering 12:3-9; Nielsen et al. (1997). Int. J. Neural Sys. 8:581-599]; and (iii) visual inspection of the ORF sequences. Where a signal sequences is given a “possible site” value, the value represents the C-terminus residue of the signal peptide e.g. a “possible site” of 26 means that the signal sequence consists of amino acids 1-26.

Lipoprotein-specific signal peptides were located using three different approaches: (i) PSORT [see above]; (ii) the “prokaryotic membrane lipoprotein lipid attachment site” PROSITE motif [Hofmann et al. (1999) Nucleic Acids Res. 27:215-219; Bucher & Bairoch (1994) in Proceedings 2nd International Conference on Intelligent Systems for Molecular Biology (ISMB-94), AAAI Press, pages 53-61]; and (iii) the FINDPATTERNS program available in the GCG Wisconsin Package, using the pattern (M,L,V)x{9, 35} LxxCx.

Transmembrane domains were located using two approaches: (i) PSORT [see above]; (ii) TopPred [von Heijne (1992) J. Mol. Biol. 225:487-494].

LPXTG motifs, characteristic of cell-wall attached proteins in Gram-positive bacteria [Fischetti et al. (1990) Mol Microbiol 4:1603-5] were located with FINDPATTERNS using the pattern (L,I,V,M,Y,F)Px(T,A,S,G) (G,N,S,T,A,L).

RGD motifs, characteristic of cell-adhesion molecules [D'Souza et al. (1991) Trends Biochem Sci 16:246-50] were located using FINDPATTERNS.

Enzymes belonging to the glycolytic pathway were also selected as antigens, because these have been found experimentally expressed on the surface of Streptococci [e.g. Pancholi & Fischetti (1992) J Exp Med 176:415-26; Pancholi & Fischetti (1998) J Biol Chem 273:14503-15].

### Cloning, Expression and Purification of Proteins

GBS genes were cloned to facilitate expression in E. coli as two different types of fusion proteins:
a) proteins having a hexa-histidine tag at the amino-terminus (His-gbs)
b) proteins having a GST fusion partner at the amino-terminus (Gst-gbs)

Cloning was performed using the Gateway™ technology (Life Technologies), which is based on the site-specific recombination reactions that mediate integration and excision of phage lambda into and from the E. coli genome. A single cloning experiment included the following steps:
1—Amplification of GBS chromosomal DNA to obtain a PCR product coding for a single ORF flanked by attB recombination sites.
2—Insertion of the PCR product into a pDONR vector (containing attP sites) through a BP reaction (attB x attP sites). This reaction gives a so called ‘pEntry’ vector, which now contains attL sites flanking the insert.
3—Insertion of the GBS gene into E. coli expression vectors (pDestination vectors, containing attR sites) through a LR reaction between pEntry and pDestination plasmids (attL x attR sites).

### A) Chromosomal DNA Preparation

For chromosomal DNA preparation, GBS strain 2603 V/R (Istituto Superiore Sanità, Rome) was grown to exponential phase in 2 litres TH Broth (Difco) at 37° C., harvested by centrifugation, and dissolved in 40 ml TES (50 mM Tris pH 8, 5 mM EDTA pH 8, 20% sucrose). After addition of 2.5 ml lysozyme solution (25 mg/ml in TES) and 0.5 ml mutanolysin (Sigma M-9901, 25000 U/ml in H₂O), the suspension was incubated at 37° C. for 1 hour. 1 ml RNase (20 mg/ml) and 0.1 ml proteinase K (20 mg/ml) were added and incubation was continued for 30 min. at 37° C.

Cell lysis was obtained by adding 5 ml sarkosyl solution (10% N-laurylsarcosine in 250 mM EDTA pH 8.0), and incubating 1 hour at 37° C. with frequent inversion. After sequential extraction with phenol, phenol-chloroform and chloroform, DNA was precipitated with 0.3M sodium acetate pH 5.2 and 2 volumes of absolute ethanol. The DNA pellet was rinsed with 70% ethanol and dissolved in TE buffer (10 mM Tris-HCl, 1 mM EDTA, pH 8). DNA concentration was evaluated by OD₂₆₀.

### B) Oligonucleotide Design

Synthetic oligonucleotide primers were designed on the basis of the coding sequence of each ORF. The aim was to express the protein's extracellular region. Accordingly, predicted signal peptides were omitted (by deducing the 5′ end amplification primer sequence immediately downstream from the predicted leader sequence) and C-terminal cell-wall ancoring regions were removed (e.g. LPXTG motifs and downstream amino acids). Where additional nucleotides have been deleted, this is indicated by the suffix ‘d’ (e.g. ‘GBS352d’—see Table V). Conversely, a suffix ‘L’ refers to expression without these deletions. Deletions of C- or N-terminal residues were also sometimes made, as indicated by a ‘C’ or ‘N’ suffix.

The amino acid sequences of the expressed GBS proteins (including ‘d’ and ‘L’ forms etc.) are definitively defined by the sequences of the oligonucleotide primers given in Table H.

5′ tails of forward primers and 3′ tails of reverse primers included attB1 and attB2 sites respectively:

Forward primers:
5′-GGGGACAAGTTTGTACAAAAAAGCAGGCTCT-ORF in frame-3′
(the TCT sequence preceding the ORF was omitted
when the ORF's first coding triplet began with T).
Reverse primers:
5′-GGGGACCACTTTGTACAAGAAAGCTGGGTT-ORF reverse
complement-3′.

The number of nucleotides which hybridized to the sequence to be amplified depended on the melting temperature of the primers, which was determined as described by Breslauer et al. [PNAS USA (1986) 83:3746-50]. The average melting temperature of the selected oligos was 50-55° C. for the hybridizing region and 80-85° C. for the whole oligos.

### C) Amplification

The standard PCR protocol was as follows: 50 ng genomic DNA were used as template in the presence of 0.5 μM each primer, 200 μM each dNTP, 1.5 mM MgCl₂, 1× buffer minus Mg⁺⁺(Gibco-BRL) and 2 units of Taq DNA polymerase (Platinum Taq, Gibco-BRL) in a final volume of 100 μl. Each sample underwent a double-step of amplification: 5 cycles performed using as the hybridizing temperature 50° C., followed by 25 cycles at 68° C.

The standard cycles were as follows:
Denaturation: 94° C., 2 min
5 cycles: Denaturation: 94° C., 30 seconds
   Hybridization: 50° C., 50 seconds
   Elongation: 72° C., 1 min. or 2 min. and 40 sec.
25 cycles: Denaturation: 94° C., 30 seconds
   Hybridization: 68° C., 50 seconds
   Elongation: 72° C., 1 min. or 2 min. and 40 sec.

Elongation time was 1 minute for ORFs shorter than 2000 bp and 2:40 minutes for ORFs longer than 2000 bp. Amplifications were performed using a Gene Amp PCR system 9600 (Perkin Elmer).

To check amplification results, 2 μl of each PCR product were loaded onto 1-1.5 agarose gel and the size of amplified fragments was compared with DNA molecular weight standards (DNA marker IX Roche, 1 kb DNA ladder Biolabs).

Single band PCR products were purified by PEG precipitation: 300 μl of TE buffer and 200 μl of 30% PEG 8000/30 mM MgCl₂ were added to 100 μl PCR reaction. After vortexing, the DNA was centrifuged for 20 min at 10000 g, washed with 1 vol. 70% ethanol and the pellet dissolved in 30 μl TE. PCR products smaller than 350 bp were purified using a PCR purification Kit (Qiagen) and eluted with 30 μl of the provided elution buffer.

In order to evaluate the yield, 41 of the purified DNA were subjected to agarose gel electrophoresis and compared to titrated molecular weight standards.

D) Cloning of PCR Products into Expression Vectors

Cloning was performed following the Gateway™ technology's “one-tube protocol”, which consists of a two step reaction (BP and LR) for direct insertion of PCR products into expression vectors.

BP reaction (attB x attP sites): The reaction allowed insertion of the PCR product into a pDONR vector. The pDONR™ 201 vector we used contains the killer toxin gene ccdB between attP1 and attP2 sites to minimize background colonies lacking the PCR insert, and a selectable marker gene for kanamycin resistance. The reaction resulted in a so called pEntry vector, in which the GBS gene was located between attL1 and attL2 sites.

60 fmol of PCR product and 100 ng of pDONR™ vector were incubated with 2.5 μl of BP Clonase™ in a final volume of 12.5 μl for 4 hours at 25° C.

LR reaction (attL x attR sites): The reaction allowed the insertion of the GBS gene, now present in the pEntry vector, into E. coli expression vectors (pDestination vectors, containing attR sites). Two pDestination vectors were used (pDEST15 for N-terminal GST fusions—FIG. 86; and pDEST17-1 for N-terminal His-tagged fusions—FIG. 87). Both allow transcription of the ORF fusion coding mRNA under T7 RNA polymerase promoter [Studier et al (1990) Meth. Enzymol 185: 60ff].

To 5 μl of BP reaction were added 0.25 μl of 0.75 M NaCl, 100 ng of destination vector and 1.5 μl of LR Clonase™. The reaction was incubated at 25° C. for 2 hours and stopped with 1 μl of 1 mg/ml proteinase K solution at 37° C. for 15 min.

1 μl of the completed reaction was used to transform 50 μl electrocompetent BL21-SI™ cells (0.1 cm, 200 ohms, 25 μF). BL21-SI cells contain an integrated T7 RNA polymerase gene under the control of the salt-inducible prU promoter [Gowrishankar (1985) J. Bacteria 164:434ff]. After electroporation cells were diluted in 1 ml SOC medium (20 g/l bacto-tryptone, 5 g/l yeast extract, 0.58 g/l NaCl, 0.186 g/l KCl, 20 mM glucose, 10 mM MgCl₂) and incubated at 37° C. for 1 hour. 200 μl cells were plated onto LBON plates (Luria Broth medium without NaCl) containing 100 μg/ml ampicillin. Plates were then incubated for 16 hours at 37° C.

Entry clones: In order to allow the future preparation of Gateway compatible pEntry plasmids containing genes which might turn out of interest after immunological assays, 2.5 μl of BP reaction were incubated for 15 min in the presence of 3 μl 0.15 mg/ml proteinase K solution and then kept at −20° C. The reaction was in this way available to transform E. coli competent cells so as to produce Entry clones for future introduction of the genes in other Destination vectors.

### E) Protein Expression

Single colonies derived from the transformation of LR reactions were inoculated as small-scale cultures in 3 ml LBON 100 μg/ml ampicillin for overnight growth at 25° C. 50-200 μl of the culture was inoculated in 3 ml LBON/Amp to an initial OD600 of 0.1. The cultures were grown at 37° C. until OD600 0.4-0.6 and recombinant protein expression was induced by adding NaCl to a final concentration of 0.3 M. After 2 hour incubation the final OD was checked and the cultures were cooled on ice. 0.5 OD₆₀₀ of cells were harvested by centrifugation. The cell pellet was suspended in 50 μl of protein Loading Sample Buffer (50 mM TRIS-HCl pH 6.8, 0.5% w/v SDS, 2.5% v/v glycerin, 0.05% w/v Bromophenol Blue, 100 mM DTT) and incubated at 100° C. for 5 min. 10 μl of sample was analyzed by SDS-PAGE and Coomassie Blue staining to verify the presence of induced protein band.

### F) Purification of the Recombinant Proteins

Single colonies were inoculated in 25 ml LBON 100 μg/ml ampicillin and grown at 25° C. overnight. The overnight culture was inoculated in 500 ml LBON/amp and grown under shaking at 25° C. until OD₆₀₀ values of 0.4-0.6. Protein expression was then induced by adding NaCl to a final concentration of 0.3 M. After 3 hours incubation at 25° C. the final OD₆₀₀ was checked and the cultures were cooled on ice. After centrifugation at 6000 rpm (JA10 rotor, Beckman) for 20 min., the cell pellet was processed for purification or frozen at −20° C.

Proteins were purified in 1 of 3 ways depending on the fusion partner and the protein's solubility:

Purification of Soluble His-Tagged Proteins from E. Coli
1. Transfer pellets from −20° C. to ice bath and reconstitute each pellet with 10 ml B-PER™ solution (Bacterial-Protein Extraction Reagent, Pierce cat. 78266), 10 μl of a 100 mM MgCl₂ solution, 50 μl of DNAse I (Sigma D-4263, 100 Kunits in PBS) and 100 μl of 100 mg/ml lysozyme in PBS (Sigma L-7651, final concentration 1 mg/ml).
2. Transfer resuspended pellets in 50 ml centrifuge tubes and leave at room temperature for 30-40 minutes, vortexing 3-4 times.
3. Centrifuge 15-20 minutes at about 30-40000×g.
4. Prepare Poly-Prep (Bio-Rad) columns containing 1 ml of Fast Flow Ni-activated Chelating Sepharose (Pharmacia). Equilibrate with 50 mM phosphate buffer, 300 mM NaCl, pH 8.0.
5. Store the pellet at −20° C., and load the supernatant on to the columns.
6. Discard the flow through.
7. Wash with 10 ml 20 mM imidazole buffer, 50 mM phosphate, 300 mM NaCl, pH 8.0.
8. Elute the proteins bound to the columns with 4.5 ml (1.5 ml+1.5 ml+1.5 ml) 250 mM imidazole buffer, 50 mM phosphate, 300 mM NaCl, pH 8.0 and collect three fractions of ˜1.5 ml each. Add to each tube 15 μl DTT 200 mM (final concentration 2 mM).
9. Measure the protein concentration of the collected fractions with the Bradford method and analyse the proteins by SDS-PAGE.
10. Store the collected fractions at +4° C. while waiting for the results of the SDS-PAGE analysis.
11. For immunisation prepare 4-5 aliquots of 20-100 μg each in 0.5 ml in 40% glycerol. The dilution buffer is the above elution buffer, plus 2 mM DTT. Store the aliquots at −20° C. until immunisation.
   Purification of His-Tagged Proteins from Inclusion Bodies
1. Bacteria are collected from 500 ml cultures by centrifugation. If required store bacterial pellets at −20° C. Transfer the pellets from −20° C. to room temperature and reconstitute each pellet with 10 ml B-PER™ solution, 10 μl of a 100 mM MgCl₂ solution (final 1 mM), 50 μl of DNAse 1 equivalent to 100 Kunits units in PBS and 100 μl of a 100 mg/ml lysozime (Sigma L-7651) solution in PBS (equivalent to 10 mg, final concentration 1 mg/ml).
2. Transfer the resuspended pellets in 50 ml centrifuge tubes and let at room temperature for 30-40 minutes, vortexing 3-4 times.
3. Centrifuge 15 minutes at 30-4000×g and collect the pellets.
4. Dissolve the pellets with 50 mM TRIS-HCl, 1 mM TCEP {Tris(2-carboxyethyl)-phosphine hydrochloride, Pierce}, 6M guanidine hydrochloride, pH 8.5. Stir for ˜10 min. with a magnetic bar.
5. Centrifuge as described above, and collect the supernatant.
6. Prepare Poly-Prep (Bio-Rad) columns containing 1 ml of Fast Flow Ni-activated Chelating Sepharose (Pharmacia). Wash the columns twice with 5 ml of H₂O and equilibrate with 50 mM TRIS-HCl, 1 mM TCEP, 6M guanidine hydrochloride, pH 8.5.
7. Load the supernatants from step 5 onto the columns, and wash with 5 ml of 50 mM TRIS-HCl buffer, 1 mM TCEP, 6M urea, pH 8.5
8. Wash the columns with 10 ml of 20 mM imidazole, 50 mM TRIS-HCl, 6M urea, 1 mM TCEP, pH 8.5. Collect and set aside the first 5 ml for possible further controls.
9. Elute proteins bound to columns with 4.5 ml buffer containing 250 mM imidazole, 50 mM TRIS-HCl, 6M urea, 1 mM TCEP, pH 8.5. Add the elution buffer in three 1.5 ml aliquots, and collect the corresponding three fractions. Add to each fraction 15 μl DTT (final concentration 2 mM).
10. Measure eluted protein concentration with Bradford method and analyse proteins by SDS-PAGE.
11. Dialyse overnight the selected fraction against 50 mM Na phosphate buffer, pH 8.8, containing 10% glycerol, 0.5 M arginine, 5 mM reduced glutathione, 0.5 mM oxidized glutathione, 2 M urea.
12. Dialyse against 50 mM Na phosphate buffer, pH 8.8, containing 10% glycerol, 0.5 M arginine, 5 mM reduced glutathione, 0.5 mM oxidized glutathione.
13. Clarify the dialysed protein preparation by centrifugation and discard the non-soluble material and measure the protein concentration with the Bradford method.
14. For each protein destined to the immunization prepare 4-5 aliquot of 20-100 μg each in 0.5 ml after having adjusted the glycerol content up to 40%. Store the prepared aliquots at −20° C. until immunization.
   Purification of GST-Fusion Proteins from E. Coli
1. Bacteria are collected from 500 ml cultures by centrifugation. If required store bacterial pellets at −20° C. Transfer the pellets from −20° C. to room temperature and reconstitute each pellet with 10 ml B-PER™ solution, 10 μl of a 100 mM MgCl₂ solution (final 1 mM), 50 μl of DNAse I equivalent to 100 Kunits units in PBS and 100 μl of a 100 mg/ml lysozime (Sigma L-7651) solution in PBS (equivalent to 10 mg, final concentration 1 mg/ml).
2. Transfer the resuspended pellets in 50 ml centrifuge tubes and let at room temperature for 30-40 minutes, vortexing 3-4 times.
3. Centrifuge 15-20 minutes at about 30-40000×g.
4. Discard centrifugation pellets and load supernatants onto the chromatography columns, as follows.
5. Prepare Poly-Prep (Bio-Rad) columns containing 0.5 ml of Glutathione-Sepharose 4B resin. Wash the columns twice with 1 ml of H₂O and equilibrate with 10 ml PBS, pH 7.4.
6. Load supernatants on to the columns and discard the flow through.
7. Wash the columns with 10 ml PBS, pH 7.4.
8. Elute proteins bound to columns with 4.5 ml of 50 mM TRIS buffer, 10 mM reduced glutathione, pH 8.0, adding 1.5 ml+1.5 ml+1.5 ml and collecting the respective 3 fractions of ˜1.5 ml each.
9. Measure protein concentration of the fractions with the Bradford method and analyse the proteins by SDS-PAGE.
10. Store the collected fractions at +4° C. while waiting for the results of the SDS-PAGE analysis.
11. For each protein destined for immunisation prepare 4-5 aliquots of 20-100 ng each in 0.5 ml of 40% glycerol. The dilution buffer is 50 mM TRIS-HCl, 2 mM DTT, pH 8.0. Store the aliquots at −20° C. until immunisation.
   Immunisations with GBS Proteins

The purified proteins were used to immunise groups of four CD-1 mice intraperitoneally. 20 μg of each purified protein was injected in Freund's adjuvant at days 1, 21 & 35. Immune responses were monitored by using samples taken on day 0 & 49. Sera were analysed as pools of sera from each group of mice.

### FACScan Bacteria Binding Assay Procedure.

GBS serotype V 2603 V/R strain was plated on TSA blood agar plates and incubated overnight at 37° C. Bacterial colonies were collected from the plates using a sterile dracon swab and inoculated into 100 ml Todd Hewitt Broth. Bacterial growth was monitored every 30 minutes by following OD₆₀₀. Bacteria were grown until OD₆₀₀=0.7-0.8. The culture was centrifuged for 20 minutes at 5000 rpm. The supernatant was discarded and bacteria were washed once with PBS, resuspended in ¼ culture volume of PBS containing 0.05% paraformaldehyde, and incubated for 1 hour at 37° C. and then overnight at 4° C.

50 μl bacterial cells (OD₆₀₀ 0.1) were washed once with PBS and resuspended in 20 μl blocking serum (Newborn Calf Serum, Sigma) and incubated for 20 minutes at room temperature. The cells were then incubated with 100 μl diluted sera (1:200) in dilution buffer (20% Newborn Calf Serum 0.1% BSA in PBS) for 1 hour at 4° C. Cells were centrifuged at 5000 rpm, the supernatant aspirated and cells washed by adding 200 μl washing buffer (0.1% BSA in PBS). 50 μl R-Phicoerytrin conjugated F(ab)₂ goat anti-mouse, diluted 1:100 in dilution buffer, was added to each sample and incubated for 1 hour at 4° C. Cells were spun down by centrifugation at 5000 rpm and washed by adding 200 μl of washing buffer. The supernatant was aspirated and cells resuspended in 200 μl PBS. Samples were transferred to FACScan tubes and read. The condition for FACScan setting were: FL2 on; FSC—H threshold:54; FSC PMT Voltage: E 02; SSC PMT: 516; Amp. Gains 2.63; FL-2 PMT: 728. Compensation values: 0.

Samples were considered as positive if they had a Δ mean values>50 channel values.

### Whole Extracts Preparation

GBS serotype III COH1 strain and serotype V 2603 V/R strain cells were grown overnight in Todd Hewitt Broth. 1 ml of the culture was inoculated into 100 ml Todd Hewitt Broth. Bacterial growth was monitored every 30 minutes by following OD₆₀₀. The bacteria were grown until the OD reached 0.7-0.8. The culture was centrifuged for 20 minutes at 5000 rpm. The supernatant was discarded and bacteria were washed once with PBS, resuspended in 2 ml 50 mM Tris-HCl, pH 6.8 adding 400 units of Mutanolysin (Sigma-Aldrich) and incubated 3 hrs at 37° C. After 3 cycles of freeze/thaw, cellular debris were removed by centrifugation at 14000 g for 15 minutes and the protein concentration of the supernatant was measured by the Bio-Rad Protein assay, using BSA as a standard.

### Western Blotting

Purified proteins (50 ng) and total cell extracts (25 μg) derived from GBS serotype III COH1 strain and serotype V 2603 V/R strain were loaded on 12% or 15% SDS-PAGE and transferred to a nitrocellulose membrane. The transfer was performed for 1 hours at 100V at 4° C., in transferring buffer (25 mM Tris base, 192 mM glycine, 20% methanol). The membrane was saturated by overnight incubation at 4° C. in saturation buffer (5% skimmed milk, 0.1% Tween 20 in PBS). The membrane was incubated for 1 hour at room temperature with 1:1000 mouse sera diluted in saturation buffer. The membrane was washed twice with washing buffer (3% skimmed milk, 0.1% Tween 20 in PBS) and incubated for 1 hour with a 1:5000 dilution of horseradish peroxidase labelled anti-mouse Ig (Bio-Rad). The membrane was washed twice with 0.1% Tween 20 in PBS and developed with the Opti-4CN Substrate Kit (Bio-Rad). The reaction was stopped by adding water.

### In Vivo Passive Protection Assay in Neonatal Sepsis Mouse Model.

The immune sera collected from the CD1 immunized mice were tested in a mouse neonatal sepsis model to verify their protective efficacy in mice challenged with GBS serotype III. Newborn Balb/C littermates were randomly divided in two groups within 24 hrs from birth and injected subcutaneously with 25 μl of diluted sera (1:15) from immunized CD1 adult mice. One group received preimmune sera, the other received immune sera. Four hours later all pups were challenged with a 75% lethal dose of the GBS serotype III COH1 strain. The challenge dose obtained diluting a mid log phase culture was administered subcutaneously in 25 μl of saline. The number of pups surviving GBS infection was assessed every 12 hours for 4 days.

### Example 1

A DNA sequence was identified in S. agalactiae <SEQ ID 1> which encodes the amino acid sequence <SEQ ID 2>. Analysis of this protein sequence reveals the following:

Lipop: Possible site: −1 Crend: 7
McG: Discrim Score: −13.13
GvH: Signal Score (−7.5): −3.96
Possible site: 61
>>> Seems to have no N-terminal signal seq.
ALOM program count: 0 value: 5.57 threshold: 0.0
PERIPHERAL Likelihood = 5.57 34
modified ALOM score: −1.61
Final Results
bacterial cytoplasm --- Certainty = 0.2917(Affirmative) <succ>
bacterial membrane --- Certainty = 0.0000(Not Clear) <succ>
bacterial outside --- Certainty = 0.0000(Not Clear) <succ>

SEQ ID 2 is a longer variant of GBS36 from WO02/34771, with translation beginning at an upstream start codon. It is predicted to be peptidase, M23/M37 family.

### Example 2

A DNA sequence was identified in S. agalactiae <SEQ ID 3> which encodes the amino acid sequence <SEQ ID 4>. Analysis of this protein sequence reveals the following:

Lipop: Possible site: −1 Crend: 2
McG: Discrim Score: −1.67
GvH: Signal Score (−7.5): −1.3
Possible site: 32
>>> Seems to have no N-terminal signal seq.
ALOM program count: 10 value: −11.41 threshold: 0.0

| | | | |
|---|---|---|---|
| INTEGRAL | Likelihood = −11.41 | Transmembrane | 74-90 |
| | | | (65-96) |
| INTEGRAL | Likelihood = −8.39 | Transmembrane | 290-306 |
| | | | (286-310) |
| INTEGRAL | Likelihood = −6.58 | Transmembrane | 171-187 |
| | | | (166-192) |
| INTEGRAL | Likelihood = −5.63 | Transmembrane | 324-340 |
| | | | (317-343) |
| INTEGRAL | Likelihood = −5.52 | Transmembrane | 226-242 |
| | | | (223-245) |
| INTEGRAL | Likelihood = −4.99 | Transmembrane | 369-385 |
| | | | (361-393) |
| INTEGRAL | Likelihood = −3.82 | Transmembrane | 35-51 |
| | | | (34-59) |
| INTEGRAL | Likelihood = −2.87 | Transmembrane | 113-129 |
| | | | (107-130) |
| INTEGRAL | Likelihood = −2.81 | Transmembrane | 145-161 |
| | | | (145-163) |
| INTEGRAL | Likelihood = −2.18 | Transmembrane | 16-32 |
| | | | (16-33) |
| PERIPHERAL | Likelihood = 2.49 | 257 | |

modified ALOM score: 2.78
Final Results
bacterial membrane --- Certainty = 0.5564(Affirmative) <succ>
bacterial outside --- Certainty = 0.0000(Not Clear) <succ>
bacterial cytoplasm --- Certainty = 0.0000(Not Clear) <succ>

SEQ ID 4 is a longer variant of GBS570 from WO02/34771, with translation beginning at an upstream start codon. It is predicted to be MATE efflux family protein.

### Example 3

A DNA sequence was identified in S. agalactiae <SEQ ID 5> which encodes the amino acid sequence <SEQ ID 6>. Analysis of this protein sequence reveals the following:

Lipop: Possible site: −1 Crend: 6
McG: Discrim Score: 21.12
GvH: Signal Score (−7.5): −0.18
Possible site: 50
>>> Seems to have a cleavable N-term signal seq.
ALOM program count: 7 value: −11.15 threshold: 0.0

| | | | |
|---|---|---|---|
| INTEGRAL | Likelihood = −11.15 | Transmembrane | 161-177 |
| | | | (150-183) |
| INTEGRAL | Likelihood = −7.86 | Transmembrane | 83-99 |
| | | | (73-109) |
| INTEGRAL | Likelihood = −3.66 | Transmembrane | 209-225 |
| | | | (208-226) |
| INTEGRAL | Likelihood = −2.71 | Transmembrane | 266-282 |
| | | | (266-286) |
| INTEGRAL | Likelihood = −2.66 | Transmembrane | 54-70 |
| | | | (52-71) |
| INTEGRAL | Likelihood = −2.44 | Transmembrane | 287-303 |
| | | | (286-303) |
| INTEGRAL | Likelihood = −0.80 | Transmembrane | 115-131 |
| | | | (115-131) |
| PERIPHERAL | Likelihood = 3.45 | 242 | |

modified ALOM score: 2.73
Final Results
bacterial membrane --- Certainty = 0.5458(Affirmative) <succ>
bacterial outside --- Certainty = 0.0000(Not Clear) <succ>
bacterial cytoplasm --- Certainty = 0.0000(Not Clear) <succ>

SEQ ID 6 is a longer variant of GBS594 from WO02/34771, with translation beginning at an upstream start codon. It is predicted to be ribose ABC transporter, permease protein (rbsC).

### Example 4

A DNA sequence was identified in S. agalactiae <SEQ ID 7> which encodes the amino acid sequence <SEQ ID 8>. Analysis of this protein sequence reveals the following:

Lipop: Possible site: −1 Crend: 6
McG: Discrim Score: 8.16
GvH: Signal Score (−7.5): −4.32
Possible site: 35
>>> Seems to have an uncleavable N-term signal seq
ALOM program count: 11 value: −12.10 threshold: 0.0

| | | | |
|---|---|---|---|
| INTEGRAL | Likelihood = −12.10 | Transmembrane | 307-323 |
| | | | (303-331) |
| INTEGRAL | Likelihood = −10.03 | Transmembrane | 10-26 |
| | | | (1-36) |
| INTEGRAL | Likelihood = −9.82 | Transmembrane | 200-216 |
| | | | (192-221) |
| INTEGRAL | Likelihood = −8.60 | Transmembrane | 330-346 |
| | | | (324-353) |
| INTEGRAL | Likelihood = −7.48 | Transmembrane | 252-268 |
| | | | (250-276) |
| INTEGRAL | Likelihood = −5.52 | Transmembrane | 55-71 |
| | | | (51-74) |
| INTEGRAL | Likelihood = −5.31 | Transmembrane | 146-162 |
| | | | (143-166) |
| INTEGRAL | Likelihood = −4.88 | Transmembrane | 86-102 |
| | | | (85-103) |
| INTEGRAL | Likelihood = −4.78 | Transmembrane | 179-195 |
| | | | (172-198) |
| INTEGRAL | Likelihood = −3.13 | Transmembrane | 114-130 |
| | | | (114-130) |
| INTEGRAL | Likelihood = −2.97 | Transmembrane | 224-240 |
| | | | (224-245) |
| PERIPHERAL | Likelihood = 12.63 | 352 | |

modified ALOM score: 2.92
Final Results
bacterial membrane --- Certainty = 0.5840(Affirmative) <succ>
bacterial outside --- Certainty = 0.0000(Not Clear) <succ>
bacterial cytoplasm --- Certainty = 0.0000(Not Clear) <succ>

SEQ ID 8 is a longer variant of GBS538 from WO02/34771, with translation beginning at an upstream start codon. It is predicted to be glycosyl transferase, group 4 family protein.

### Example 5

A DNA sequence was identified in S. agalactiae <SEQ ID 9> which encodes the amino acid sequence <SEQ ID 10>. Analysis of this protein sequence reveals the following:

Lipop: Possible site: −1 Crend: 0
McG: Discrim Score: −11.97
GvH: Signal Score (−7.5): −3.79
Possible site: 36
>>> Seems to have no N-terminal signal seq.
ALOM program count: 5 value: −9.02 threshold: 0.0

| | | | |
|---|---|---|---|
| INTEGRAL | Likelihood = −9.02 | Transmembrane | 32-48 |
| | | | (22-50) |
| INTEGRAL | Likelihood = −5.68 | Transmembrane | 148-164 |
| | | | (145-167) |
| INTEGRAL | Likelihood = −4.30 | Transmembrane | 72-88 |
| | | | (66-94) |
| INTEGRAL | Likelihood = −3.72 | Transmembrane | 129-145 |
| | | | (129-145) |
| INTEGRAL | Likelihood = −3.19 | Transmembrane | 181-197 |
| | | | (180-199) |
| PERIPHERAL | Likelihood = 2.17 | 55 | |

modified ALOM score: 2.30
Final Results
bacterial membrane --- Certainty = 0.4609(Affirmative) <succ>
bacterial outside --- Certainty = 0.0000(Not Clear) <succ>
bacterial cytoplasm --- Certainty = 0.0000(Not Clear) <succ>

SEQ ID 10 is a longer variant of GBS590 from WO02/34771, with translation beginning at an upstream start codon. It is predicted to be amino acid ABC transporter, permease protein.

### Example 6

A DNA sequence was identified in S. agalactiae <SEQ ID 11> which encodes the amino acid sequence <SEQ ID 12>. Analysis of this protein sequence reveals the following:

Lipop: Possible site: −1 Crend: 7
McG: Discrim Score: −3.19
GvH: Signal Score (−7.5): −4.17
Possible site: 17
>>> Seems to have no N-terminal signal seq.
ALOM program count: 4 value: −10.40 threshold: 0.0

| | | | |
|---|---|---|---|
| INTEGRAL | Likelihood = −10.40 | Transmembrane | 78-94 |
| | | | (71-104) |
| INTEGRAL | Likelihood = −7.86 | Transmembrane | 117-133 |
| | | | (115-139) |
| INTEGRAL | Likelihood = −6.90 | Transmembrane | 48-64 |
| | | | (43-66) |
| INTEGRAL | Likelihood = −5.52 | Transmembrane | 147-163 |
| | | | (137-164) |
| PERIPHERAL | Likelihood = 11.83 | 12 | |

modified ALOM score: 2.58
Final Results
bacterial membrane --- Certainty = 0.5161(Affirmative) <succ>
bacterial outside --- Certainty = 0.0000(Not Clear) <succ>
bacterial cytoplasm --- Certainty = 0.0000(Not Clear) <succ>

SEQ ID 12 is a longer variant of GBS566 from WO02/34771, with translation beginning at an upstream start codon.

### Example 7

A DNA sequence was identified in S. agalactiae <SEQ ID 13> which encodes the amino acid sequence <SEQ ID 14>. Analysis of this protein sequence reveals the following:

Lipop: Possible site: −1 Crend: 10
McG: Discrim Score: −5.99
GvH: Signal Score (−7.5): −4.37
Possible site: 27
>>> Seems to have no N-terminal signal seq.
ALOM program count: 2 value: −2.18 threshold: 0.0

| | | | |
|---|---|---|---|
| INTEGRAL | Likelihood = −2.18 | Transmembrane | 110-126 |
| | | | (110-127) |
| INTEGRAL | Likelihood = −1.06 | Transmembrane | 136-152 |
| | | | (136-152) |
| PERIPHERAL | Likelihood = 1.32 | 49 | |

modified ALOM score: 0.94
Final Results
bacterial membrane --- Certainty = 0.1871(Affirmative) <succ>
bacterial outside --- Certainty = 0.0000(Not Clear) <succ>
bacterial cytoplasm --- Certainty = 0.0000(Not Clear) <succ>

SEQ ID 14 is a longer variant of GBS374 from WO02/34771, with translation beginning at an upstream start codon. It is predicted to be enoyl-CoA hydratase/isomerase family protein.

### Example 8

A DNA sequence was identified in S. agalactiae <SEQ ID 15> which encodes the amino acid sequence <SEQ ID 16>. Analysis of this protein sequence reveals the following:

Lipop: Possible site: −1 Crend: 10
McG: Discrim Score: 1.67
GvH: Signal Score (−7.5): −5.85
Possible site: 38
>>> Seems to have an uncleavable N-term signal seq
ALOM program count: 5 value: −8.65 threshold: 0.0

| | | | |
|---|---|---|---|
| INTEGRAL | Likelihood = −8.65 | Transmembrane | 163-179 |
| | | | (154-182) |
| INTEGRAL | Likelihood = −5.15 | Transmembrane | 24-40 |
| | | | (20-45) |
| INTEGRAL | Likelihood = −4.88 | Transmembrane | 142-158 |
| | | | (132-162) |
| INTEGRAL | Likelihood = −1.49 | Transmembrane | 250-266 |
| | | | (250-269) |
| INTEGRAL | Likelihood = −1.33 | Transmembrane | 60-76 |
| | | | (60-76) |
| PERIPHERAL | Likelihood = 3.02 | 482 | |

modified ALOM score: 2.23
Final Results
bacterial membrane --- Certainty = 0.4461(Affirmative) <succ>
bacterial outside --- Certainty = 0.0000(Not Clear) <succ>
bacterial cytoplasm --- Certainty = 0.0000(Not Clear) <succ>

SEQ ID 16 is a longer variant of GBS540 from WO02/34771, with translation beginning at an upstream start codon. It is predicted to be ABC transporter, ATP-binding/permease protein.

### Example 9

A DNA sequence was identified in S. agalactiae <SEQ ID 17> which encodes the amino acid sequence <SEQ ID 18>. Analysis of this protein sequence reveals the following:

Lipop: Possible site: −1 Crend: 0
McG: Discrim Score: −4.80
GvH: Signal Score (−7.5): −3.64
Possible site: 36
>>> Seems to have no N-terminal signal seq.
ALOM program count: 1 value: −3.19 threshold: 0.0

| | | | |
|---|---|---|---|
| INTEGRAL | Likelihood = −3.19 | Transmembrane | 294-310 |
| | | | (293-310) |
| PERIPHERAL | Likelihood = 0.53 | 97 | |

modified ALOM score: 1.14
Final Results
bacterial membrane --- Certainty = 0.2275(Affirmative) <succ>
bacterial outside --- Certainty = 0.0000(Not Clear) <succ>
bacterial cytoplasm --- Certainty = 0.0000(Not Clear) <succ>

SEQ ID 18 is a longer variant of GBS73 from WO02/34771, with translation beginning at an upstream start codon. It is predicted to be cell division protein FtsA (ftsA).

### Example 10

A DNA sequence was identified in S. agalactiae <SEQ ID 19> which encodes the amino acid sequence <SEQ ID 20>. Analysis of this protein sequence reveals the following:

Lipop: Possible site: −1 Crend: 0
McG: Discrim Score: 10.14
GvH: Signal Score (−7.5): −2.52
Possible site: 48
>>> Seems to have an uncleavable N-term signal seq
ALOM program count: 2 value: −15.28 threshold: 0.0

| | | | |
|---|---|---|---|
| INTEGRAL | Likelihood = −15.28 | Transmembrane | 234-250 |
| | | | (229-257) |
| INTEGRAL | Likelihood = −0.11 | Transmembrane | 3-19 |
| | | | (3-20) |
| PERIPHERAL | Likelihood = 2.76 | 84 | |

modified ALOM score: 3.56
Final Results
bacterial membrane --- Certainty = 0.7114(Affirmative) <succ>
bacterial outside --- Certainty = 0.0000(Not Clear) <succ>
bacterial cytoplasm --- Certainty = 0.0000(Not Clear) <succ>

SEQ ID 20 is a longer variant of GBS208 from WO02/34771, with translation beginning at an upstream start codon. It is predicted to be sortase family protein.

### Example 11

A DNA sequence was identified in S. agalactiae <SEQ ID 21> which encodes the amino acid sequence <SEQ ID 22>. Analysis of this protein sequence reveals the following:

Lipop: Possible site: −1 Crend: 10
McG: Discrim Score: −24.35
GvH: Signal Score (−7.5): −10.27
Possible site: 50
>>> Seems to have no N-terminal signal seq.
ALOM program count: 1 value: −4.30 threshold: 0.0

| | | | |
|---|---|---|---|
| INTEGRAL | Likelihood = −4.30 | Transmembrane | 1039-1055 |
| | | | (1037-1056) |
| PERIPHERAL | Likelihood = 3.71 | 647 | |

modified ALOM score: 1.36
Final Results
bacterial membrane --- Certainty = 0.2720(Affirmative) <succ>
bacterial outside --- Certainty = 0.0000(Not Clear) <succ>
bacterial cytoplasm --- Certainty = 0.0000(Not Clear) <succ>

SEQ ID 22 is a longer variant of GBS195 from WO02/34771, with translation beginning at an upstream start codon.

### Example 12

A DNA sequence was identified in S. agalactiae <SEQ ID 23> which encodes the amino acid sequence <SEQ ID 24>. Analysis of this protein sequence reveals the following:

Lipop: Possible site: −1 Crend: 0
McG: Discrim Score: 5.21
GvH: Signal Score (−7.5): −7.17
Possible site: 13
>>> Seems to have an uncleavable N-term signal seq
ALOM program count: 1 value: −2.28 threshold: 0.0

| | | | |
|---|---|---|---|
| INTEGRAL | Likelihood = −2.28 | Transmembrane | 182-198 |
| | | | (180-198) |
| PERIPHERAL | Likelihood = 4.35 | 133 | |

modified ALOM score: 0.96
Final Results
bacterial membrane --- Certainty = 0.1914(Affirmative) <succ>
bacterial outside --- Certainty = 0.0000(Not Clear) <succ>
bacterial cytoplasm --- Certainty = 0.0000(Not Clear) <succ>

SEQ ID 24 is a longer variant of GBS259 from WO02/34771, with translation beginning at an upstream start codon.

### Example 13

A DNA sequence was identified in S. agalactiae <SEQ ID 25> which encodes the amino acid sequence <SEQ ID 26>. Analysis of this protein sequence reveals the following:

Lipop: Possible site: −1 Crend: 10
McG: Discrim Score: 20.16
GvH: Signal Score (−7.5): −2.08
Possible site: 30
>>> Seems to have a cleavable N-term signal seq.
ALOM program count: 0 value: 10.45 threshold: 0.0
PERIPHERAL Likelihood = 10.45 32
modified ALOM score: −2.59
Final Results
bacterial outside --- Certainty = 0.3000(Affirmative) <succ>
bacterial membrane --- Certainty = 0.0000(Not Clear) <succ>
bacterial cytoplasm --- Certainty = 0.0000(Not Clear) <succ>

SEQ ID 26 is a longer variant of GBS168 from WO02/34771, with translation beginning at an upstream start codon.

### Example 14

A DNA sequence was identified in S. agalactiae <SEQ ID 27> which encodes the amino acid sequence <SEQ ID 28>. Analysis of this protein sequence reveals the following:

Lipop: Possible site: −1 Crend: 3
McG: Discrim Score: 10.84
GvH: Signal Score (−7.5): −2.23
Possible site: 20
>>> Seems to have a cleavable N-term signal seq.
ALOM program count: 3 value: −11.94 threshold: 0.0

| | | | |
|---|---|---|---|
| INTEGRAL | Likelihood = −11.94 | Transmembrane | 127-143 |
| | | | (118-151) |
| INTEGRAL | Likelihood = −11.09 | Transmembrane | 185-201 |
| | | | (178-204) |
| INTEGRAL | Likelihood = −4.94 | Transmembrane | 90-106 |
| | | | (89-110) |
| PERIPHERAL | Likelihood = 2.33 | 23 | |

modified ALOM score: 2.89
Final Results
bacterial membrane --- Certainty = 0.5776(Affirmative) <succ>
bacterial outside --- Certainty = 0.0000(Not Clear) <succ>
bacterial cytoplasm --- Certainty = 0.0000(Not Clear) <succ>

SEQ ID 28 is a longer variant of GBS167 from WO02/34771, with translation beginning at an upstream start codon.

### Example 15

A DNA sequence was identified in S. agalactiae <SEQ ID 29> which encodes the amino acid sequence <SEQ ID 30>. Analysis of this protein sequence reveals the following:

Lipop: Possible site: 21 Crend: 3
McG: Discrim Score: 11.16
GvH: Signal Score (−7.5): −1.96
Possible site: 23
>>> May be a lipoprotein
ALOM program count: 0 value: 8.96 threshold: 0.0
PERIPHERAL Likelihood = 8.96 67
modified ALOM score: −2.29
Final Results
bacterial membrane --- Certainty = 0.0000(Not Clear) <succ>
bacterial outside --- Certainty = 0.0000(Not Clear) <succ>
bacterial cytoplasm --- Certainty = 0.0000(Not Clear) <succ>

SEQ ID 30 is a longer variant of GBS225 from WO02/34771, with translation beginning at an upstream start codon. It is predicted to be amino acid ABC transporter, amino acid-binding protein.

### Example 16

A DNA sequence was identified in S. agalactiae <SEQ ID 31> which encodes the amino acid sequence <SEQ ID 32>. Analysis of this protein sequence reveals the following:

Lipop: Possible site: −1 Crend: 0
McG: Discrim Score: 7.64
GvH: Signal Score (−7.5): −1.53
Possible site: 18
>>> Seems to have a cleavable N-term signal seq.
ALOM program count: 8 value: −7.75 threshold: 0.0

| | | | |
|---|---|---|---|
| INTEGRAL | Likelihood = −7.75 | Transmembrane | 156-172 |
| | | | (153-175) |
| INTEGRAL | Likelihood = −7.38 | Transmembrane | 69-85 |
| | | | (66-93) |
| INTEGRAL | Likelihood = −5.47 | Transmembrane | 285-301 |
| | | | (280-308) |
| INTEGRAL | Likelihood = −4.09 | Transmembrane | 103-119 |
| | | | (102-120) |
| INTEGRAL | Likelihood = −3.24 | Transmembrane | 39-55 |
| | | | (39-55) |
| INTEGRAL | Likelihood = −1.91 | Transmembrane | 254-270 |
| | | | (254-271) |
| INTEGRAL | Likelihood = −1.33 | Transmembrane | 230-246 |
| | | | (229-247) |
| INTEGRAL | Likelihood = −0.00 | Transmembrane | 205-221 |
| | | | (205-221) |
| PERIPHERAL | Likelihood = 3.34 | 135 | |

modified ALOM score: 2.05
Final Results
bacterial membrane --- Certainty = 0.4100(Affirmative) <succ>
bacterial outside --- Certainty = 0.0000(Not Clear) <succ>
bacterial cytoplasm --- Certainty = 0.0000(Not Clear) <succ>

SEQ ID 32 is a longer variant of GBS507 from WO02/34771, with translation beginning at an upstream start codon. It is predicted to be a sugar ABC transporter, permease protein.

### Example 17

A DNA sequence was identified in S. agalactiae <SEQ ID 33> which encodes the amino acid sequence <SEQ ID 34>. Analysis of this protein sequence reveals the following:

Lipop: Possible site: −1 Crend: 8
McG: Discrim Score: −6.80
GvH: Signal Score (−7.5): −4.75
Possible site: 59
>>> Seems to have no N-terminal signal seq.
ALOM program count: 1 value: −0.22 threshold: 0.0

| | | | |
|---|---|---|---|
| INTEGRAL | Likelihood = −0.22 | Transmembrane | 93-109 |
| | | | (93-109) |
| PERIPHERAL | Likelihood = 0.47 | 49 | |

modified ALOM score: 0.54
Final Results
bacterial membrane --- Certainty = 0.1086(Affirmative) <succ>
bacterial outside --- Certainty = 0.0000(Not Clear) <succ>
bacterial cytoplasm --- Certainty = 0.0000(Not Clear) <succ>

SEQ ID 34 is a longer variant of GBS390 from WO02/34771, with translation beginning at an upstream start codon. It is predicted to be lipoate-protein ligase A family protein.

### Example 18

A DNA sequence was identified in S. agalactiae <SEQ ID 35> which encodes the amino acid sequence <SEQ ID 36>. Analysis of this protein sequence reveals the following:

Lipop: Possible site: −1 Crend: 10
McG: Discrim Score: 6.20
GvH: Signal Score (−7.5): −4.59
Possible site: 15
>>> Seems to have an uncleavable N-term signal seq
ALOM program count: 10 value: −12.10 threshold: 0.0

| | | | |
|---|---|---|---|
| INTEGRAL | Likelihood = −12.10 | Transmembrane | 428-444 |
| | | | (420-449) |
| INTEGRAL | Likelihood = −8.92 | Transmembrane | 146-162 |
| | | | (144-171) |
| INTEGRAL | Likelihood = −8.86 | Transmembrane | 401-417 |
| | | | (399-425) |
| INTEGRAL | Likelihood = −7.91 | Transmembrane | 296-312 |
| | | | (290-315) |
| INTEGRAL | Likelihood = −6.42 | Transmembrane | 377-393 |
| | | | (371-395) |
| INTEGRAL | Likelihood = −5.31 | Transmembrane | 347-363 |
| | | | (344-364) |
| INTEGRAL | Likelihood = −4.57 | Transmembrane | 53-69 |
| | | | (51-71) |
| INTEGRAL | Likelihood = −3.24 | Transmembrane | 169-185 |
| | | | (168-195) |
| INTEGRAL | Likelihood = −1.33 | Transmembrane | 221-237 |
| | | | (221-237) |
| INTEGRAL | Likelihood = −0.59 | Transmembrane | 98-114 |
| | | | (98-114) |
| PERIPHERAL | Likelihood = 0.85 | 17 | |

modified ALOM score: 2.92
Final Results
bacterial membrane --- Certainty = 0.5840(Affirmative) <succ>
bacterial outside --- Certainty = 0.0000(Not Clear) <succ>
bacterial cytoplasm --- Certainty = 0.0000(Not Clear) <succ>

SEQ ID 36 is a longer variant of GBS638 from WO02/34771, with translation beginning at an upstream start codon. It is predicted to be potassium uptake protein.

### Example 19

A DNA sequence was identified in S. agalactiae <SEQ ID 37> which encodes the amino acid sequence <SEQ ID 38>. Analysis of this protein sequence reveals the following:

Lipop: Possible site: −1 Crend: 8
McG: Discrim Score: −5.88
GvH: Signal Score (−7.5): −2.86
Possible site: 61
>>> Seems to have no N-terminal signal seq.
ALOM program count: 0 value: 1.43 threshold: 0.0
PERIPHERAL Likelihood = 1.43 49
modified ALOM score: −0.79
Final Results
bacterial cytoplasm --- Certainty = 0.1249(Affirmative) <succ>
bacterial membrane --- Certainty = 0.0000(Not Clear) <succ>
bacterial outside --- Certainty = 0.0000(Not Clear) <succ>

SEQ ID 38 is a longer variant of GBS308 from WO02/34771, with translation beginning at an upstream start codon.

### Example 20

A DNA sequence was identified in S. agalactiae <SEQ ID 39> which encodes the amino acid sequence <SEQ ID 40>. Analysis of this protein sequence reveals the following:

Lipop: Possible site: −1 Crend: 10
McG: Discrim Score: −5.56
GvH: Signal Score (−7.5): −4.41
Possible site: 28
>>> Seems to have no N-terminal signal seq.
ALOM program count: 0 value: 2.49 threshold: 0.0
PERIPHERAL Likelihood = 2.49 225
modified ALOM score: −1.00
Final Results
bacterial cytoplasm --- Certainty = 0.1495(Affirmative) <succ>
bacterial membrane --- Certainty = 0.0000(Not Clear) <succ>
bacterial outside --- Certainty = 0.0000(Not Clear) <succ>

SEQ ID 40 is a longer variant of GBS381 from WO02/34771, with translation beginning at an upstream start codon. It is predicted to be homocysteine S-methyltransferase MmuM.

### Example 21

A DNA sequence was identified in S. agalactiae <SEQ ID 41> which encodes the amino acid sequence <SEQ ID 42>. Analysis of this protein sequence reveals the following:

Lipop: Possible site: −1 Crend: 0
McG: Discrim Score: −0.04
GvH: Signal Score (−7.5): −3.64
Possible site: 46
>>> Seems to have no N-terminal signal seq.
ALOM program count: 0 value: 1.64 threshold: 0.0
PERIPHERAL Likelihood = 1.64 46
modified ALOM score: −0.83
Final Results
bacterial cytoplasm --- Certainty = 0.0236(Affirmative) <succ>
bacterial membrane --- Certainty = 0.0000(Not Clear) <succ>
bacterial outside --- Certainty = 0.0000(Not Clear) <succ>

SEQ ID 42 is a longer variant of GBS647 from WO02/34771, with translation beginning at an upstream start codon. It is predicted to be 4-diphosphocytidyl-2C-methyl-D-erythritol synthase.

### Example 22

A DNA sequence was identified in S. agalactiae <SEQ ID 43> which encodes the amino acid sequence <SEQ ID 44>. Analysis of this protein sequence reveals the following:

Lipop: Possible site: −1 Crend: 2
McG: Discrim Score: −0.72
GvH: Signal Score (−7.5): −3.51
Possible site: 61
>>> Seems to have no N-terminal signal seq.
ALOM program count: 0 value: 8.43 threshold: 0.0
PERIPHERAL Likelihood = 8.43 109
modified ALOM score: −2.19
Final Results
bacterial cytoplasm --- Certainty = 0.0347(Affirmative) <succ>
bacterial membrane --- Certainty = 0.0000(Not Clear) <succ>
bacterial outside --- Certainty = 0.0000(Not Clear) <succ>

SEQ ID 44 is a longer variant of GBS245 from WO02/34771, with translation beginning at an upstream start codon. It is predicted to be dephospho-CoA kinase.

### Example 23

A DNA sequence was identified in S. agalactiae <SEQ ID 45> which encodes the amino acid sequence <SEQ ID 46>. Analysis of this protein sequence reveals the following:

Lipop: Possible site: −1 Crend: 10
McG: Discrim Score: −17.55
GvH: Signal Score (−7.5): −4.5
Possible site: 27
>>> Seems to have no N-terminal signal seq.
ALOM program count: 0 value: 7.21 threshold: 0.0
PERIPHERAL Likelihood = 7.21 257
modified ALOM score: −1.94
Final Results
bacterial cytoplasm --- Certainty = 0.3910(Affirmative) <succ>
bacterial membrane --- Certainty = 0.0000(Not Clear) <succ>
bacterial outside --- Certainty = 0.0000(Not Clear) <succ>

SEQ ID 46 is a longer variant of GBS186 from WO02/34771, with translation beginning at an upstream start codon. It is predicted to be peptide ABC transporter, peptide-binding protein.

### Example 24

A DNA sequence was identified in S. agalactiae <SEQ ID 47> which encodes the amino acid sequence <SEQ ID 48>. Analysis of this protein sequence reveals the following:

Lipop: Possible site: −1 Crend: 9
McG: Discrim Score: −24.57
GvH: Signal Score (−7.5): −6.25
Possible site: 56
>>> Seems to have no N-terminal signal seq.
ALOM program count: 1 value: −4.14 threshold: 0.0

| | | | |
|---|---|---|---|
| INTEGRAL | Likelihood = −4.14 | Transmembrane | 115-131 |
| | | | (114-132) |
| PERIPHERAL | Likelihood = 7.74 | 162 | |

modified ALOM score: 1.33
Final Results
bacterial membrane --- Certainty = 0.2657(Affirmative) <succ>
bacterial outside --- Certainty = 0.0000(Not Clear) <succ>
bacterial cytoplasm --- Certainty = 0.0000(Not Clear) <succ>

SEQ ID 48 is a longer variant of GBS383 from WO02/34771, with translation beginning at an upstream start codon. It is predicted to be integrase/recombinase, phage integrase family.

### Example 25

A DNA sequence was identified in S. agalactiae <SEQ ID 49> which encodes the amino acid sequence <SEQ ID 50>. Analysis of this protein sequence reveals the following:

Lipop: Possible site: −1 Crend: 7
McG: Discrim Score: 13.50
GvH: Signal Score (−7.5): 0.21
Possible site: 52
>>> Seems to have a cleavable N-term signal seq.
ALOM program count: 7 value: −8.70 threshold: 0.0

| | | | |
|---|---|---|---|
| INTEGRAL | Likelihood = −8.70 | Transmembrane | 245-261 |
| | | | (241-269) |
| INTEGRAL | Likelihood = −6.42 | Transmembrane | 89-105 |
| | | | (84-119) |
| INTEGRAL | Likelihood = −6.26 | Transmembrane | 173-189 |
| | | | (169-194) |
| INTEGRAL | Likelihood = −5.47 | Transmembrane | 269-285 |
| | | | (268-289) |
| INTEGRAL | Likelihood = −4.35 | Transmembrane | 107-123 |
| | | | (106-126) |
| INTEGRAL | Likelihood = −3.29 | Transmembrane | 136-152 |
| | | | (135-153) |
| INTEGRAL | Likelihood = −2.76 | Transmembrane | 200-216 |
| | | | (200-219) |
| PERIPHERAL | Likelihood = 4.40 | 217 | |

modified ALOM score: 2.24
Final Results
bacterial membrane --- Certainty = 0.4482(Affirmative) <succ>
bacterial outside --- Certainty = 0.0000(Not Clear) <succ>
bacterial cytoplasm --- Certainty = 0.0000(Not Clear) <succ>

SEQ ID 50 is a longer variant of GBS517 from WO02/34771, with translation beginning at an upstream start codon. It is predicted to be membrane protein.

### Example 26

A DNA sequence was identified in S. agalactiae <SEQ ID 51> which encodes the amino acid sequence <SEQ ID 52>. Analysis of this protein sequence reveals the following:

Lipop: Possible site: −1 Crend: 9
McG: Discrim Score: −8.86
GvH: Signal Score (−7.5): −4.55
Possible site: 40
>>> Seems to have no N-terminal signal seq.
ALOM program count: 0 value: 2.81 threshold: 0.0
PERIPHERAL Likelihood = 2.81 66
modified ALOM score: −1.06
Final Results
bacterial cytoplasm --- Certainty = 0.2181(Affirmative) <succ>
bacterial membrane --- Certainty = 0.0000(Not Clear) <succ>
bacterial outside --- Certainty = 0.0000(Not Clear) <succ>

SEQ ID 52 is a longer variant of GBS648 from WO02/34771, with translation beginning at an upstream start codon.

### Example 27

A DNA sequence was identified in S. agalactiae <SEQ ID 53> which encodes the amino acid sequence <SEQ ID 54>. Analysis of this protein sequence reveals the following:

Lipop: Possible site: −1 Crend: 0
McG: Discrim Score: 9.74
GvH: Signal Score (−7.5): −3.42
Possible site: 21
>>> Seems to have an uncleavable N-term signal seq
ALOM program count: 2 value: −3.61 threshold: 0.0

| | | | |
|---|---|---|---|
| INTEGRAL | Likelihood = −3.61 | Transmembrane | 5-21 (1-22) |
| INTEGRAL | Likelihood = −1.97 | Transmembrane | 34-50 (33-53) |
| PERIPHERAL | Likelihood = 1.01 | 205 | |

modified ALOM score: 1.22
Final Results
bacterial membrane --- Certainty = 0.2444(Affirmative) <succ>
bacterial outside --- Certainty = 0.0000(Not Clear) <succ>
bacterial cytoplasm --- Certainty = 0.0000(Not Clear) <succ>

SEQ ID 54 is a longer variant of GBS216 from WO02/34771, with translation beginning at an upstream start codon. It is predicted to be FtsK/SpoIIIE family protein.

### Example 28

A DNA sequence was identified in S. agalactiae <SEQ ID 55> which encodes the amino acid sequence <SEQ ID 56>. Analysis of this protein sequence reveals the following:

Lipop: Possible site: −1 Crend: 3
McG: Discrim Score: −17.31
GvH: Signal Score (−7.5): −1.85
Possible site: 58
>>> Seems to have no N-terminal signal seq.
ALOM program count: 1 value: −4.94 threshold: 0.0

| | | | |
|---|---|---|---|
| INTEGRAL | Likelihood = −4.94 | Transmembrane | 801-817 |
| | | | (799-821) |
| PERIPHERAL | Likelihood = 4.29 | 32 | |

modified ALOM score: 1.49
Final Results
bacterial membrane --- Certainty = 0.2975(Affirmative) <succ>
bacterial outside --- Certainty = 0.0000(Not Clear) <succ>
bacterial cytoplasm --- Certainty = 0.0000(Not Clear) <succ>

SEQ ID 56 is a longer variant of GBS191 from WO02/34771, with translation beginning at an upstream start codon. It is predicted to be cell wall surface anchor family protein.

### Example 29

A DNA sequence was identified in S. agalactiae <SEQ ID 57> which encodes the amino acid sequence <SEQ ID 58>. Analysis of this protein sequence reveals the following:

Lipop: Possible site: −1 Crend: 8
McG: Discrim Score: −5.16
GvH: Signal Score (−7.5): −2.17
Possible site: 42
>>> Seems to have no N-terminal signal seq.
ALOM program count: 1 value: −0.16 threshold: 0.0

| | | | |
|---|---|---|---|
| INTEGRAL | Likelihood = −0.16 | Transmembrane | 34-50 (34-50) |
| PERIPHERAL | Likelihood = 4.14 | 16 | |

modified ALOM score: 0.53
Final Results
bacterial membrane --- Certainty = 0.1065(Affirmative) <succ>
bacterial outside --- Certainty = 0.0000(Not Clear) <succ>
bacterial cytoplasm --- Certainty = 0.0000(Not Clear) <succ>

SEQ ID 58 is a longer variant of GBS394 from WO02/34771, with translation beginning at an upstream start codon.

### Example 30

A DNA sequence was identified in S. agalactiae <SEQ ID 59> which encodes the amino acid sequence <SEQ ID 60>. Analysis of this protein sequence reveals the following:

Lipop: Possible site: −1 Crend: 0
McG: Discrim Score: 0.49
GvH: Signal Score (−7.5): −6.93
Possible site: 13
>>> Seems to have an uncleavable N-term signal seq
ALOM program count: 0 value: 0.47 threshold: 0.0
PERIPHERAL Likelihood = 0.47 60
modified ALOM score: −0.59
Final Results
bacterial membrane --- Certainty = 0.0000(Not Clear) <succ>
bacterial outside --- Certainty = 0.0000(Not Clear) <succ>
bacterial cytoplasm --- Certainty = 0.0000(Not Clear) <succ>

SEQ ID 60 is predicted to be phosphoribosylaminoimidazole carboxylase, catalytic subunit (purE).

### Example 31

A DNA sequence was identified in S. agalactiae <SEQ ID 61> which encodes the amino acid sequence <SEQ ID 62>. Analysis of this protein sequence reveals the following:

Lipop: Possible site: −1 Crend: 4
McG: Discrim Score: −13.80
GvH: Signal Score (−7.5): −7.02
Possible site: 37
>>> Seems to have no N-terminal signal seq.
ALOM program count: 0 value: 11.03 threshold: 0.0
PERIPHERAL Likelihood = 11.03 42
modified ALOM score: −2.71
Final Results
bacterial cytoplasm --- Certainty = 0.3665(Affirmative) <succ>
bacterial membrane --- Certainty = 0.0000(Not Clear) <succ>
bacterial outside --- Certainty = 0.0000(Not Clear) <succ>

SEQ ID 62 is predicted to be transcriptional regulator ComX1.

### Example 32

A DNA sequence was identified in S. agalactiae <SEQ ID 63> which encodes the amino acid sequence <SEQ ID 64>. Analysis of this protein sequence reveals the following:

Lipop: Possible site: −1 Crend: 7
McG: Discrim Score: −0.99
GvH: Signal Score (−7.5): −4.98
Possible site: 28
>>> Seems to have no N-terminal signal seq.
ALOM program count: 0 value: 1.70 threshold: 0.0
PERIPHERAL Likelihood = 1.70 216
modified ALOM score: −0.84
Final Results
bacterial cytoplasm --- Certainty = 0.0695(Affirmative) <succ>
bacterial membrane --- Certainty = 0.0000(Not Clear) <succ>
bacterial outside --- Certainty = 0.0000(Not Clear) <succ>

SEQ ID 64 is predicted to be heat-inducible transcription repressor HrcA (hrcA).

### Example 33

A DNA sequence was identified in S. agalactiae <SEQ ID 65> which encodes the amino acid sequence <SEQ ID 66>. Analysis of this protein sequence reveals the following:

Lipop: Possible site: −1 Crend: 10
McG: Discrim Score: −9.70
GvH: Signal Score (−7.5): −4.45
Possible site: 41
>>> Seems to have no N-terminal signal seq.
ALOM program count: 0 value: 3.07 threshold: 0.0
PERIPHERAL Likelihood = 3.07 388
modified ALOM score: −1.11
Final Results
bacterial cytoplasm --- Certainty = 0.2331(Affirmative) <succ>
bacterial membrane --- Certainty = 0.0000(Not Clear) <succ>
bacterial outside --- Certainty = 0.0000(Not Clear) <succ>

SEQ ID 66 is predicted to be DAK2 domain protein.

### Example 34

A DNA sequence was identified in S. agalactiae <SEQ ID 67> which encodes the amino acid sequence <SEQ ID 68>. Analysis of this protein sequence reveals the following:

Lipop: Possible site: −1 Crend: 7
McG: Discrim Score: −9.07
GvH: Signal Score (−7.5): −4.07
Possible site: 22
>>> Seems to have no N-terminal signal seq.
ALOM program count: 0 value: 2.70 threshold: 0.0
PERIPHERAL Likelihood = 2.70 651
modified ALOM score: −1.04
Final Results
bacterial cytoplasm --- Certainty = 0.2128(Affirmative) <succ>
bacterial membrane --- Certainty = 0.0000(Not Clear) <succ>
bacterial outside --- Certainty = 0.0000(Not Clear) <succ>

SEQ ID 68 is predicted to be DNA-directed RNA polymerase beta′ subunit (rpoC).

### Example 35

A DNA sequence was identified in S. agalactiae <SEQ ID 69> which encodes the amino acid sequence <SEQ ID 70>. Analysis of this protein sequence reveals the following:

Lipop: Possible site: −1 Crend: 8
McG: Discrim Score: −4.26
GvH: Signal Score (−7.5): −5.06
Possible site: 55
>>> Seems to have no N-terminal signal seq.
ALOM program count: 0 value: 6.95 threshold: 0.0
PERIPHERAL Likelihood = 6.95 265
modified ALOM score: −1.89
Final Results
bacterial cytoplasm --- Certainty = 0.1364(Affirmative) <succ>
bacterial membrane --- Certainty = 0.0000(Not Clear) <succ>
bacterial outside --- Certainty = 0.0000(Not Clear) <succ>

SEQ ID 70 is predicted to be glycyl-tRNA synthetase, alpha subunit (glyQ).

### Example 36

A DNA sequence was identified in S. agalactiae <SEQ ID 71> which encodes the amino acid sequence <SEQ ID 72>. Analysis of this protein sequence reveals the following:

Lipop: Possible site: −1 Crend: 6
McG: Discrim Score: 10.31
GvH: Signal Score (−7.5): −5.15
Possible site: 34
>>> Seems to have an uncleavable N-term signal seq
ALOM program count: 2 value: −9.61 threshold: 0.0

| | | | |
|---|---|---|---|
| INTEGRAL | Likelihood = −9.61 | Transmembrane | 50-66 (45-73) |
| INTEGRAL | Likelihood = −8.76 | Transmembrane | 13-29 (7-33) |
| PERIPHERAL | Likelihood = 25.62 | 32 | |

modified ALOM score: 2.42
Final Results
bacterial membrane --- Certainty = 0.4843(Affirmative) <succ>
bacterial outside --- Certainty = 0.0000(Not Clear) <succ>
bacterial cytoplasm --- Certainty = 0.0000(Not Clear) <succ>

SEQ ID 72 is conserved.

### Example 37

A DNA sequence was identified in S. agalactiae <SEQ ID 73> which encodes the amino acid sequence <SEQ ID 74>. Analysis of this protein sequence reveals the following:

Lipop: Possible site: −1 Crend: 10
McG: Discrim Score: −13.72
GvH: Signal Score (−7.5): −5.42
Possible site: 27
>>> Seems to have no N-terminal signal seq.
ALOM program count: 0 value: 3.23 threshold: 0.0
PERIPHERAL Likelihood = 3.23 67
modified ALOM score: −1.15
Final Results
bacterial cytoplasm --- Certainty = 0.3327(Affirmative) <succ>
bacterial membrane --- Certainty = 0.0000(Not Clear) <succ>
bacterial outside --- Certainty = 0.0000(Not Clear) <succ>

SEQ ID 74 is predicted to be DNA-binding response regulator.

### Example 38

A DNA sequence was identified in S. agalactiae <SEQ ID 75> which encodes the amino acid sequence <SEQ ID 76>. Analysis of this protein sequence reveals the following:

Lipop: Possible site: −1 Crend: 10
McG: Discrim Score: −10.31
GvH: Signal Score (−7.5): −3.3
Possible site: 44
>>> Seems to have no N-terminal signal seq.
ALOM program count: 0 value: 2.17 threshold: 0.0
PERIPHERAL Likelihood = 2.17 277
modified ALOM score: −0.93
Final Results
bacterial cytoplasm --- Certainty = 0.2221(Affirmative) <succ>
bacterial membrane --- Certainty = 0.0000(Not Clear) <succ>
bacterial outside --- Certainty = 0.0000(Not Clear) <succ>

SEQ ID 76 is predicted to be transcriptional regulator.

### Example 39

A DNA sequence was identified in S. agalactiae <SEQ ID 77> which encodes the amino acid sequence <SEQ ID 78>. Analysis of this protein sequence reveals the following:

Lipop: Possible site: −1 Crend: 10
McG: Discrim Score: −10.56
GvH: Signal Score (−7.5): −5.38
Possible site: 41
>>> Seems to have no N-terminal signal seq.
ALOM program count: 1 value: −1.22 threshold: 0.0

| | | | |
|---|---|---|---|
| INTEGRAL | Likelihood = −1.22 | Transmembrane | 140-156 |
| | | | (140-156) |
| PERIPHERAL | Likelihood = 3.02 | 46 | |

modified ALOM score: 0.74
Final Results
bacterial membrane --- Certainty = 0.1489(Affirmative) <succ>
bacterial outside --- Certainty = 0.0000(Not Clear) <succ>
bacterial cytoplasm --- Certainty = 0.0000(Not Clear) <succ>

SEQ ID 78 is predicted to be acetyl-CoA carboxylase, carboxyl transferase, alpha subunit (accA).

### Example 40

A DNA sequence was identified in S. agalactiae <SEQ ID 79> which encodes the amino acid sequence <SEQ ID 80>. Analysis of this protein sequence reveals the following:

Lipop: Possible site: −1 Crend: 0
McG: Discrim Score: −17.99
GvH: Signal Score (−7.5): −4.48
Possible site: 28
>>> Seems to have no N-terminal signal seq.
ALOM program count: 0 value: 4.93 threshold: 0.0
PERIPHERAL Likelihood = 4.93 39
modified ALOM score: −1.49
Final Results
bacterial cytoplasm --- Certainty = 0.3995(Affirmative) <succ>
bacterial membrane --- Certainty = 0.0000(Not Clear) <succ>
bacterial outside --- Certainty = 0.0000(Not Clear) <succ>

SEQ ID 80 is conserved.

### Example 41

A DNA sequence was identified in S. agalactiae <SEQ ID 81> which encodes the amino acid sequence <SEQ ID 82>. Analysis of this protein sequence reveals the following:

Lipop: Possible site: −1 Crend: 10
McG: Discrim Score: −12.43
GvH: Signal Score (−7.5): −4.88
Possible site: 53
>>> Seems to have no N-terminal signal seq.
ALOM program count: 0 value: 7.21 threshold: 0.0
PERIPHERAL Likelihood = 7.21 39
modified ALOM score: −1.94
Final Results
bacterial cytoplasm --- Certainty = 0.2962(Affirmative) <succ>
bacterial membrane --- Certainty = 0.0000(Not Clear) <succ>
bacterial outside --- Certainty = 0.0000(Not Clear) <succ>

SEQ ID 82 is predicted to be ribosome-binding factor A (rbfA).

### Example 42

A DNA sequence was identified in S. agalactiae <SEQ ID 83> which encodes the amino acid sequence <SEQ ID 84>. Analysis of this protein sequence reveals the following:

Lipop: Possible site: −1 Crend: 10
McG: Discrim Score: −17.53
GvH: Signal Score (−7.5): −5.73
Possible site: 27
>>> Seems to have no N-terminal signal seq.
ALOM program count: 0 value: 7.96 threshold: 0.0
PERIPHERAL Likelihood = 7.96 116
modified ALOM score: −2.09
Final Results
bacterial cytoplasm --- Certainty = 0.4152(Affirmative) <succ>
bacterial membrane --- Certainty = 0.0000(Not Clear) <succ>
bacterial outside --- Certainty = 0.0000(Not Clear) <succ>

SEQ ID 84 is predicted to be R3H domain protein.

### Example 43

A DNA sequence was identified in S. agalactiae <SEQ ID 85> which encodes the amino acid sequence <SEQ ID 86>. Analysis of this protein sequence reveals the following:

Lipop: Possible site: −1 Crend: 8
McG: Discrim Score: −23.53
GvH: Signal Score (−7.5): −9.34
Possible site: 58
>>> Seems to have no N-terminal signal seq.
ALOM program count: 0 value: 4.88 threshold: 0.0
PERIPHERAL Likelihood = 4.88 113
modified ALOM score: −1.48
Final Results
bacterial cytoplasm --- Certainty = 0.6075(Affirmative) <succ>
bacterial membrane --- Certainty = 0.0000(Not Clear) <succ>
bacterial outside --- Certainty = 0.0000(Not Clear) <succ>

SEQ ID 86 is predicted to be bacteriophage L54a, integrase, truncation.

### Example 44

A DNA sequence was identified in S. agalactiae <SEQ ID 87> which encodes the amino acid sequence <SEQ ID 88>. Analysis of this protein sequence reveals the following:

Lipop: Possible site: −1 Crend: 9
McG: Discrim Score: −15.39
GvH: Signal Score (−7.5): −3.96
Possible site: 54
>>> Seems to have no N-terminal signal seq.
ALOM program count: 0 value: 1.38 threshold: 0.0
PERIPHERAL Likelihood = 1.38 118
modified ALOM score: −0.78
Final Results
bacterial cytoplasm --- Certainty = 0.3370(Affirmative) <succ>
bacterial membrane --- Certainty = 0.0000(Not Clear) <succ>
bacterial outside --- Certainty = 0.0000(Not Clear) <succ>

SEQ ID 88 is predicted to be acetyltransferase, GNAT family.

### Example 45

A DNA sequence was identified in S. agalactiae <SEQ ID 89> which encodes the amino acid sequence <SEQ ID 90>. Analysis of this protein sequence reveals the following:

Lipop: Possible site: −1 Crend: 0
McG: Discrim Score: 0.71
GvH: Signal Score (−7.5): 0.619999
Possible site: 50
>>> Seems to have a cleavable N-term signal seq.
ALOM program count: 0 value: 4.56 threshold: 0.0
PERIPHERAL Likelihood = 4.56 71
modified ALOM score: −1.41
Final Results
bacterial outside --- Certainty = 0.3000(Affirmative) <succ>
bacterial membrane --- Certainty = 0.0000(Not Clear) <succ>
bacterial cytoplasm --- Certainty = 0.0000(Not Clear) <succ>

SEQ ID 90 is conserved.

### Example 46

A DNA sequence was identified in S. agalactiae <SEQ ID 91> which encodes the amino acid sequence <SEQ ID 92>. Analysis of this protein sequence reveals the following:

Lipop: Possible site: −1 Crend: 10
McG: Discrim Score: −12.55
GvH: Signal Score (−7.5): −7.11
Possible site: 19
>>> Seems to have no N-terminal signal seq.
ALOM program count: 0 value: 2.97 threshold: 0.0
PERIPHERAL Likelihood = 2.97 265
modified ALOM score: −1.09
Final Results
bacterial cytoplasm --- Certainty = 0.3433(Affirmative) <succ>
bacterial membrane --- Certainty = 0.0000(Not Clear) <succ>
bacterial outside --- Certainty = 0.0000(Not Clear) <succ>

SEQ ID 92 is conserved.

### Example 47

A DNA sequence was identified in S. agalactiae <SEQ ID 93> which encodes the amino acid sequence <SEQ ID 94>. Analysis of this protein sequence reveals the following:

Lipop: Possible site: −1 Crend: 8
McG: Discrim Score: −12.55
GvH: Signal Score (−7.5): −3.4
Possible site: 44
>>> Seems to have no N-terminal signal seq.
ALOM program count: 0 value: 2.28 threshold: 0.0
PERIPHERAL Likelihood = 2.28 32
modified ALOM score: −0.96
Final Results
bacterial cytoplasm --- Certainty = 0.2691(Affirmative) <succ>
bacterial membrane --- Certainty = 0.0000(Not Clear) <succ>
bacterial outside --- Certainty = 0.0000(Not Clear) <succ>

SEQ ID 94 is predicted to be anthranilate synthase component II (trpG).

### Example 48

A DNA sequence was identified in S. agalactiae <SEQ ID 95> which encodes the amino acid sequence <SEQ ID 96>. Analysis of this protein sequence reveals the following:

Lipop: Possible site: −1 Crend: 8
McG: Discrim Score: −10.19
GvH: Signal Score (−7.5): −5.38
Possible site: 57
>>> Seems to have no N-terminal signal seq.
ALOM program count: 0 value: 1.70 threshold: 0.0
PERIPHERAL Likelihood = 1.70 105
modified ALOM score: −0.84
Final Results
bacterial cytoplasm --- Certainty = 0.2614(Affirmative) <succ>
bacterial membrane --- Certainty = 0.0000(Not Clear) <succ>
bacterial outside --- Certainty = 0.0000(Not Clear) <succ>

SEQ ID 96 is predicted to be geranyltranstransferase.

### Example 49

A DNA sequence was identified in S. agalactiae <SEQ ID 97> which encodes the amino acid sequence <SEQ ID 98>. Analysis of this protein sequence reveals the following:

Lipop: Possible site: −1 Crend: 3
McG: Discrim Score: −18.87
GvH: Signal Score (−7.5): −7.58
Possible site: 42
>>> Seems to have no N-terminal signal seq.
ALOM program count: 0 value: 6.95 threshold: 0.0
PERIPHERAL Likelihood = 6.95 72
modified ALOM score: −1.89
Final Results
bacterial cytoplasm --- Certainty = 0.4789(Affirmative) <succ>
bacterial membrane --- Certainty = 0.0000(Not Clear) <succ>
bacterial outside --- Certainty = 0.0000(Not Clear) <succ>

SEQ ID 98 is conserved.

### Example 50

A DNA sequence was identified in S. agalactiae <SEQ ID 99> which encodes the amino acid sequence <SEQ ID 100>. Analysis of this protein sequence reveals the following:

Lipop: Possible site: −1 Crend: 7
McG: Discrim Score: −14.71
GvH: Signal Score (−7.5): −3.82
Possible site: 50
>>> Seems to have no N-terminal signal seq.
ALOM program count: 0 value: 6.84 threshold: 0.0
PERIPHERAL Likelihood = 6.84 45
modified ALOM score: −1.87
Final Results
bacterial cytoplasm --- Certainty = 0.3205(Affirmative) <succ>
bacterial membrane --- Certainty = 0.0000(Not Clear) <succ>
bacterial outside --- Certainty = 0.0000(Not Clear) <succ>

SEQ ID 100 is conserved.

### Example 51

A DNA sequence was identified in S. agalactiae <SEQ ID 101> which encodes the amino acid sequence <SEQ ID 102>. Analysis of this protein sequence reveals the following:

Lipop: Possible site: −1 Crend: 4
McG: Discrim Score: −4.77
GvH: Signal Score (−7.5): −5.67
Possible site: 42
>>> Seems to have no N-terminal signal seq.
ALOM program count: 0 value: 7.43 threshold: 0.0
PERIPHERAL Likelihood = 7.43 26
modified ALOM score: −1.99
Final Results
bacterial cytoplasm --- Certainty = 0.1588(Affirmative) <succ>
bacterial membrane --- Certainty = 0.0000(Not Clear) <succ>
bacterial outside --- Certainty = 0.0000(Not Clear) <succ>

SEQ ID 102 is conserved.

### Example 52

A DNA sequence was identified in S. agalactiae <SEQ ID 103> which encodes the amino acid sequence <SEQ ID 104>. Analysis of this protein sequence reveals the following:

Lipop: Possible site: −1 Crend: 9
McG: Discrim Score: −30.58
GvH: Signal Score (−7.5): −7.7
Possible site: 34
>>> Seems to have no N-terminal signal seq.
ALOM program count: 0 value: 17.14 threshold: 0.0
PERIPHERAL Likelihood = 17.14 40
modified ALOM score: −3.93
Final Results
bacterial cytoplasm --- Certainty = 0.7157(Affirmative) <succ>
bacterial membrane --- Certainty = 0.0000(Not Clear) <succ>
bacterial outside --- Certainty = 0.0000(Not Clear) <succ>

SEQ ID 104 is conserved.

### Example 53

A DNA sequence was identified in S. agalactiae <SEQ ID 105> which encodes the amino acid sequence <SEQ ID 106>. Analysis of this protein sequence reveals the following:

Lipop: Possible site: −1 Crend: 10
McG: Discrim Score: −13.19
GvH: Signal Score (−7.5): −7
Possible site: 16
>>> Seems to have no N-terminal signal seq.
ALOM program count: 0 value: 7.16 threshold: 0.0
PERIPHERAL Likelihood = 7.16 3
modified ALOM score: −1.93
Final Results
bacterial cytoplasm --- Certainty = 0.3538(Affirmative) <succ>
bacterial membrane --- Certainty = 0.0000(Not Clear) <succ>
bacterial outside --- Certainty = 0.0000(Not Clear) <succ>

### Example 54

A DNA sequence was identified in S. agalactiae <SEQ ID 107> which encodes the amino acid sequence <SEQ ID 108>. Analysis of this protein sequence reveals the following:

Lipop: Possible site: −1 Crend: 9
McG: Discrim Score: −16.03
GvH: Signal Score (−7.5): 0.99
Possible site: 40
>>> Seems to have no N-terminal signal seq.
ALOM program count: 0 value: 12.89 threshold: 0.0
PERIPHERAL Likelihood = 12.89 71
modified ALOM score: −3.08
Final Results
bacterial cytoplasm --- Certainty = 0.2509(Affirmative) <succ>
bacterial membrane --- Certainty = 0.0000(Not Clear) <succ>
bacterial outside --- Certainty = 0.0000(Not Clear) <succ>

SEQ ID 108 is predicted to be transposase OrfA, IS3 family.

### Example 55

A DNA sequence was identified in S. agalactiae <SEQ ID 109> which encodes the amino acid sequence <SEQ ID 110>. Analysis of this protein sequence reveals the following:

Lipop: Possible site: −1 Crend: 10
McG: Discrim Score: −17.01
GvH: Signal Score (−7.5): −3.03
Possible site: 51
>>> Seems to have no N-terminal signal seq.
ALOM program count: 0 value: 1.96 threshold: 0.0
PERIPHERAL Likelihood = 1.96 148
modified ALOM score: −0.89
Final Results
bacterial cytoplasm --- Certainty = 0.3508(Affirmative) <succ>
bacterial membrane --- Certainty = 0.0000(Not Clear) <succ>
bacterial outside --- Certainty = 0.0000(Not Clear) <succ>

SEQ ID 110 is predicted to be D-lactate dehydrogenase (IdhA).

### Example 56

A DNA sequence was identified in S. agalactiae <SEQ ID 111> which encodes the amino acid sequence <SEQ ID 112>. Analysis of this protein sequence reveals the following:

Lipop: Possible site: −1 Crend: 10
McG: Discrim Score: −11.46
GvH: Signal Score (−7.5): −1.07
Possible site: 40
>>> Seems to have no N-terminal signal seq.
ALOM program count: 0 value: 1.27 threshold: 0.0
PERIPHERAL Likelihood = 1.27 123
modified ALOM score: −0.75
Final Results
bacterial cytoplasm --- Certainty = 0.2007(Affirmative) <succ>
bacterial membrane --- Certainty = 0.0000(Not Clear) <succ>
bacterial outside --- Certainty = 0.0000(Not Clear) <succ>

SEQ ID 112 is predicted to be ribonuclease III (rnc).

### Example 57

A DNA sequence was identified in S. agalactiae <SEQ ID 113> which encodes the amino acid sequence <SEQ ID 114>. Analysis of this protein sequence reveals the following:

Lipop: Possible site: −1 Crend: 1
McG: Discrim Score: −5.90
GvH: Signal Score (−7.5): −8.56
Possible site: 19
>>> Seems to have no N-terminal signal seq.
ALOM program count: 0 value: 3.29 threshold: 0.0
PERIPHERAL Likelihood = 3.29 688
modified ALOM score: −1.16
Final Results
bacterial cytoplasm --- Certainty = 0.2393(Affirmative) <succ>
bacterial membrane --- Certainty = 0.0000(Not Clear) <succ>
bacterial outside --- Certainty = 0.0000(Not Clear) <succ>

SEQ ID 114 is predicted to be transcriptional accessory protein Tex.

### Example 58

A DNA sequence was identified in S. agalactiae <SEQ ID 115> which encodes the amino acid sequence <SEQ ID 116>. Analysis of this protein sequence reveals the following:

Lipop: Possible site: −1 Crend: 10
McG: Discrim Score: −13.95
GvH: Signal Score (−7.5): −6.29
Possible site: 20
>>> Seems to have no N-terminal signal seq.
ALOM program count: 0 value: 12.57 threshold: 0.0
PERIPHERAL Likelihood = 12.57 5
modified ALOM score: −3.01
Final Results
bacterial cytoplasm --- Certainty = 0.3549(Affirmative) <succ>
bacterial membrane --- Certainty = 0.0000(Not Clear) <succ>
bacterial outside --- Certainty = 0.0000(Not Clear) <succ>

SEQ ID 116 is conserved.

### Example 59

A DNA sequence was identified in S. agalactiae <SEQ ID 117> which encodes the amino acid sequence <SEQ ID 118>. Analysis of this protein sequence reveals the following:

Lipop: Possible site: −1 Crend: 10
McG: Discrim Score: −22.65
GvH: Signal Score (−7.5): −4.43
Possible site: 55
>>> Seems to have no N-terminal signal seq.
ALOM program count: 0 value: 6.26 threshold: 0.0
PERIPHERAL Likelihood = 6.26 29
modified ALOM score: −1.75
Final Results
bacterial cytoplasm --- Certainty = 0.4916(Affirmative) <succ>
bacterial membrane --- Certainty = 0.0000(Not Clear) <succ>
bacterial outside --- Certainty = 0.0000(Not Clear) <succ>

SEQ ID 118 is predicted to be conserved domain protein.

### Example 60

A DNA sequence was identified in S. agalactiae <SEQ ID 119> which encodes the amino acid sequence <SEQ ID 120>. Analysis of this protein sequence reveals the following:

Lipop: Possible site: −1 Crend: 8
McG: Discrim Score: −14.27
GvH: Signal Score (−7.5): −6.02
Possible site: 49
>>> Seems to have no N-terminal signal seq.
ALOM program count: 0 value: 4.03 threshold: 0.0
PERIPHERAL Likelihood = 4.03 56
modified ALOM score: −1.31
Final Results
bacterial cytoplasm --- Certainty = 0.3559(Affirmative) <succ>
bacterial membrane --- Certainty = 0.0000(Not Clear) <succ>
bacterial outside --- Certainty = 0.0000(Not Clear) <succ>

SEQ ID 120 is predicted to be acetyltransferase, GNAT family.

### Example 61

A DNA sequence was identified in S. agalactiae <SEQ ID 121> which encodes the amino acid sequence <SEQ ID 122>. Analysis of this protein sequence reveals the following:

Lipop: Possible site: −1 Crend: 8
McG: Discrim Score: −8.15
GvH: Signal Score (−7.5): −5.83
Possible site: 61
>>> Seems to have no N-terminal signal seq.
ALOM program count: 0 value: 3.18 threshold: 0.0
PERIPHERAL Likelihood = 3.18 230
modified ALOM score: −1.14
Final Results
bacterial cytoplasm --- Certainty = 0.2296(Affirmative) <succ>
bacterial membrane --- Certainty = 0.0000(Not Clear) <succ>
bacterial outside --- Certainty = 0.0000(Not Clear) <succ>

SEQ ID 122 is predicted to be nucleoside diphosphate kinase domain protein.

### Example 62

A DNA sequence was identified in S. agalactiae <SEQ ID 123> which encodes the amino acid sequence <SEQ ID 124>. Analysis of this protein sequence reveals the following:

Lipop: Possible site: −1 Crend: 10
McG: Discrim Score: −20.24
GvH: Signal Score (−7.5): −6.61
Possible site: 61
>>> Seems to have no N-terminal signal seq.
ALOM program count: 0 value: 6.47 threshold: 0.0
PERIPHERAL Likelihood = 6.47 111
modified ALOM score: −1.79
Final Results
bacterial cytoplasm --- Certainty = 0.4870(Affirmative) <succ>
bacterial membrane --- Certainty = 0.0000(Not Clear) <succ>
bacterial outside --- Certainty = 0.0000(Not Clear) <succ>

SEQ ID 124 is predicted to be Tn916.

### Example 63

A DNA sequence was identified in S. agalactiae <SEQ ID 125> which encodes the amino acid sequence <SEQ ID 126>. Analysis of this protein sequence reveals the following:

Lipop: Possible site: −1 Crend: 7
McG: Discrim Score: −14.87
GvH: Signal Score (−7.5): −4.02
Possible site: 28
>>> Seems to have no N-terminal signal seq.
ALOM program count: 0 value: 8.91 threshold: 0.0
PERIPHERAL Likelihood = 8.91 112
modified ALOM score: −2.28
Final Results
bacterial cytoplasm --- Certainty = 0.3279(Affirmative) <succ>
bacterial membrane --- Certainty = 0.0000(Not Clear) <succ>
bacterial outside --- Certainty = 0.0000(Not Clear) <succ>

SEQ ID 126 is predicted to be cytidine deaminase (cdd).

### Example 64

A DNA sequence was identified in S. agalactiae <SEQ ID 127> which encodes the amino acid sequence <SEQ ID 128>. Analysis of this protein sequence reveals the following:

Lipop: Possible site: −1 Crend: 10
McG: Discrim Score: −8.61
GvH: Signal Score (−7.5): −7.88
Possible site: 32
>>> Seems to have no N-terminal signal seq.
ALOM program count: 0 value: 14.75 threshold: 0.0
PERIPHERAL Likelihood = 14.75 76
modified ALOM score: −3.45
Final Results
bacterial cytoplasm --- Certainty = 0.2798(Affirmative) <succ>
bacterial membrane --- Certainty = 0.0000(Not Clear) <succ>
bacterial outside --- Certainty = 0.0000(Not Clear) <succ>

SEQ ID 128 is conserved.

### Example 65

A DNA sequence was identified in S. agalactiae <SEQ ID 129> which encodes the amino acid sequence <SEQ ID 130>. Analysis of this protein sequence reveals the following:

Lipop: Possible site: −1 Crend: 10
McG: Discrim Score: −12.49
GvH: Signal Score (−7.5): −9.71
Possible site: 22
>>> Seems to have no N-terminal signal seq.
ALOM program count: 0 value: 0.26 threshold: 0.0
PERIPHERAL Likelihood = 0.26 76
modified ALOM score: −0.55
Final Results
bacterial cytoplasm --- Certainty = 0.3940(Affirmative) <succ>
bacterial membrane --- Certainty = 0.0000(Not Clear) <succ>
bacterial outside --- Certainty = 0.0000(Not Clear) <succ>

SEQ ID 130 is predicted to be dihydroorotase, multifunctional complex type (pyrC).

### Example 66

A DNA sequence was identified in S. agalactiae <SEQ ID 131> which encodes the amino acid sequence <SEQ ID 132>. Analysis of this protein sequence reveals the following:

Lipop: Possible site: −1 Crend: 1
McG: Discrim Score: −6.23
GvH: Signal Score (−7.5): −7.98
Possible site: 29
>>> Seems to have no N-terminal signal seq.
ALOM program count: 0 value: 6.10 threshold: 0.0
PERIPHERAL Likelihood = 6.10 84
modified ALOM score: −1.72
Final Results
bacterial cytoplasm --- Certainty = 0.2341(Affirmative) <succ>
bacterial membrane --- Certainty = 0.0000(Not Clear) <succ>
bacterial outside --- Certainty = 0.0000(Not Clear) <succ>

SEQ ID 132 is predicted to be flavoprotein-related protein.

### Example 67

A DNA sequence was identified in S. agalactiae <SEQ ID 133> which encodes the amino acid sequence <SEQ ID 134>. Analysis of this protein sequence reveals the following:

Lipop: Possible site: −1 Crend: 4
McG: Discrim Score: −6.04
GvH: Signal Score (−7.5): −6.37
Possible site: 15
>>> Seems to have no N-terminal signal seq.
ALOM program count: 0 value: 3.76 threshold: 0.0
PERIPHERAL Likelihood = 3.76 308
modified ALOM score: −1.25
Final Results
bacterial cytoplasm --- Certainty = 0.1981(Affirmative) <succ>
bacterial membrane --- Certainty = 0.0000(Not Clear) <succ>
bacterial outside --- Certainty = 0.0000(Not Clear) <succ>

SEQ ID 134 is predicted to be cysteine desulphurase (iscS).

### Example 68

A DNA sequence was identified in S. agalactiae <SEQ ID 135> which encodes the amino acid sequence <SEQ ID 136>. Analysis of this protein sequence reveals the following:

Lipop: Possible site: −1 Crend: 10
McG: Discrim Score: −10.75
GvH: Signal Score (−7.5): −5.76
Possible site: 41
>>> Seems to have no N-terminal signal seq.
ALOM program count: 0 value: 2.49 threshold: 0.0
PERIPHERAL Likelihood = 2.49 162
modified ALOM score: −1.00
Final Results
bacterial cytoplasm --- Certainty = 0.2802(Affirmative) <succ>
bacterial membrane --- Certainty = 0.0000(Not Clear) <succ>
bacterial outside --- Certainty = 0.0000(Not Clear) <succ>

SEQ ID 136 is predicted to be DNA-binding protein.

### Example 69

A DNA sequence was identified in S. agalactiae <SEQ ID 137> which encodes the amino acid sequence <SEQ ID 138>. Analysis of this protein sequence reveals the following:

Lipop: Possible site: −1 Crend: 5
McG: Discrim Score: −11.91
GvH: Signal Score (−7.5): −7.15
Possible site: 26
>>> Seems to have no N-terminal signal seq.
ALOM program count: 0 value: 1.75 threshold: 0.0
PERIPHERAL Likelihood = 1.75 292
modified ALOM score: −0.85
Final Results
bacterial cytoplasm --- Certainty = 0.3312(Affirmative) <succ>
bacterial membrane --- Certainty = 0.0000(Not Clear) <succ>
bacterial outside --- Certainty = 0.0000(Not Clear) <succ>

SEQ ID 138 is predicted to be ribosomal protein S1 (rpsA).

### Example 70

A DNA sequence was identified in S. agalactiae <SEQ ID 139> which encodes the amino acid sequence <SEQ ID 140>. Analysis of this protein sequence reveals the following:

Lipop: Possible site: −1 Crend: 9
McG: Discrim Score: −0.79
GvH: Signal Score (−7.5): −2.01
Possible site: 58
>>> Seems to have no N-terminal signal seq.
ALOM program count: 0 value: 4.29 threshold: 0.0
PERIPHERAL Likelihood = 4.29 16
modified ALOM score: −1.36
Final Results
bacterial cytoplasm --- Certainty = 0.0060(Affirmative) <succ>
bacterial membrane --- Certainty = 0.0000(Not Clear) <succ>
bacterial outside --- Certainty = 0.0000(Not Clear) <succ>

SEQ ID 140 is predicted to be MutT/nudix family protein.

### Example 71

A DNA sequence was identified in S. agalactiae <SEQ ID 141> which encodes the amino acid sequence <SEQ ID 142>. Analysis of this protein sequence reveals the following:

Lipop: Possible site: −1 Crend: 4
McG: Discrim Score: −11.70
GvH: Signal Score (−7.5): −2.55
Possible site: 29
>>> Seems to have no N-terminal signal seq.
ALOM program count: 0 value: 5.83 threshold: 0.0
PERIPHERAL Likelihood = 5.83 69
modified ALOM score: −1.67
Final Results
bacterial cytoplasm --- Certainty = 0.2350(Affirmative) <succ>
bacterial membrane --- Certainty = 0.0000(Not Clear) <succ>
bacterial outside --- Certainty = 0.0000(Not Clear) <succ>

SEQ ID 142 is conserved.

### Example 72

A DNA sequence was identified in S. agalactiae <SEQ ID 143> which encodes the amino acid sequence <SEQ ID 144>. Analysis of this protein sequence reveals the following:

Lipop: Possible site: −1 Crend: 9
McG: Discrim Score: −16.03
GvH: Signal Score (−7.5): 0.44
Possible site: 40
>>> Seems to have no N-terminal signal seq.
ALOM program count: 0 value: 14.16 threshold: 0.0
PERIPHERAL Likelihood = 14.16 27
modified ALOM score: −3.33
Final Results
bacterial cytoplasm --- Certainty = 0.2619(Affirmative) <succ>
bacterial membrane --- Certainty = 0.0000(Not Clear) <succ>
bacterial outside --- Certainty = 0.0000(Not Clear) <succ>

SEQ ID 144 is predicted to be transposase OrfA, IS3 family.

### Example 73

A DNA sequence was identified in S. agalactiae <SEQ ID 145> which encodes the amino acid sequence <SEQ ID 146>. Analysis of this protein sequence reveals the following:

Lipop: Possible site: −1 Crend: 8
McG: Discrim Score: −13.64
GvH: Signal Score (−7.5): −1.95
Possible site: 16
>>> Seems to have no N-terminal signal seq.
ALOM program count: 0 value: 2.38 threshold: 0.0
PERIPHERAL Likelihood = 2.38 124
modified ALOM score: −0.98
Final Results
bacterial cytoplasm - - - Certainty = 0.2618(Affirmative) <succ>
bacterial membrane - - - Certainty = 0.0000(Not Clear) <succ>
bacterial outside - - - Certainty = 0.0000(Not Clear) <succ>

SEQ ID 146 is predicted to be ISSdy1, transposase OrfB.

### Example 74

A DNA sequence was identified in S. agalactiae <SEQ ID 147> which encodes the amino acid sequence <SEQ ID 148>. Analysis of this protein sequence reveals the following:

Lipop: Possible site: −1 Crend: 6
McG: Discrim Score: −11.45
GvH: Signal Score (−7.5): −8.98
Possible site: 47
>>> Seems to have no N-terminal signal seq.
ALOM program count: 0 value: 10.13 threshold: 0.0
PERIPHERAL Likelihood = 10.13 2
modified ALOM score: −2.53
Final Results
bacterial cytoplasm - - - Certainty = 0.3585(Affirmative) <succ>
bacterial membrane - - - Certainty = 0.0000(NotClear) <succ>
bacterial outside - - - Certainty = 0.0000(NotClear) <succ>

SEQ ID 148 is predicted to be IS861, transposase OrfB, truncation.

### Example 75

A DNA sequence was identified in S. agalactiae <SEQ ID 149> which encodes the amino acid sequence <SEQ ID 150>. Analysis of this protein sequence reveals the following:

Lipop: Possible site: −1 Crend: 9
McG: Discrim Score: −14.51
GvH: Signal Score (−7.5): −8.11
Possible site: 47
>>> Seems to have no N-terminal signal seq.
ALOM program count: 0 value: 11.62 threshold: 0.0
PERIPHERAL Likelihood = 11.62 44
modified ALOM score: −2.82
Final Results
bacterial cytoplasm - - - Certainty = 0.4025(Affirmative) <succ>
bacterial membrane - - - Certainty = 0.0000(Not Clear) <succ>
bacterial outside - - - Certainty = 0.0000(Not Clear) <succ>

SEQ ID 150 is conserved.

### Example 76

A DNA sequence was identified in S. agalactiae <SEQ ID 151> which encodes the amino acid sequence <SEQ ID 152>. Analysis of this protein sequence reveals the following:

Lipop: Possible site: −1 Crend: 10
McG: Discrim Score: 1.37
GvH: Signal Score (−7.5): −3.76
Possible site: 24
>>> Seems to have an uncleavable N-term signal seq
ALOM program count: 0 value: 2.01 threshold: 0.0
PERIPHERAL Likelihood = 2.01 13
modified ALOM score: −0.90
Final Results
bacterial membrane - - - Certainty = 0.0000(Not Clear) <succ>
bacterial outside - - - Certainty = 0.0000(Not Clear) <succ>
bacterial cytoplasm - - - Certainty = 0.0000(Not Clear) <succ>

SEQ ID 152 is predicted to be ribosomal protein L10 (rp1J).

### Example 77

A DNA sequence was identified in S. agalactiae <SEQ ID 153> which encodes the amino acid sequence <SEQ ID 154>. Analysis of this protein sequence reveals the following:

Lipop: Possible site: −1 Crend: 6
McG: Discrim Score: −7.41
GvH: Signal Score (−7.5): −0.33
Possible site: 58
>>> Seems to have no N-terminal signal seq.
ALOM program count: 0 value: 3.55 threshold: 0.0
PERIPHERAL Likelihood = 3.55 274
modified ALOM score: −1.21
Final Results
bacterial cytoplasm - - - Certainty = 0.1048(Affirmative) <succ>
bacterial membrane - - - Certainty = 0.0000(Not Clear) <succ>
bacterial outside - - - Certainty = 0.0000(Not Clear) <succ>

SEQ ID 154 is predicted to be HMG-CoA synthase.

### Example 78

A DNA sequence was identified in S. agalactiae <SEQ ID 155> which encodes the amino acid sequence <SEQ ID 156>. Analysis of this protein sequence reveals the following:

Lipop: Possible site: −1 Crend: 7
McG: Discrim Score: −11.18
GvH: Signal Score (−7.5): −6.54
Possible site: 17
>>> Seems to have no N-terminal signal seq.
ALOM program count: 0 value: 4.35 threshold: 0.0
PERIPHERAL Likelihood = 4.35 140
modified ALOM score: −1.37
Final Results
bacterial cytoplasm - - - Certainty = 0.3043(Affirmative) <succ>
bacterial membrane - - - Certainty = 0.0000(Not Clear) <succ>
bacterial outside - - - Certainty = 0.0000(Not Clear) <succ>

SEQ ID 156 is predicted to be 16S rRNA processing protein RimM (rimM).

### Example 79

A DNA sequence was identified in S. agalactiae <SEQ ID 157> which encodes the amino acid sequence <SEQ ID 158>. Analysis of this protein sequence reveals the following:

Lipop: Possible site: −1 Crend: 10
McG: Discrim Score: −24.00
GvH: Signal Score (−7.5): −2.33
Possible site: 33
>>> Seems to have no N-terminal signal seq.
ALOM program count: 0 value: 10.61 threshold: 0.0
PERIPHERAL Likelihood = 10.61 29
modified ALOM score: −2.62
Final Results
bacterial cytoplasm - - - Certainty = 0.4767(Affirmative) <succ>
bacterial membrane - - - Certainty = 0.0000(Not Clear) <succ>
bacterial outside - - - Certainty = 0.0000(Not Clear) <succ>

SEQ ID 158 is predicted to be ribosomal protein L27 (rpmA).

### Example 80

A DNA sequence was identified in S. agalactiae <SEQ ID 159> which encodes the amino acid sequence <SEQ ID 160>. Analysis of this protein sequence reveals the following:

Lipop: Possible site: −1 Crend: 8
McG: Discrim Score: −20.57
GvH: Signal Score (−7.5): −5.49
Possible site: 45
>>> Seems to have no N-terminal signal seq.
ALOM program count: 0 value: 2.92 threshold: 0.0
PERIPHERAL Likelihood = 2.92 371
modified ALOM score: −1.08
Final Results
bacterial cytoplasm - - - Certainty = 0.4711(Affirmative) <succ>
bacterial membrane - - - Certainty = 0.0000(Not Clear) <succ>
bacterial outside - - - Certainty = 0.0000(Not Clear) <succ>

SEQ ID 160 is predicted to be thiamine biosynthesis protein ThiI (thiI).

### Example 81

A DNA sequence was identified in S. agalactiae <SEQ ID 161> which encodes the amino acid sequence <SEQ ID 162>. Analysis of this protein sequence reveals the following:

Lipop: Possible site: −1 Crend: 4
McG: Discrim Score: −8.37
GvH: Signal Score (−7.5): −4.96
Possible site: 43
>>> Seems to have no N-terminal signal seq.
ALOM program count: 0 value: 6.52 threshold: 0.0
PERIPHERAL Likelihood = 6.52 36
modified ALOM score: −1.80
Final Results
bacterial cytoplasm - - - Certainty = 0.2166(Affirmative) <succ>
bacterial membrane - - - Certainty = 0.0000(Not Clear) <succ>
bacterial outside - - - Certainty = 0.0000(Not Clear) <succ>

SEQ ID 162 is predicted to be RNA polymerase sigma-70 factor (rpoD).

### Example 82

A DNA sequence was identified in S. agalactiae <SEQ ID 163> which encodes the amino acid sequence <SEQ ID 164>. Analysis of this protein sequence reveals the following:

Lipop: Possible site: −1 Crend: 8
McG: Discrim Score: −12.31
GvH: Signal Score (−7.5): −7.51
Possible site: 13
>>> Seems to have no N-terminal signal seq.
ALOM program count: 0 value: 4.29 threshold: 0.0
PERIPHERAL Likelihood = 4.29 20
modified ALOM score: −1.36
Final Results
bacterial cytoplasm - - - Certainty = 0.3464(Affirmative) <succ>
bacterial membrane - - - Certainty = 0.0000(Not Clear) <succ>
bacterial outside - - - Certainty = 0.0000(Not Clear) <succ>

SEQ ID 164 is predicted to be DNA primase (dnaG).

### Example 83

A DNA sequence was identified in S. agalactiae <SEQ ID 165> which encodes the amino acid sequence <SEQ ID 166>. Analysis of this protein sequence reveals the following:

Lipop: Possible site: −1 Crend: 9
McG: Discrim Score: −19.58
GvH: Signal Score (−7.5): −6.45
Possible site: 26
>>> Seems to have no N-terminal signal seq.
ALOM program count: 0 value: 21.01 threshold: 0.0
PERIPHERAL Likelihood = 21.01 13
modified ALOM score: −4.70
Final Results
bacterial cytoplasm - - - Certainty = 0.4706(Affirmative) <succ>
bacterial membrane - - - Certainty = 0.0000(Not Clear) <succ>
bacterial outside - - - Certainty = 0.0000(Not Clear) <succ>

SEQ ID 166 is predicted to be ribosomal protein S21 (rpsU).

### Example 84

A DNA sequence was identified in S. agalactiae <SEQ ID 167> which encodes the amino acid sequence <SEQ ID 168>. Analysis of this protein sequence reveals the following:

Lipop: Possible site: −1 Crend: 0
McG: Discrim Score: 13.58
GvH: Signal Score (−7.5): −5.58
Possible site: 13
>>> Seems to have an uncleavable N-term signal seq
ALOM program count: 2 value: −14.65 threshold: 0.0

| | | | |
|---|---|---|---|
| INTEGRAL | Likelihood = −14.65 | Transmembrane | 4-20 (1-26) |
| INTEGRAL | Likelihood = −10.93 | Transmembrane | 59-75 (52-77) |
| PERIPHERAL | Likelihood = 17.40 | 38 | |

modified ALOM score: 3.43
Final Results
bacterial membrane - - - Certainty = 0.6859(Affirmative) <succ>
bacterial outside - - - Certainty = 0.0000(Not Clear) <succ>
bacterial cytoplasm - - - Certainty = 0.0000(Not Clear) <succ>

SEQ ID 168 is predicted to be preprotein translocase, SecG subunit.

### Example 85

A DNA sequence was identified in S. agalactiae <SEQ ID 169> which encodes the amino acid sequence <SEQ ID 170>. Analysis of this protein sequence reveals the following:

Lipop: Possible site: −1 Crend: 1
McG: Discrim Score: 9.62
GvH: Signal Score (−7.5): −4.03
Possible site: 53
>>> Seems to have an uncleavable N-term signal seq
ALOM program count: 3 value: −4.09 threshold: 0.0

| | | | |
|---|---|---|---|
| INTEGRAL | Likelihood = −4.09 | Transmembrane | 29-45 (28-48) |
| INTEGRAL | Likelihood = −0.85 | Transmembrane | 8-24 (7-24) |
| INTEGRAL | Likelihood = −0.32 | Transmembrane | 62-78 (62-78) |
| PERIPHERAL | Likelihood = 26.74 | 45 | |

modified ALOM score: 1.32
Final Results
bacterial membrane - - - Certainty = 0.2635(Affirmative) <succ>
bacterial outside - - - Certainty = 0.0000(Not Clear) <succ>
bacterial cytoplasm - - - Certainty = 0.0000(Not Clear) <succ>

SEQ ID 170 is predicted to be a protease.

### Example 86

A DNA sequence was identified in S. agalactiae <SEQ ID 171> which encodes the amino acid sequence <SEQ ID 172>. Analysis of this protein sequence reveals the following:

Lipop: Possible site: −1 Crend: 7
McG: Discrim Score: −9.30
GvH: Signal Score (−7.5): −3.84
Possible site: 56
>>> Seems to have no N-terminal signal seq.
ALOM program count: 1 value: −0.32 threshold: 0.0

| | | | |
|---|---|---|---|
| INTEGRAL | Likelihood = −0.32 | Transmembrane | 157-173 |
| | | | (157-173) |
| PERIPHERAL | Likelihood = 5.04 | 43 | |

modified ALOM score: 0.56
Final Results
bacterial membrane - - - Certainty = 0.1128(Affirmative) <succ>
bacterial outside - - - Certainty = 0.0000(Not Clear) <succ>
bacterial cytoplasm - - - Certainty = 0.0000(Not Clear) <succ>

SEQ ID 172 is predicted to be peptide ABC transporter, ATP-binding protein.

### Example 87

A DNA sequence was identified in S. agalactiae <SEQ ID 173> which encodes the amino acid sequence <SEQ ID 174>. Analysis of this protein sequence reveals the following:

Lipop: Possible site: −1 Crend: 10
McG: Discrim Score: −6.90
GvH: Signal Score (−7.5): 0.25
Possible site: 36
>>> Seems to have no N-terminal signal seq.
ALOM program count: 0 value: 1.38 threshold: 0.0
PERIPHERAL Likelihood = 1.38 71
modified ALOM score: −0.78
Final Results
bacterial cytoplasm - - - Certainty = 0.0830(Affirmative) <succ>
bacterial membrane - - - Certainty = 0.0000(Not Clear) <succ>
bacterial outside - - - Certainty = 0.0000(Not Clear) <succ>

SEQ ID 174 is conserved.

### Example 88

A DNA sequence was identified in S. agalactiae <SEQ ID 175> which encodes the amino acid sequence <SEQ ID 176>. Analysis of this protein sequence reveals the following:

Lipop: Possible site: −1 Crend: 3
McG: Discrim Score: 24.24
GvH: Signal Score (−7.5): −1.68
Possible site: 41
>>> Seems to have a cleavable N-term signal seq.
ALOM program count: 10 value: −9.66 threshold: 0.0

| | | | |
|---|---|---|---|
| INTEGRAL | Likelihood = −9.66 | Transmembrane | 359-375 |
| | | | (355-378) |
| INTEGRAL | Likelihood = −8.01 | Transmembrane | 156-172 |
| | | | (145-175) |
| INTEGRAL | Likelihood = −6.64 | Transmembrane | 434-450 |
| | | | (432-453) |
| INTEGRAL | Likelihood = −5.79 | Transmembrane | 81-97 |
| | | | (78-98) |
| INTEGRAL | Likelihood = −5.20 | Transmembrane | 238-254 |
| | | | (231-258) |
| INTEGRAL | Likelihood = −4.14 | Transmembrane | 45-61 |
| | | | (42-63) |
| INTEGRAL | Likelihood = −3.98 | Transmembrane | 291-307 |
| | | | (288-309) |
| INTEGRAL | Likelihood = −3.72 | Transmembrane | 118-134 |
| | | | (117-137) |
| INTEGRAL | Likelihood = −2.28 | Transmembrane | 199-215 |
| | | | (199-218) |
| INTEGRAL | Likelihood = −0.80 | Transmembrane | 331-347 |
| | | | (331-347) |
| PERIPHERAL | Likelihood = 3.02 | 411 | |

modified ALOM score: 2.43
Final Results
bacterial membrane --- Certainty = 0.4864(Affirmative) <succ>
bacterial outside --- Certainty = 0.0000(Not Clear) <succ>
bacterial cytoplasm --- Certainty = 0.0000(Not Clear) <succ>

SEQ ID 176 is predicted to be branched-chain amino acid transport system II carrier protein (brnQ).

### Example 89

A DNA sequence was identified in S. agalactiae <SEQ ID 177> which encodes the amino acid sequence <SEQ ID 178>. Analysis of this protein sequence reveals the following:

Lipop: Possible site: −1 Crend: 10
McG: Discrim Score: −16.49
GvH: Signal Score (−7.5): −0.0300007
Possible site: 47
>>> Seems to have no N-terminal signal seq.
ALOM program count: 0 value: 3.39 threshold: 0.0
PERIPHERAL Likelihood = 3.39 222
modified ALOM score: −1.18
Final Results
bacterial cytoplasm --- Certainty = 0.2803(Affirmative) <succ>
bacterial membrane --- Certainty = 0.0000(Not Clear) <succ>
bacterial outside --- Certainty = 0.0000(Not Clear) <succ>

SEQ ID 178 is predicted to be aminotransferase, class I.

### Example 90

A DNA sequence was identified in S. agalactiae <SEQ ID 179> which encodes the amino acid sequence <SEQ ID 180>. Analysis of this protein sequence reveals the following:

Lipop: Possible site: −1 Crend: 10
McG: Discrim Score: −11.93
GvH: Signal Score (−7.5): −0.770001
Possible site: 42
>>> Seems to have no N-terminal signal seq.
ALOM program count: 0 value: 3.34 threshold: 0.0
PERIPHERAL Likelihood = 3.34 788
modified ALOM score: −1.17
Final Results
bacterial cytoplasm --- Certainty = 0.2040(Affirmative) <succ>
bacterial membrane --- Certainty = 0.0000(Not Clear) <succ>
bacterial outside --- Certainty = 0.0000(Not Clear) <succ>

SEQ ID 180 is predicted to be excinuclease ABC, A subunit (uvrA).

### Example 91

A DNA sequence was identified in S. agalactiae <SEQ ID 181> which encodes the amino acid sequence <SEQ ID 182>. Analysis of this protein sequence reveals the following:

Lipop: Possible site: −1 Crend: 9
McG: Discrim Score: −10.92
GvH: Signal Score (−7.5): −7.85
Possible site: 40
>>> Seems to have no N-terminal signal seq.
ALOM program count: 0 value: 5.04 threshold: 0.0
PERIPHERAL Likelihood = 5.04 340
modified ALOM score: −1.51
Final Results
bacterial cytoplasm --- Certainty = 0.3254(Affirmative) <succ>
bacterial membrane --- Certainty = 0.0000(Not Clear) <succ>
bacterial outside --- Certainty = 0.0000(Not Clear) <succ>

SEQ ID 182 is predicted to be MutS2 family protein.

### Example 92

A DNA sequence was identified in S. agalactiae <SEQ ID 183> which encodes the amino acid sequence <SEQ ID 184>. Analysis of this protein sequence reveals the following:

Lipop: Possible site: −1 Crend: 10
McG: Discrim Score: −20.42
GvH: Signal Score (−7.5): −3.77
Possible site: 35
>>> Seems to have no N-terminal signal seq.
ALOM program count: 0 value: 0.69 threshold: 0.0
PERIPHERAL Likelihood = 0.69 164
modified ALOM score: −0.64
Final Results
bacterial cytoplasm --- Certainty = 0.4338(Affirmative) <succ>
bacterial membrane --- Certainty = 0.0000(Not Clear) <succ>
bacterial outside --- Certainty = 0.0000(Not Clear) <succ>

SEQ ID 184 is predicted to be ribonuclease Hill (rnhC).

### Example 93

A DNA sequence was identified in S. agalactiae <SEQ ID 185> which encodes the amino acid sequence <SEQ ID 186>. Analysis of this protein sequence reveals the following:

Lipop: Possible site: −1 Crend: 8
McG: Discrim Score: −12.18
GvH: Signal Score (−7.5): −6.61
Possible site: 26
>>> Seems to have no N-terminal signal seq.
ALOM program count: 0 value: 0.95 threshold: 0.0
PERIPHERAL Likelihood = 0.95 56
modified ALOM score: −0.69
Final Results
bacterial cytoplasm --- Certainty = 0.3257(Affirmative) <succ>
bacterial membrane --- Certainty = 0.0000(Not Clear) <succ>
bacterial outside --- Certainty = 0.0000(Not Clear) <succ>

SEQ ID 186 is conserved.

### Example 94

A DNA sequence was identified in S. agalactiae <SEQ ID 187> which encodes the amino acid sequence <SEQ ID 188>. Analysis of this protein sequence reveals the following:

Lipop: Possible site: −1 Crend: 10
McG: Discrim Score: −8.34
GvH: Signal Score (−7.5): −3.13
Possible site: 46
>>> Seems to have no N-terminal signal seq.
ALOM program count: 0 value: 5.36 threshold: 0.0
PERIPHERAL Likelihood = 5.36 128
modified ALOM score: −1.57
Final Results
bacterial cytoplasm --- Certainty = 0.1795(Affirmative) <succ>
bacterial membrane --- Certainty = 0.0000(Not Clear) <succ>
bacterial outside --- Certainty = 0.0000(Not Clear) <succ>

SEQ ID 188 is predicted to be exonuclease.

### Example 95

A DNA sequence was identified in S. agalactiae <SEQ ID 189> which encodes the amino acid sequence <SEQ ID 190>. Analysis of this protein sequence reveals the following:

Lipop: Possible site: −1 Crend: 7
McG: Discrim Score: −11.92
GvH: Signal Score (−7.5): −5.74
Possible site: 58
>>> Seems to have no N-terminal signal seq.
ALOM program count: 0 value: 7.69 threshold: 0.0
PERIPHERAL Likelihood = 7.691 67
modified ALOM score: −2.04
Final Results
bacterial cytoplasm --- Certainty = 0.3031(Affirmative) <succ>
bacterial membrane --- Certainty = 0.0000(Not Clear) <succ>
bacterial outside --- Certainty = 0.0000(Not Clear) <succ>

SEQ ID 190 is predicted to be primase-related protein.

### Example 96

A DNA sequence was identified in S. agalactiae <SEQ ID 191> which encodes the amino acid sequence <SEQ ID 192>. Analysis of this protein sequence reveals the following:

Lipop: Possible site: −1 Crend: 8
McG: Discrim Score: −18.44
GvH: Signal Score (−7.5): −7.91
Possible site: 28
>>> Seems to have no N-terminal signal seq.
ALOM program count: 0 value: 13.79 threshold: 0.0
PERIPHERAL Likelihood = 13.79 46
modified ALOM score: −3.26
Final Results
bacterial cytoplasm --- Certainty = 0.4769(Affirmative) <succ>
bacterial membrane --- Certainty = 0.0000(Not Clear) <succ>
bacterial outside --- Certainty = 0.0000(Not Clear) <succ>

### Example 97

A DNA sequence was identified in S. agalactiae <SEQ ID 193> which encodes the amino acid sequence <SEQ ID 194>. Analysis of this protein sequence reveals the following:

Lipop: Possible site: −1 Crend: 10
McG: Discrim Score: −10.43
GvH: Signal Score (−7.5): −5.4
Possible site: 20
>>> Seems to have no N-terminal signal seq.
ALOM program count: 0 value: 10.98 threshold: 0.0
PERIPHERAL Likelihood = 10.98 2
modified ALOM score: −2.70
Final Results
bacterial cytoplasm --- Certainty = 0.2666(Affirmative) <succ>
bacterial membrane --- Certainty = 0.0000(Not Clear) <succ>
bacterial outside --- Certainty = 0.0000(Not Clear) <succ>

### Example 98

A DNA sequence was identified in S. agalactiae <SEQ ID 195> which encodes the amino acid sequence <SEQ ID 196>. Analysis of this protein sequence reveals the following:

Lipop: Possible site: −1 Crend: 9
McG: Discrim Score: −9.54
GvH: Signal Score (−7.5): −6.62
Possible site: 34
>>> Seems to have no N-terminal signal seq.
ALOM program count: 0 value: 11.51 threshold: 0.0
PERIPHERAL Likelihood = 11.51 22
modified ALOM score: −2.80
Final Results
bacterial cytoplasm --- Certainty = 0.2733(Affirmative) <succ>
bacterial membrane --- Certainty = 0.0000(Not Clear) <succ>
bacterial outside --- Certainty = 0.0000(Not Clear) <succ>

SEQ ID 196 is conserved.

### Example 99

A DNA sequence was identified in S. agalactiae <SEQ ID 197> which encodes the amino acid sequence <SEQ ID 198>. Analysis of this protein sequence reveals the following:

Lipop: Possible site: −1 Crend: 5
McG: Discrim Score: −9.35
GvH: Signal Score (−7.5): −8.51
Possible site: 59
>>> Seems to have no N-terminal signal seq.
ALOM program count: 0 value: 8.86 threshold: 0.0
PERIPHERAL Likelihood = 8.86 58
modified ALOM score: −2.27
Final Results
bacterial cytoplasm --- Certainty = 0.3072(Affirmative) <succ>
bacterial membrane --- Certainty = 0.0000(Not Clear) <succ>
bacterial outside --- Certainty = 0.0000(Not Clear) <succ>

SEQ ID 198 is predicted to be conserved domain protein.

### Example 100

A DNA sequence was identified in S. agalactiae <SEQ ID 199> which encodes the amino acid sequence <SEQ ID 200>. Analysis of this protein sequence reveals the following:

Lipop: Possible site: −1 Crend: 8
McG: Discrim Score: −9.31
GvH: Signal Score (−7.5): −5.47
Possible site: 36
>>> Seems to have no N-terminal signal seq.
ALOM program count: 0 value: 6.79 threshold: 0.0
PERIPHERAL Likelihood = 6.79 181
modified ALOM score: −1.86
Final Results
bacterial cytoplasm --- Certainty = 0.2456(Affirmative) <succ>
bacterial membrane --- Certainty = 0.0000(Not Clear) <succ>
bacterial outside --- Certainty = 0.0000(Not Clear) <succ>

SEQ ID 200 is conserved.

### Example 101

A DNA sequence was identified in S. agalactiae <SEQ ID 201> which encodes the amino acid sequence <SEQ ID 202>. Analysis of this protein sequence reveals the following:

Lipop: Possible site: −1 Crend: 10
McG: Discrim Score: −28.48
GvH: Signal Score (−7.5): −8.72
Possible site: 45
>>> Seems to have no N-terminal signal seq.
ALOM program count: 0 value: 12.89 threshold: 0.0
PERIPHERAL Likelihood = 12.89 30
modified ALOM score: −3.08
Final Results
bacterial cytoplasm --- Certainty = 0.6941(Affirmative) <succ>
bacterial membrane --- Certainty = 0.0000(Not Clear) <succ>
bacterial outside --- Certainty = 0.0000(Not Clear) <succ>

### Example 102

A DNA sequence was identified in S. agalactiae <SEQ ID 203> which encodes the amino acid sequence <SEQ ID 204>. Analysis of this protein sequence reveals the following:

Lipop: Possible site: −1 Crend: 7
McG: Discrim Score: −6.56
GvH: Signal Score (−7.5): −7.35
Possible site: 55
>>> Seems to have no N-terminal signal seq.
ALOM program count: 0 value: 2.97 threshold: 0.0
PERIPHERAL Likelihood = 2.97 133
modified ALOM score: −1.09
Final Results
bacterial cytoplasm --- Certainty = 0.2282(Affirmative) <succ>
bacterial membrane --- Certainty = 0.0000(Not Clear) <succ>
bacterial outside --- Certainty = 0.0000(Not Clear) <succ>

SEQ ID 204 is predicted to be ribosomal large subunit pseudouridine synthase, RluD subfamily.

### Example 103

A DNA sequence was identified in S. agalactiae <SEQ ID 205> which encodes the amino acid sequence <SEQ ID 206>. Analysis of this protein sequence reveals the following:

Lipop: Possible site: −1 Crend: 0
McG: Discrim Score: −5.41
GvH: Signal Score (−7.5): −3.42
Possible site: 45
>>> Seems to have no N-terminal signal seq.
ALOM program count: 7 value: −10.77 threshold: 0.0

| | | | |
|---|---|---|---|
| INTEGRAL | Likelihood = −10.77 | Transmembrane | 259-275 |
| | | | (255-277) |
| INTEGRAL | Likelihood = −6.90 | Transmembrane | 23-39 |
| | | | (21-41) |
| INTEGRAL | Likelihood = −6.79 | Transmembrane | 172-188 |
| | | | (166-190) |
| INTEGRAL | Likelihood = −6.37 | Transmembrane | 107-123 |
| | | | (103-125) |
| INTEGRAL | Likelihood = −5.57 | Transmembrane | 230-246 |
| | | | (222-249) |
| INTEGRAL | Likelihood = −3.40 | Transmembrane | 213-229 |
| | | | (210-229) |
| INTEGRAL | Likelihood = −0.96 | Transmembrane | 46-62 |
| | | | (45-62) |
| PERIPHERAL | Likelihood = 1.59 | 81 | |

modified ALOM score: 2.65
Final Results
bacterial membrane --- Certainty = 0.5310(Affirmative) <succ>
bacterial outside --- Certainty = 0.0000(Not Clear) <succ>
bacterial cytoplasm --- Certainty = 0.0000(Not Clear) <succ>

SEQ ID 206 is predicted to be a transporter.

### Example 104

A DNA sequence was identified in S. agalactiae <SEQ ID 207> which encodes the amino acid sequence <SEQ ID 208>. Analysis of this protein sequence reveals the following:

Lipop: Possible site: −1 Crend: 10
McG: Discrim Score: −4.99
GvH: Signal Score (−7.5): −3.84
Possible site: 52
>>> Seems to have no N-terminal signal seq.
ALOM program count: 0 value: 2.54 threshold: 0.0
PERIPHERAL Likelihood = 2.54 352
modified ALOM score: −1.01
Final Results
bacterial cytoplasm --- Certainty = 0.1265(Affirmative) <succ>
bacterial membrane --- Certainty = 0.0000(Not Clear) <succ>
bacterial outside --- Certainty = 0.0000(Not Clear) <succ>

SEQ ID 208 is predicted to be inosine-5′-monophosphate dehydrogenase (guaB).

### Example 105

A DNA sequence was identified in S. agalactiae <SEQ ID 209> which encodes the amino acid sequence <SEQ ID 210>. Analysis of this protein sequence reveals the following:

Lipop: Possible site: −1 Crend: 9
McG: Discrim Score: −8.58
GvH: Signal Score (−7.5): −5.78
Possible site: 13
>>> Seems to have no N-terminal signal seq.
ALOM program count: 0 value: 1.70 threshold: 0.0
PERIPHERAL Likelihood = 1.70 75
modified ALOM score: −0.84
Final Results
bacterial cytoplasm --- Certainty = 0.2372(Affirmative) <succ>
bacterial membrane --- Certainty = 0.0000(Not Clear) <succ>
bacterial outside --- Certainty = 0.0000(Not Clear) <succ>

SEQ ID 210 is predicted to be diacylglycerol kinase catalytic domain protein.

### Example 106

A DNA sequence was identified in S. agalactiae <SEQ ID 211> which encodes the amino acid sequence <SEQ ID 212>. Analysis of this protein sequence reveals the following:

Lipop: Possible site: −1 Crend: 9
McG: Discrim Score: −6.62
GvH: Signal Score (−7.5): −6.28
Possible site: 43
>>> Seems to have no N-terminal signal seq.
ALOM program count: 0 value: 7.48 threshold: 0.0
PERIPHERAL Likelihood = 7.48 83
modified ALOM score: −2.00
Final Results
bacterial cytoplasm --- Certainty = 0.2079(Affirmative) <succ>
bacterial membrane --- Certainty = 0.0000(Not Clear) <succ>
bacterial outside --- Certainty = 0.0000(Not Clear) <succ>

SEQ ID 212 is conserved.

### Example 107

A DNA sequence was identified in S. agalactiae <SEQ ID 213> which encodes the amino acid sequence <SEQ ID 214>. Analysis of this protein sequence reveals the following:

Lipop: Possible site: −1 Crend: 9
McG: Discrim Score: −7.71
GvH: Signal Score (−7.5): −4.11
Possible site: 46
>>> Seems to have no N-terminal signal seq.
ALOM program count: 0 value: 9.87 threshold: 0.0
PERIPHERAL Likelihood = 9.87 43
modified ALOM score: −2.47
Final Results
bacterial cytoplasm --- Certainty = 0.1865(Affirmative) <succ>
bacterial membrane --- Certainty = 0.0000(Not Clear) <succ>
bacterial outside --- Certainty = 0.0000(Not Clear) <succ>

### Example 108

A DNA sequence was identified in S. agalactiae <SEQ ID 215> which encodes the amino acid sequence <SEQ ID 216>. Analysis of this protein sequence reveals the following:

Lipop: Possible site: −1 Crend: 4
McG: Discrim Score: 4.57
GvH: Signal Score (−7.5): −5.91
Possible site: 50
>>> Seems to have an uncleavable N-term signal seq
ALOM program count: 2 value: −11.68 threshold: 0.0

| | | | |
|---|---|---|---|
| INTEGRAL | Likelihood = −11.68 | Transmembrane | 30-46 (26-53) |
| INTEGRAL | Likelihood = −0.80 | Transmembrane | 6-22 (5-23) |
| PERIPHERAL | Likelihood = 47.91 | 41 | |

modified ALOM score: 2.84
Final Results
bacterial membrane --- Certainty = 0.5670(Affirmative) <succ>
bacterial outside --- Certainty = 0.0000(Not Clear) <succ>
bacterial cytoplasm --- Certainty = 0.0000(Not Clear) <succ>

SEQ ID 216 is conserved.

### Example 109

A DNA sequence was identified in S. agalactiae <SEQ ID 217> which encodes the amino acid sequence <SEQ ID 218>. Analysis of this protein sequence reveals the following:

Lipop: Possible site: −1 Crend: 6
McG: Discrim Score: −12.87
GvH: Signal Score (−7.5): −3.7
Possible site: 40
>>> Seems to have no N-terminal signal seq.
ALOM program count: 1 value: −4.88 threshold: 0.0

| | | | |
|---|---|---|---|
| INTEGRAL | Likelihood = −4.88 | Transmembrane | 44-60 (41-61) |
| PERIPHERAL | Likelihood = 10.03 | 2 | |

modified ALOM score: 1.48
Final Results
bacterial membrane --- Certainty = 0.2954(Affirmative) <succ>
bacterial outside --- Certainty = 0.0000(Not Clear) <succ>
bacterial cytoplasm --- Certainty = 0.0000(Not Clear) <succ>

SEQ ID 218 is predicted to be Tn916.

### Example 110

A DNA sequence was identified in S. agalactiae <SEQ ID 219> which encodes the amino acid sequence <SEQ ID 220>. Analysis of this protein sequence reveals the following:

Lipop: Possible site: −1 Crend: 7
McG: Discrim Score: −3.24
GvH: Signal Score (−7.5): −12.59
Possible site: 16
>>> Seems to have no N-terminal signal seq.
ALOM program count: 0 value: 8.06 threshold: 0.0
PERIPHERAL Likelihood = 8.06 4
modified ALOM score: −2.11
Final Results
bacterial cytoplasm --- Certainty = 0.2666(Affirmative) <succ>
bacterial membrane --- Certainty = 0.0000(Not Clear) <succ>
bacterial outside --- Certainty = 0.0000(Not Clear) <succ>

SEQ ID 220 is predicted to be Tn916.

### Example 111

A DNA sequence was identified in S. agalactiae <SEQ ID 221> which encodes the amino acid sequence <SEQ ID 222>. Analysis of this protein sequence reveals the following:

Lipop: Possible site: −1 Crend: 3
McG: Discrim Score: −2.03
GvH: Signal Score (−7.5): −9.79
Possible site: 52
>>> Seems to have no N-terminal signal seq.
ALOM program count: 0 value: 11.35 threshold: 0.0
PERIPHERAL Likelihood = 11.35 40
modified ALOM score: −2.77
Final Results
bacterial cytoplasm --- Certainty = 0.1864(Affirmative) <succ>
bacterial membrane --- Certainty = 0.0000(Not Clear) <succ>
bacterial outside --- Certainty = 0.0000(Not Clear) <succ>

SEQ ID 222 is predicted to be Tn916.

### Example 112

A DNA sequence was identified in S. agalactiae <SEQ ID 223> which encodes the amino acid sequence <SEQ ID 224>. Analysis of this protein sequence reveals the following:

Lipop: Possible site: −1 Crend: 9
McG: Discrim Score: −7.39
GvH: Signal Score (−7.5): −8.64
Possible site: 18
>>> Seems to have no N-terminal signal seq.
ALOM program count: 0 value: 14.69 threshold: 0.0
PERIPHERAL Likelihood = 14.69 2
modified ALOM score: −3.44
Final Results
bacterial cytoplasm --- Certainty = 0.2706(Affirmative) <succ>
bacterial membrane --- Certainty = 0.0000(Not Clear) <succ>
bacterial outside --- Certainty = 0.0000(Not Clear) <succ>

SEQ ID 224 is predicted to be Tn916, tetM leader peptide.

### Example 113

A DNA sequence was identified in S. agalactiae <SEQ ID 225> which encodes the amino acid sequence <SEQ ID 226>. Analysis of this protein sequence reveals the following:

Lipop: Possible site: −1 Crend: 7
McG: Discrim Score: −18.13
GvH: Signal Score (−7.5): −9.17
Possible site: 20
>>> Seems to have no N-terminal signal seq.
ALOM program count: 0 value: 15.44 threshold: 0.0
PERIPHERAL Likelihood = 15.44 3
modified ALOM score: −3.59
Final Results
bacterial cytoplasm --- Certainty = 0.4960(Affirmative) <succ>
bacterial membrane --- Certainty = 0.0000(Not Clear) <succ>
bacterial outside --- Certainty = 0.0000(Not Clear) <succ>

SEQ ID 226 is predicted to be ribosomal protein L33.

### Example 114

A DNA sequence was identified in S. agalactiae <SEQ ID 227> which encodes the amino acid sequence <SEQ ID 228>. Analysis of this protein sequence reveals the following:

Lipop: Possible site: −1 Crend: 9
McG: Discrim Score: −17.04
GvH: Signal Score (−7.5): −8.31
Possible site: 22
>>> Seems to have no N-terminal signal seq.
ALOM program count: 1 value: −2.23 threshold: 0.0

| | | | |
|---|---|---|---|
| INTEGRAL | Likelihood = −2.23 | Transmembrane | 102-118 |
| | | | (101-118) |
| PERIPHERAL | Likelihood = 9.18 | 43 | |

modified ALOM score: 0.95
Final Results
bacterial membrane --- Certainty = 0.1893(Affirmative) <succ>
bacterial outside --- Certainty = 0.0000(Not Clear) <succ>
bacterial cytoplasm --- Certainty = 0.0000(Not Clear) <succ>

SEQ ID 228 is predicted to be HNH endonuclease family protein.

### Example 115

A DNA sequence was identified in S. agalactiae <SEQ ID 229> which encodes the amino acid sequence <SEQ ID 230>. Analysis of this protein sequence reveals the following:

Lipop: Possible site: −1 Crend: 4
McG: Discrim Score: −8.57
GvH: Signal Score (−7.5): −6.8
Possible site: 21
>>> Seems to have no N-terminal signal seq.
ALOM program count: 0 value: 11.94 threshold: 0.0
PERIPHERAL Likelihood = 11.94 6
modified ALOM score: −2.89
Final Results
bacterial cytoplasm --- Certainty = 0.2574(Affirmative) <succ>
bacterial membrane --- Certainty = 0.0000(Not Clear) <succ>
bacterial outside --- Certainty = 0.0000(Not Clear) <succ>

SEQ ID 230 is predicted to be ribosomal protein L33.

### Example 116

A DNA sequence was identified in S. agalactiae <SEQ ID 231> which encodes the amino acid sequence <SEQ ID 232>. Analysis of this protein sequence reveals the following:

Lipop: Possible site: −1 Crend: 0
McG: Discrim Score: −1.05
GvH: Signal Score (−7.5): 4.41
Possible site: 13
>>> Seems to have no N-terminal signal seq.
ALOM program count: 0 value: 4.51 threshold: 0.0
PERIPHERAL Likelihood = 4.51 3
modified ALOM score: −1.40
Final Results
bacterial membrane --- Certainty = 0.0000(Not Clear) <succ>
bacterial outside --- Certainty = 0.0000(Not Clear) <succ>
bacterial cytoplasm --- Certainty = 0.0000(Not Clear) <succ>

SEQ ID 232 is predicted to be conjugal transfer protein, interruption-N.

### Example 117

A DNA sequence was identified in S. agalactiae <SEQ ID 233> which encodes the amino acid sequence <SEQ ID 234>. Analysis of this protein sequence reveals the following:

GvH: Signal Score (−7.5): 4.41
Possible site: 13
>>> Seems to have no N-terminal signal seq.
ALOM program count: 0 value: 4.51 threshold: 0.0
PERIPHERAL Likelihood = 4.51 3
modified ALOM score: −1.40
Final Results
bacterial membrane --- Certainty = 0.0000(Not Clear) <succ>
bacterial outside --- Certainty = 0.0000(Not Clear) <succ>
bacterial cytoplasm --- Certainty = 0.0000(Not Clear) <succ>

SEQ ID 234 is predicted to be peptidase, M23/M37 family.

### Example 118

A DNA sequence was identified in S. pyogenes <SEQ ID 235> which encodes the amino acid sequence <SEQ ID 236>. This coding sequence was not identified by Ferretti et al.

### Example 119

A DNA sequence was identified in S. pyogenes <SEQ ID 237> which encodes the amino acid sequence <SEQ ID 238>. This coding sequence was not identified by Ferretti et al.

### Example 120

A DNA sequence was identified in S. pyogenes <SEQ ID 239> which encodes the amino acid sequence <SEQ ID 240>. This coding sequence was not identified by Ferretti et al.

### Example 121

A DNA sequence was identified in S. pyogenes <SEQ ID 241> which encodes the amino acid sequence <SEQ ID 242>. This coding sequence was not identified by Ferretti et al.

### Example 122

A DNA sequence was identified in S. pyogenes <SEQ ID 243> which encodes the amino acid sequence <SEQ ID 244>. This coding sequence was not identified by Ferretti et al.

### Example 123

A DNA sequence was identified in S. pyogenes <SEQ ID 245> which encodes the amino acid sequence <SEQ ID 246>. This coding sequence was not identified by Ferretti et al.

### Example 124

A DNA sequence was identified in S. pyogenes <SEQ ID 247> which encodes the amino acid sequence <SEQ ID 248>. This coding sequence was not identified by Ferretti et al. It may be a transposase.

### Example 125

A DNA sequence was identified in S. pyogenes <SEQ ID 249> which encodes the amino acid sequence <SEQ ID 250>. This coding sequence was not identified by Ferretti et al. It may be a transposase.

### Example 126

A DNA sequence was identified in S. pyogenes <SEQ ID 251> which encodes the amino acid sequence <SEQ ID 252>. This coding sequence was not identified by Ferretti et al.

### Example 127

A DNA sequence was identified in S. pyogenes <SEQ ID 253> which encodes the amino acid sequence <SEQ ID 254>. This coding sequence was not identified by Ferretti et al.

### Example 128

A DNA sequence was identified in S. pyogenes <SEQ ID 255> which encodes the amino acid sequence <SEQ ID 256>. This coding sequence was not identified by Ferretti et al.

### Example 129

A DNA sequence was identified in S. pyogenes <SEQ ID 257> which encodes the amino acid sequence <SEQ ID 258>. This coding sequence was not identified by Ferretti et al.

### Example 130

A DNA sequence was identified in S. pyogenes <SEQ ID 259> which encodes the amino acid sequence <SEQ ID 260>. This coding sequence was not identified by Ferretti et al.

### Example 131

A DNA sequence was identified in S. pyogenes <SEQ ID 261> which encodes the amino acid sequence <SEQ ID 262>. This coding sequence was not identified by Ferretti et al.

### Example 132

A DNA sequence was identified in S. pyogenes <SEQ ID 263> which encodes the amino acid sequence <SEQ ID 264>. This coding sequence was not identified by Ferretti et al.

### Example 133

A DNA sequence was identified in S. pyogenes <SEQ ID 265> which encodes the amino acid sequence <SEQ ID 266>. This coding sequence was not identified by Ferretti et al.

### Example 134

A DNA sequence was identified in S. pyogenes <SEQ ID 267> which encodes the amino acid sequence <SEQ ID 268>. This coding sequence was not identified by Ferretti et al. It may be an ABC transporter.

### Example 135

A DNA sequence was identified in S. pyogenes <SEQ ID 269> which encodes the amino acid sequence <SEQ ID 270>. This coding sequence was not identified by Ferretti et al. It may be an integrase.

### Example 136

A DNA sequence was identified in S. pyogenes <SEQ ID 271> which encodes the amino acid sequence <SEQ ID 272>. This coding sequence was not identified by Ferretti et al.

### Example 137

A DNA sequence was identified in S. pyogenes <SEQ ID 273> which encodes the amino acid sequence <SEQ ID 274>. This coding sequence was not identified by Ferretti et al.

### Example 138

A DNA sequence was identified in S. pyogenes <SEQ ID 275> which encodes the amino acid sequence <SEQ ID 276>. This coding sequence was not identified by Ferretti et al.

### Example 139

A DNA sequence was identified in S. pyogenes <SEQ ID 277> which encodes the amino acid sequence <SEQ ID 278>. This coding sequence was not identified by Ferretti et al.

### Example 140

A DNA sequence was identified in S. pyogenes <SEQ ID 279> which encodes the amino acid sequence <SEQ ID 280>. This coding sequence was not identified by Ferretti et al.

### Example 141

A DNA sequence was identified in S. pyogenes <SEQ ID 281> which encodes the amino acid sequence <SEQ ID 282>. This coding sequence was not identified by Ferretti et al.

### Example 142

A DNA sequence was identified in S. pyogenes <SEQ ID 283> which encodes the amino acid sequence <SEQ ID 284>. This coding sequence was not identified by Ferretti et al.

### Example 143

A DNA sequence was identified in S. pyogenes <SEQ ID 285> which encodes the amino acid sequence <SEQ ID 286>. This coding sequence was not identified by Ferretti et al.

### Example 144

A DNA sequence was identified in S. pyogenes <SEQ ID 287> which encodes the amino acid sequence <SEQ ID 288>. This coding sequence was not identified by Ferretti et al.

### Example 145

A DNA sequence was identified in S. pyogenes <SEQ ID 289> which encodes the amino acid sequence <SEQ ID 290>. This coding sequence was not identified by Ferretti et al.

### Example 146

A DNA sequence was identified in S. pyogenes <SEQ ID 291> which encodes the amino acid sequence <SEQ ID 292>. This coding sequence was not identified by Ferretti et al.

### Example 147

A DNA sequence was identified in S. pyogenes <SEQ ID 293> which encodes the amino acid sequence <SEQ ID 294>. This coding sequence was not identified by Ferretti et al.

### Example 148

A DNA sequence was identified in S. pyogenes <SEQ ID 295> which encodes the amino acid sequence <SEQ ID 296>. This coding sequence was not identified by Ferretti et al.

### Example 149

A DNA sequence was identified in S. pyogenes <SEQ ID 297> which encodes the amino acid sequence <SEQ ID 298>. This coding sequence was not identified by Ferretti et al.

### Example 150

A DNA sequence was identified in S. pyogenes <SEQ ID 299> which encodes the amino acid sequence <SEQ ID 300>. This coding sequence was not identified by Ferretti et al. It may be a regulatory protein.

### Example 151

A DNA sequence was identified in S. pyogenes <SEQ ID 301> which encodes the amino acid sequence <SEQ ID 302>. This coding sequence is frameshifted relative to G1:13621570.

### Example 152

A DNA sequence was identified in S. pyogenes <SEQ ID 303> which encodes the amino acid sequence <SEQ ID 304>. This coding sequence is frameshifted relative to G1:13621491.

### Example 153

A DNA sequence was identified in S. pyogenes <SEQ ID 305> which encodes the amino acid sequence <SEQ ID 306>. This coding sequence was not identified by Ferretti et al. It may be a metal-dependent transcription regulator.

### Example 154

A DNA sequence was identified in S. pyogenes <SEQ ID 307> which encodes the amino acid sequence <SEQ ID 308>. This coding sequence was not identified by Ferretti et al.

### Example 155

A DNA sequence was identified in S. pyogenes <SEQ ID 309> which encodes the amino acid sequence <SEQ ID 310>. This coding sequence was not identified by Ferretti et al.

### Example 156

A DNA sequence was identified in S. pyogenes <SEQ ID 311> which encodes the amino acid sequence <SEQ ID 312>. This coding sequence was not identified by Ferretti et al. It may be a maltose/maltodextrin binding protein.

### Example 157

A DNA sequence was identified in S. pyogenes <SEQ ID 313> which encodes the amino acid sequence <SEQ ID 314>. This coding sequence was not identified by Ferretti et al. It may be a beta-glucosidaase.

### Example 158

A DNA sequence was identified in S. pyogenes <SEQ ID 315> which encodes the amino acid sequence <SEQ ID 316>. This coding sequence was not identified by Ferretti et al.

### Example 159

A DNA sequence was identified in S. pyogenes <SEQ ID 317> which encodes the amino acid sequence <SEQ ID 318>. This coding sequence was not identified by Ferretti et al. It may be a sugar-binding transport protein

### Example 160

A DNA sequence was identified in S. pyogenes <SEQ ID 319> which encodes the amino acid sequence <SEQ ID 320>. This coding sequence was not identified by Ferretti et al. It may be a transcription regulator.

### Example 161

A DNA sequence was identified in S. pyogenes <SEQ ID 321> which encodes the amino acid sequence <SEQ ID 322>. This coding sequence was not identified by Ferretti et al. It may be an ABC transporter.

### Example 162

A DNA sequence was identified in S. pyogenes <SEQ ID 323> which encodes the amino acid sequence <SEQ ID 324>. This coding sequence was not identified by Ferretti et al. It may be a phospho-beta-D-galactosidase.

### Example 163

A DNA sequence was identified in S. pyogenes <SEQ ID 325> which encodes the amino acid sequence <SEQ ID 326>. This coding sequence was not identified by Ferretti et al. It may be a mitogenic factor.

### Example 164

A DNA sequence was identified in S. pyogenes <SEQ ID 327> which encodes the amino acid sequence <SEQ ID 328>. This coding sequence was not identified by Ferretti et al.

### Example 165

A DNA sequence was identified in S. pyogenes <SEQ ID 329> which encodes the amino acid sequence <SEQ ID 330>. This coding sequence was not identified by Ferretti et al. It may be a transposase.

### Example 166

A DNA sequence was identified in S. pyogenes <SEQ ID 331> which encodes the amino acid sequence <SEQ ID 332>. This coding sequence was not identified by Ferretti et al.

### Example 167

A DNA sequence was identified in S. pyogenes <SEQ ID 333> which encodes the amino acid sequence <SEQ ID 334>. This coding sequence was not identified by Ferretti et al. It may be a positive regulator.

### Example 168

A DNA sequence was identified in S. pyogenes <SEQ ID 335> which encodes the amino acid sequence <SEQ ID 336>. This coding sequence was not identified by Ferretti et al. It may be a member of the DEAD box helicase family and may be phage-related.

### Example 169

A DNA sequence was identified in S. pyogenes <SEQ ID 337> which encodes the amino acid sequence <SEQ ID 338>. This coding sequence was not identified by Ferretti et al. and may be phage-related.

### Example 170

A DNA sequence was identified in S. pyogenes <SEQ ID 339> which encodes the amino acid sequence <SEQ ID 340>. This coding sequence was not identified by Ferretti et al. It may be a transcriptional regulator in the GntR family.

### Example 171

A DNA sequence was identified in S. pyogenes <SEQ ID 341> which encodes the amino acid sequence <SEQ ID 342>. This coding sequence was not identified by Ferretti et al. It may be a folyl-polyglutamate synthetase.

### Example 172

A DNA sequence was identified in S. pyogenes <SEQ ID 343> which encodes the amino acid sequence <SEQ ID 344>. This coding sequence was not identified by Ferretti et al.

### Example 173

A DNA sequence was identified in S. pyogenes <SEQ ID 345> which encodes the amino acid sequence <SEQ ID 346>. This coding sequence was not identified by Ferretti et al.

### Example 174

A DNA sequence was identified in S. pyogenes <SEQ ID 347> which encodes the amino acid sequence <SEQ ID 348>. This coding sequence was identified by Ferretti et al. as a putative transcription factor in the LacI family, but its antigenicity was not.

### Example 175

A DNA sequence was identified in S. pyogenes <SEQ ID 349> which encodes the amino acid sequence <SEQ ID 350>. This coding sequence was identified by Ferretti et al. as a phage-associated protein, but its antigenicity was not.

### Example 176

A DNA sequence was identified in S. pyogenes <SEQ ID 351> which encodes the amino acid sequence <SEQ ID 352>. This coding sequence was identified by Ferretti et al. as a phage-associated protein, but its antigenicity was not.

### Example 177

A DNA sequence was identified in S. pyogenes <SEQ ID 353>. The sequence encodes the amino acid sequence <SEQ ID 354>. The encoded polypeptide may be phage-associated.

### Example 178

A DNA sequence was identified in S. pyogenes <SEQ ID 355>. The sequence encodes the amino acid sequence <SEQ ID 356>. The encoded polypeptide is a transcriptional regulator (GntR family).

### Example 179

A DNA sequence was identified in S. pyogenes <SEQ ID 357>. The sequence encodes the amino acid sequence <SEQ ID 358>.

### Example 180

A DNA sequence was identified in S. pyogenes <SEQ ID 359>. The sequence encodes the amino acid sequence <SEQ ID 360>. The encoded polypeptide is a histidine triad (HIT) protein.

### Example 181

A DNA sequence was identified in S. pyogenes <SEQ ID 361>. The sequence encodes the amino acid sequence <SEQ ID 362>. The encoded polypeptide is a transcription regulator.

### Example 182

A DNA sequence was identified in S. pyogenes <SEQ ID 363>. The sequence encodes the amino acid sequence <SEQ ID 364>. The encoded polypeptide is a response regulator of salavaricin regulon.

### Example 183

A DNA sequence was identified in S. pyogenes <SEQ ID 365>. The sequence encodes the amino acid sequence <SEQ ID 366>.

### Example 184

A DNA sequence was identified in S. pyogenes <SEQ ID 367>. The sequence encodes the amino acid sequence <SEQ ID 368>. The encoded polypeptide is a shikimate 5-dehydrogenase.

### Example 185

A DNA sequence was identified in S. pyogenes <SEQ ID 369>. The sequence encodes the amino acid sequence <SEQ ID 370>.

### Example 186

A DNA sequence was identified in S. pyogenes <SEQ ID 371>. The sequence encodes the amino acid sequence <SEQ ID 372>. The encoded polypeptide is a cI-like repressor, phage associated.

### Example 187

A DNA sequence was identified in S. pyogenes <SEQ ID 373>. The sequence encodes the amino acid sequence <SEQ ID 374>. The encoded polypeptide is an acetyl-CoA:acetyltransferase.

### Example 188

A DNA sequence was identified in S. pyogenes <SEQ ID 375>. The sequence encodes the amino acid sequence <SEQ ID 376>. The encoded polypeptide is a methylmalonyl-CoA decarboxylase, gamma-subunit.

### Example 189

A DNA sequence was identified in S. pyogenes <SEQ ID 377>. The sequence encodes the amino acid sequence <SEQ ID 378>. The encoded polypeptide is an acetyl-CoA acetyltransferase.

### Example 190

A DNA sequence was identified in S. pyogenes <SEQ ID 379>. The sequence encodes the amino acid sequence <SEQ ID 380>. The encoded polypeptide is a decarboxylase, gamma chain.

### Example 191

A DNA sequence was identified in S. pyogenes <SEQ ID 381>. The sequence encodes the amino acid sequence <SEQ ID 382>. The encoded polypeptide is an ABC transporter (ATP binding)—lantibiotic associated.

### Example 192

A DNA sequence was identified in S. pyogenes <SEQ ID 383>. The sequence encodes the amino acid sequence <SEQ ID 384>. The encoded polypeptide is a transaldolase.

### Example 193

A DNA sequence was identified in S. pyogenes <SEQ ID 385>. The sequence encodes the amino acid sequence <SEQ ID 386>.

### Example 194

A DNA sequence was identified in S. pyogenes <SEQ ID 387>. The sequence encodes the amino acid sequence <SEQ ID 388>. The encoded polypeptide may be phage-associated.

### Example 195

A DNA sequence was identified in S. pyogenes <SEQ ID 389>. The sequence encodes the amino acid sequence <SEQ ID 390>. The encoded polypeptide is an esterase.

### Example 196

A DNA sequence was identified in S. pyogenes <SEQ ID 391>. The sequence encodes the amino acid sequence <SEQ ID 392>. The encoded polypeptide is a DNA binding regulatory protein—lantibiotic associated.

### Example 197

A DNA sequence was identified in S. pyogenes <SEQ ID 393>. The sequence encodes the amino acid sequence <SEQ ID 394>. The encoded polypeptide is a Xaa-His dipeptidase.

### Example 198

A DNA sequence was identified in S. pyogenes <SEQ ID 395>. The sequence encodes the amino acid sequence <SEQ ID 396>.

### Example 199

A DNA sequence was identified in S. pyogenes <SEQ ID 397>. The sequence encodes the amino acid sequence <SEQ ID 398>. The encoded polypeptide is a PTS enzyme III.

### Example 200

A DNA sequence was identified in S. pyogenes <SEQ ID 399>. The sequence encodes the amino acid sequence <SEQ ID 400>. The encoded polypeptide is a salivaricin an ABC transporter (ATP-binding protein).

### Example 201

A DNA sequence was identified in S. pyogenes <SEQ ID 401>. The sequence encodes the amino acid sequence <SEQ ID 402>. The encoded polypeptide is a PTS system, lactose-specific component IIA.

### Example 202

A DNA sequence was identified in S. pyogenes <SEQ ID 403>. The sequence encodes the amino acid sequence <SEQ ID 404>. The encoded polypeptide is a Cro-like repressor protein—phage associated.

### Example 203

A DNA sequence was identified in S. pyogenes <SEQ ID 405>. The sequence encodes the amino acid sequence <SEQ ID 406>. The encoded polypeptide is a beta-glucosidase.

### Example 204

A DNA sequence was identified in S. pyogenes <SEQ ID 407>. The sequence encodes the amino acid sequence <SEQ ID 408>. The encoded polypeptide is a M protein trans-acting positive regulator.

### Example 205

A DNA sequence was identified in S. pyogenes <SEQ ID 409>. The sequence encodes the amino acid sequence <SEQ ID 410>. The encoded polypeptide is a streptolysin S associated protein.

### Example 206

A DNA sequence was identified in S. pyogenes <SEQ ID 411>. The sequence encodes the amino acid sequence <SEQ ID 412>. The encoded polypeptide is an integrase—phage associated.

### Example 207

A DNA sequence was identified in S. pyogenes <SEQ ID 413>. The sequence encodes the amino acid sequence <SEQ ID 414>.

### Example 208

A DNA sequence was identified in S. pyogenes <SEQ ID 415>. The sequence encodes the amino acid sequence <SEQ ID 416>.

### Example 209

A DNA sequence was identified in S. pyogenes <SEQ ID 417>. The sequence encodes the amino acid sequence <SEQ ID 418>. The encoded polypeptide is a metal-dependent transcriptional regulator.

### Example 210

A DNA sequence was identified in S. pyogenes <SEQ ID 419>. The sequence encodes the amino acid sequence <SEQ ID 420>.

### Example 211

A DNA sequence was identified in S. pyogenes <SEQ ID 421>. The sequence encodes the amino acid sequence <SEQ ID 422>. The encoded polypeptide is a galactosidase acetyltransferase.

### Example 212

A DNA sequence was identified in S. pyogenes <SEQ ID 423>. The sequence encodes the amino acid sequence <SEQ ID 424>. The encoded polypeptide is a repressor protein—phage associated.

### Example 213

A DNA sequence was identified in S. pyogenes <SEQ ID 425>. The sequence encodes the amino acid sequence <SEQ ID 426>. The encoded polypeptide is an arginine repressor.

### Example 214

A DNA sequence was identified in S. pyogenes <SEQ ID 427>. The sequence encodes the amino acid sequence <SEQ ID 428>.

### Example 215

A DNA sequence was identified in S. pyogenes <SEQ ID 429>. The sequence encodes the amino acid sequence <SEQ ID 430>.

### Example 216

A DNA sequence was identified in S. pyogenes <SEQ ID 431>. The sequence encodes the amino acid sequence <SEQ ID 432>.

### Example 217

A DNA sequence was identified in S. pyogenes <SEQ ID 433>. The sequence encodes the amino acid sequence <SEQ ID 434>. The encoded polypeptide may be phage-associated.

### Example 218

A DNA sequence was identified in S. pyogenes <SEQ ID 435>. The sequence encodes the amino acid sequence <SEQ ID 436>, which is related to SEQ ID 9430 of WO02/34771. The encoded polypeptide is a 30S ribosomal protein S12.

### Example 219

A DNA sequence was identified in S. pyogenes <SEQ ID 437>. The sequence encodes the amino acid sequence <SEQ ID 438>, which is related to SEQ ID 32 of WO02/34771. The encoded polypeptide is a transposase—IS861 associated.

### Example 220

A DNA sequence was identified in S. pyogenes <SEQ ID 439>. The sequence encodes the amino acid sequence <SEQ ID 440>, which is related to SEQ ID 5756 of WO02/34771. The encoded polypeptide is a 50S ribosomal protein L34.

### Example 221

A DNA sequence was identified in S. pyogenes <SEQ ID 441>. The sequence encodes the amino acid sequence <SEQ ID 442>, which is related to SEQ ID 7334 of WO02/34771.

### Example 222

A DNA sequence was identified in S. pyogenes <SEQ ID 443>. The sequence encodes the amino acid sequence <SEQ ID 444>, which is related to SEQ ID 732 of WO02/34771.

### Example 223

A DNA sequence was identified in S. pyogenes <SEQ ID 445>. The sequence encodes the amino acid sequence <SEQ ID 446>, which is related to SEQ ID 6354 of WO02/34771. The encoded polypeptide is a 30S ribosomal protein S14.

### Example 224

A DNA sequence was identified in S. pyogenes <SEQ ID 447>. The sequence encodes the amino acid sequence <SEQ ID 448>, which is related to SEQ ID 7252 of WO02/34771.

### Example 225

A DNA sequence was identified in S. pyogenes <SEQ ID 449>. The sequence encodes the amino acid sequence <SEQ ID 450>, which is related to SEQ ID 7776 of WO02/34771.

### Example 226

A DNA sequence was identified in S. pyogenes <SEQ ID 451>. The sequence encodes the amino acid sequence <SEQ ID 452>, which is related to SEQ ID 8214 of WO02/34771.

### Example 227

A DNA sequence was identified in S. pyogenes <SEQ ID 453>. The sequence encodes the amino acid sequence <SEQ ID 454>, which is related to SEQ ID 4696 of WO02/34771. The encoded polypeptide is a 30S ribosomal protein S20.

### Example 228

A DNA sequence was identified in S. pyogenes <SEQ ID 455>. The sequence encodes the amino acid sequence <SEQ ID 456>, which is related to SEQ ID 7594 of WO02/34771.

### Example 229

A DNA sequence was identified in S. pyogenes <SEQ ID 457>. The sequence encodes the amino acid sequence <SEQ ID 458>, which is related to SEQ ID 8022 of WO02/34771.

### Example 230

A DNA sequence was identified in S. pyogenes <SEQ ID 459>. The sequence encodes the amino acid sequence <SEQ ID 460>, which is related to SEQ ID 7700 of WO02/34771.

### Example 231

A DNA sequence was identified in S. pyogenes <SEQ ID 461>. The sequence encodes the amino acid sequence <SEQ ID 462>, which is related to SEQ ID 8056 of WO02/34771. The encoded polypeptide may be phage-associated.

### Example 232

A DNA sequence was identified in S. pyogenes <SEQ ID 463>. The sequence encodes the amino acid sequence <SEQ ID 464>, which is related to SEQ ID 4438 of WO02/34771.

### Example 233

A DNA sequence was identified in S. pyogenes <SEQ ID 465>. The sequence encodes the amino acid sequence <SEQ ID 466>, which is related to SEQ ID 1124 of WO02/34771.

### Example 234

A DNA sequence was identified in S. pyogenes <SEQ ID 467>. The sequence encodes the amino acid sequence <SEQ ID 468>, which is related to SEQ ID 7840 of WO02/34771.

### Example 235

A DNA sequence was identified in S. pyogenes <SEQ ID 469>. The sequence encodes the amino acid sequence <SEQ ID 470>, which is related to SEQ ID 8164 of WO02/34771.

### Example 236

A DNA sequence was identified in S. pyogenes <SEQ ID 471>. The sequence encodes the amino acid sequence <SEQ ID 472>, which is related to SEQ ID 2374 of WO02/34771.

### Example 237

A DNA sequence was identified in S. pyogenes <SEQ ID 473>. The sequence encodes the amino acid sequence <SEQ ID 474>, which is related to SEQ ID 7266 of WO02/34771.

### Example 238

A DNA sequence was identified in S. pyogenes <SEQ ID 475>. The sequence encodes the amino acid sequence <SEQ ID 476>, which is related to SEQ ID 7338 of WO02/34771. The encoded polypeptide is a regulatory protein.

### Example 239

A DNA sequence was identified in S. pyogenes <SEQ ID 477>. The sequence encodes the amino acid sequence <SEQ ID 478>, which is related to SEQ ID 3486 of WO02/34771. The encoded polypeptide is a ribosomal protein.

### Example 240

A DNA sequence was identified in S. pyogenes <SEQ ID 479>. The sequence encodes the amino acid sequence <SEQ ID 480>, which is related to SEQ ID 2692 of WO02/34771.

### Example 241

A DNA sequence was identified in S. pyogenes <SEQ ID 481>. The sequence encodes the amino acid sequence <SEQ ID 482>, which is related to SEQ ID 2180 of WO02/34771.

### Example 242

A DNA sequence was identified in S. pyogenes <SEQ ID 483>. The sequence encodes the amino acid sequence <SEQ ID 484>, which is related to SEQ ID 7560 of WO02/34771.

### Example 243

A DNA sequence was identified in S. pyogenes <SEQ ID 485>. The sequence encodes the amino acid sequence <SEQ ID 486>, which is related to SEQ ID 1428 of WO02/34771. The encoded polypeptide may be phage associated.

### Example 244

A DNA sequence was identified in S. pyogenes <SEQ ID 487>. The sequence encodes the amino acid sequence <SEQ ID 488>, which is related to SEQ ID 604 of WO02/34771.

### Example 245

A DNA sequence was identified in S. pyogenes <SEQ ID 489>. The sequence encodes the amino acid sequence <SEQ ID 490>, which is related to SEQ ID 6342 of WO02/34771. The encoded polypeptide is a 505 ribosomal protein L18.

### Example 246

A DNA sequence was identified in S. pyogenes <SEQ ID 491>. The sequence encodes the amino acid sequence <SEQ ID 492>, which is related to SEQ ID 6992 of WO02/34771. The encoded polypeptide is a 30S ribosomal protein S11.

### Example 247

A DNA sequence was identified in S. pyogenes <SEQ ID 493>. The sequence encodes the amino acid sequence <SEQ ID 494>, which is related to SEQ ID 850 of WO02/34771. The encoded polypeptide is a ribonuclease P protein component.

### Example 248

A DNA sequence was identified in S. pyogenes <SEQ ID 495>. The sequence encodes the amino acid sequence <SEQ ID 496>, which is related to SEQ ID 1048 of WO02/34771.

### Example 249

A DNA sequence was identified in S. pyogenes <SEQ ID 497>. The sequence encodes the amino acid sequence <SEQ ID 498>, which is related to SEQ ID 5904 of WO02/34771.

### Example 250

A DNA sequence was identified in S. pyogenes <SEQ ID 499>. The sequence encodes the amino acid sequence <SEQ ID 500>, which is related to SEQ ID 4164 of WO02/34771.

### Example 251

A DNA sequence was identified in S. pyogenes <SEQ ID 501>. The sequence encodes the amino acid sequence <SEQ ID 502>, which is related to SEQ ID 1458 of WO02/34771. The encoded polypeptide may be phage-associated.

### Example 252

A DNA sequence was identified in S. pyogenes <SEQ ID 503>. The sequence encodes the amino acid sequence <SEQ ID 504>, which is related to SEQ ID 740 of WO02/34771.

### Example 253

A DNA sequence was identified in S. pyogenes <SEQ ID 505>. The sequence encodes the amino acid sequence <SEQ ID 506>, which is related to SEQ ID 7490 of WO02/34771.

### Example 254

A DNA sequence was identified in S. pyogenes <SEQ ID 507>. The sequence encodes the amino acid sequence <SEQ ID 508>, which is related to SEQ ID 5180 of WO02/34771. The encoded polypeptide is a transcriptional regulator (MarR family).

### Example 255

A DNA sequence was identified in S. pyogenes <SEQ ID 509>. The sequence encodes the amino acid sequence <SEQ ID 510>, which is related to SEQ ID 400 of WO02/34771. The encoded polypeptide is a competence protein.

### Example 256

A DNA sequence was identified in S. pyogenes <SEQ ID 511>. The sequence encodes the amino acid sequence <SEQ ID 512>, which is related to SEQ ID 3526 of WO02/34771.

### Example 257

A DNA sequence was identified in S. pyogenes <SEQ ID 513>. The sequence encodes the amino acid sequence <SEQ ID 514>, which is related to SEQ ID 7576 of WO02/34771.

### Example 258

A DNA sequence was identified in S. pyogenes <SEQ ID 515>. The sequence encodes the amino acid sequence <SEQ ID 516>, which is related to SEQ ID 7778 of WO02/34771.

### Example 259

A DNA sequence was identified in S. pyogenes <SEQ ID 517>. The sequence encodes the amino acid sequence <SEQ ID 518>, which is related to SEQ ID 1432 of WO02/34771. The encoded polypeptide may be phage-associated.

### Example 260

A DNA sequence was identified in S. pyogenes <SEQ ID 519>. The sequence encodes the amino acid sequence <SEQ ID 520>, which is related to SEQ ID 8062 of WO02/34771. The encoded polypeptide may be phage-associated.

### Example 261

A DNA sequence was identified in S. pyogenes <SEQ ID 521>. The sequence encodes the amino acid sequence <SEQ ID 522>, which is related to SEQ ID 8068 of WO02/34771. The encoded polypeptide may be phage-associated.

### Example 262

A DNA sequence was identified in S. pyogenes <SEQ ID 523>. The sequence encodes the amino acid sequence <SEQ ID 524>, which is related to SEQ ID 552 of WO02/34771.

### Example 263

A DNA sequence was identified in S. pyogenes <SEQ ID 525>. The sequence encodes the amino acid sequence <SEQ ID 526>, which is related to SEQ ID 7342 of WO02/34771.

### Example 264

A DNA sequence was identified in S. pyogenes <SEQ ID 527>. The sequence encodes the amino acid sequence <SEQ ID 528>, which is related to SEQ ID 4842 of WO02/34771.

### Example 265

A DNA sequence was identified in S. pyogenes <SEQ ID 529>. The sequence encodes the amino acid sequence <SEQ ID 530>, which is related to SEQ ID 7076 of WO02/34771. The encoded polypeptide is a translation initiation factor 3 (IF3).

### Example 266

A DNA sequence was identified in S. pyogenes <SEQ ID 531>. The sequence encodes the amino acid sequence <SEQ ID 532>, which is related to SEQ ID 2806 of WO02/34771.

### Example 267

A DNA sequence was identified in S. pyogenes <SEQ ID 533>. The sequence encodes the amino acid sequence <SEQ ID 534>, which is related to SEQ ID 1582 of WO02/34771. The encoded polypeptide is a PTS system, enzyme IIA component.

### Example 268

A DNA sequence was identified in S. pyogenes <SEQ ID 535>. The sequence encodes the amino acid sequence <SEQ ID 536>, which is related to SEQ ID 5204 of WO02/34771. The encoded polypeptide is a biotoin carboxyl carrier protein.

### Example 269

A DNA sequence was identified in S. pyogenes <SEQ ID 537>. The sequence encodes the amino acid sequence <SEQ ID 538>, which is related to SEQ ID 6314 of WO02/34771. The encoded polypeptide is a topology modulator.

### Example 270

A DNA sequence was identified in S. pyogenes <SEQ ID 539>. The sequence encodes the amino acid sequence <SEQ ID 540>, which is related to SEQ ID 5720 of WO02/34771.

### Example 271

A DNA sequence was identified in S. pyogenes <SEQ ID 541>. The sequence encodes the amino acid sequence <SEQ ID 542>, which is related to SEQ ID 6118 of WO02/34771. The encoded polypeptide is a protein involved in lantibiotic (srt) production.

### Example 272

A DNA sequence was identified in S. pyogenes <SEQ ID 543>. The sequence encodes the amino acid sequence <SEQ ID 544>, which is related to SEQ ID 7042 of WO02/34771. The encoded polypeptide is a DNA-directed RNA polymerase (delta subunit).

### Example 273

A DNA sequence was identified in S. pyogenes <SEQ ID 545>. The sequence encodes the amino acid sequence <SEQ ID 546>, which is related to SEQ ID 6900 of WO02/34771.

### Example 274

A DNA sequence was identified in S. pyogenes <SEQ ID 547>. The sequence encodes the amino acid sequence <SEQ ID 548>, which is related to SEQ ID 7308 of WO02/34771. The encoded polypeptide is a V-type Na⁺-ATPase subunit E.

### Example 275

A DNA sequence was identified in S. pyogenes <SEQ ID 549>. The sequence encodes the amino acid sequence <SEQ ID 550>, which is related to SEQ ID 932 of WO02/34771.

### Example 276

A DNA sequence was identified in S. pyogenes <SEQ ID 551>. The sequence encodes the amino acid sequence <SEQ ID 552>, which is related to SEQ ID 7698 of WO02/34771.

### Example 277

A DNA sequence was identified in S. pyogenes <SEQ ID 553>. The sequence encodes the amino acid sequence <SEQ ID 554>, which is related to SEQ ID 9074 of WO02/34771. The encoded polypeptide is a peptidoglycan hydrolase.

### Example 278

A DNA sequence was identified in S. pyogenes <SEQ ID 555>. The sequence encodes the amino acid sequence <SEQ ID 556>, which is related to SEQ ID 5276 of WO02/34771.

### Example 279

A DNA sequence was identified in S. pyogenes <SEQ ID 557>. The sequence encodes the amino acid sequence <SEQ ID 558>, which is related to SEQ ID 6578 of WO02/34771. The encoded polypeptide is a thymidine kinase.

### Example 280

A DNA sequence was identified in S. pyogenes <SEQ ID 559>. The sequence encodes the amino acid sequence <SEQ ID 560>, which is related to SEQ ID 5392 of WO02/34771. The encoded polypeptide is a transcriptional regulator.

### Example 281

A DNA sequence was identified in S. pyogenes <SEQ ID 561>. The sequence encodes the amino acid sequence <SEQ ID 562>, which is related to SEQ ID 6826 of WO02/34771. The encoded polypeptide is a recombination protein.

### Example 282

A DNA sequence was identified in S. pyogenes <SEQ ID 563>. The sequence encodes the amino acid sequence <SEQ ID 564>, which is related to SEQ ID 8426 of WO02/34771.

### Example 283

A DNA sequence was identified in S. pyogenes <SEQ ID 565>. The sequence encodes the amino acid sequence <SEQ ID 566>, which is related to SEQ ID 5824 of WO02/34771. The encoded polypeptide is a myo-inositol-1(or 4)-monophosphatase.

### Example 284

A DNA sequence was identified in S. pyogenes <SEQ ID 567>. The sequence encodes the amino acid sequence <SEQ ID 568>, which is related to SEQ ID 6260 of WO02/34771.

### Example 285

A DNA sequence was identified in S. pyogenes <SEQ ID 569>. The sequence encodes the amino acid sequence <SEQ ID 570>, which is related to SEQ ID 6392 of WO02/34771. The encoded polypeptide is a 50S ribosomal protein L2.

### Example 286

A DNA sequence was identified in S. pyogenes <SEQ ID 571>. The sequence encodes the amino acid sequence <SEQ ID 572>, which is related to SEQ ID 3740 of WO02/34771. The encoded polypeptide is a fimbria-associated protein.

### Example 287

A DNA sequence was identified in S. pyogenes <SEQ ID 573>. The sequence encodes the amino acid sequence <SEQ ID 574>, which is related to SEQ ID 846 of WO02/34771.

### Example 288

A DNA sequence was identified in S. pyogenes <SEQ ID 575>. The sequence encodes the amino acid sequence <SEQ ID 576>, which is related to SEQ ID 80. of WO02/34771. The encoded polypeptide is a uridylate kinase (UMP-kinase).

### Example 289

A DNA sequence was identified in S. pyogenes <SEQ ID 577>. The sequence encodes the amino acid sequence <SEQ ID 578>, which is related to SEQ ID 7542 of WO02/34771.

### Example 290

A DNA sequence was identified in S. pyogenes <SEQ ID 579>. The sequence encodes the amino acid sequence <SEQ ID 580>, which is related to SEQ ID 5508 of WO02/34771.

### Example 291

A DNA sequence was identified in S. pyogenes <SEQ ID 581>. The sequence encodes the amino acid sequence <SEQ ID 582>, which is related to SEQ ID 3780 of WO02/34771. The encoded polypeptide is a glycosyl transferase.

### Example 292

A DNA sequence was identified in S. pyogenes <SEQ ID 583>. The sequence encodes the amino acid sequence <SEQ ID 584>, which is related to SEQ ID 4220 of WO02/34771.

### Example 293

A DNA sequence was identified in S. pyogenes <SEQ ID 585>. The sequence encodes the amino acid sequence <SEQ ID 586>, which is related to SEQ ID 1922 of WO02/34771. The encoded polypeptide is a DNA processing protein (Smf family).

### Example 294

A DNA sequence was identified in S. pyogenes <SEQ ID 587>. The sequence encodes the amino acid sequence <SEQ ID 588>, which is related to SEQ ID 6172 of WO02/34771.

### Example 295

A DNA sequence was identified in S. pyogenes <SEQ ID 589>. The sequence encodes the amino acid sequence <SEQ ID 590>, which is related to SEQ ID 8010 of WO02/34771.

### Example 296

A DNA sequence was identified in S. pyogenes <SEQ ID 591>. The sequence encodes the amino acid sequence <SEQ ID 592>, which is related to SEQ ID 6722 of WO02/34771. The encoded polypeptide is a 1-acylglycerol-3-phosphate O-acyltransferase.

### Example 297

A DNA sequence was identified in S. pyogenes <SEQ ID 593>. The sequence encodes the amino acid sequence <SEQ ID 594>, which is related to SEQ ID 3136 of WO02/34771.

### Example 298

A DNA sequence was identified in S. pyogenes <SEQ ID 595>. The sequence encodes the amino acid sequence <SEQ ID 596>, which is related to SEQ ID 460 of WO02/34771. The encoded polypeptide is a two-component response regulator.

### Example 299

A DNA sequence was identified in S. pyogenes <SEQ ID 597>. The sequence encodes the amino acid sequence <SEQ ID 598>, which is related to SEQ ID 8128 of WO02/34771.

### Example 300

A DNA sequence was identified in S. pyogenes <SEQ ID 599>. The sequence encodes the amino acid sequence <SEQ ID 600>, which is related to SEQ ID 3466 of WO02/34771.

### Example 301

A DNA sequence was identified in S. pyogenes <SEQ ID 601>. The sequence encodes the amino acid sequence <SEQ ID 602>, which is related to SEQ ID 6250 of WO02/34771. The encoded polypeptide is a deoxyribose-phosphate aldolase.

### Example 302

A DNA sequence was identified in S. pyogenes <SEQ ID 603>. The sequence encodes the amino acid sequence <SEQ ID 604>, which is related to SEQ ID 2120 of WO02/34771. The encoded polypeptide is an ABC transporter (ATP-binding protein).

### Example 303

A DNA sequence was identified in S. pyogenes <SEQ ID 605>. The sequence encodes the amino acid sequence <SEQ ID 606>, which is related to SEQ ID 4118 of WO02/34771.

### Example 304

A DNA sequence was identified in S. pyogenes <SEQ ID 607>. The sequence encodes the amino acid sequence <SEQ ID 608>, which is related to SEQ ID 4546 of WO02/34771. The encoded polypeptide is a possible transcriptional regulator.

### Example 305

A DNA sequence was identified in S. pyogenes <SEQ ID 609>. The sequence encodes the amino acid sequence <SEQ ID 610>, which is related to SEQ ID 3700 of WO02/34771. The encoded polypeptide is a ferrichrome ABC transporter (permease).

### Example 306

A DNA sequence was identified in S. pyogenes <SEQ ID 611>. The sequence encodes the amino acid sequence <SEQ ID 612>, which is related to SEQ ID 40. of WO02/34771. The encoded polypeptide is a transcriptional regulator (LysR family).

### Example 307

A DNA sequence was identified in S. pyogenes <SEQ ID 613>. The sequence encodes the amino acid sequence <SEQ ID 614>, which is related to SEQ ID 5708 of WO02/34771.

### Example 308

A DNA sequence was identified in S. pyogenes <SEQ ID 615>. The sequence encodes the amino acid sequence <SEQ ID 616>, which is related to SEQ ID 4790 of WO02/34771.

### Example 309

A DNA sequence was identified in S. pyogenes <SEQ ID 617>. The sequence encodes the amino acid sequence <SEQ ID 618>, which is related to SEQ ID 106 of WO02/34771. The encoded polypeptide is a phosphate starvation-induced protein.

### Example 310

A DNA sequence was identified in S. pyogenes <SEQ ID 619>. The sequence encodes the amino acid sequence <SEQ ID 620>, which is related to SEQ ID 7488 of WO02/34771.

### Example 311

A DNA sequence was identified in S. pyogenes <SEQ ID 621>. The sequence encodes the amino acid sequence <SEQ ID 622>, which is related to SEQ ID 6784 of WO02/34771. The encoded polypeptide is a multi-drug resistance efflux pump.

### Example 312

A DNA sequence was identified in S. pyogenes <SEQ ID 623>. The sequence encodes the amino acid sequence <SEQ ID 624>, which is related to SEQ ID 7550 of WO02/34771.

### Example 313

A DNA sequence was identified in S. pyogenes <SEQ ID 625>. The sequence encodes the amino acid sequence <SEQ ID 626>, which is related to SEQ ID 5490 of WO02/34771.

### Example 314

A DNA sequence was identified in S. pyogenes <SEQ ID 627>. The sequence encodes the amino acid sequence <SEQ ID 628>, which is related to SEQ ID 5494 of WO02/34771. The encoded polypeptide is a peptide chain release factor 2.

### Example 315

A DNA sequence was identified in S. pyogenes <SEQ ID 629>. The sequence encodes the amino acid sequence <SEQ ID 630>, which is related to SEQ ID 1884 of WO02/34771. The encoded polypeptide is a chorismate synthase.

### Example 316

A DNA sequence was identified in S. pyogenes <SEQ ID 631>. The sequence encodes the amino acid sequence <SEQ ID 632>, which is related to SEQ ID 6080 of WO02/34771. The encoded polypeptide is a thioredoxin reductase.

### Example 317

A DNA sequence was identified in S. pyogenes <SEQ ID 633>. The sequence encodes the amino acid sequence <SEQ ID 634>, which is related to SEQ ID 5988 of WO02/34771. The encoded polypeptide is a mevalonate pyrophosphate decarboxylase.

### Example 318

A DNA sequence was identified in S. pyogenes <SEQ ID 635>. The sequence encodes the amino acid sequence <SEQ ID 636>, which is related to SEQ ID 9190 of WO02/34771. The encoded polypeptide is a collagen-like protein.

### Example 319

A DNA sequence was identified in S. pyogenes <SEQ ID 637>. The sequence encodes the amino acid sequence <SEQ ID 638>, which is related to SEQ ID 1292 of WO02/34771. The encoded polypeptide is a spermidine/putrescine ABC transporter (ATP-binding protein).

### Example 320

A DNA sequence was identified in S. pyogenes <SEQ ID 639>. The sequence encodes the amino acid sequence <SEQ ID 640>, which is related to SEQ ID 7942 of WO02/34771. The encoded polypeptide is a decarboxylase, beta subunit.

### Example 321

A DNA sequence was identified in S. pyogenes <SEQ ID 641>. The sequence encodes the amino acid sequence <SEQ ID 642>, which is related to SEQ ID 6192 of WO02/34771.

### Example 322

A DNA sequence was identified in S. pyogenes <SEQ ID 643>. The sequence encodes the amino acid sequence <SEQ ID 644>, which is related to SEQ ID 6648 of WO02/34771. The encoded polypeptide is a transcription factor.

### Example 323

A DNA sequence was identified in S. pyogenes <SEQ ID 645>. The sequence encodes the amino acid sequence <SEQ ID 646>, which is related to SEQ ID 306 of WO02/34771.

### Example 324

A DNA sequence was identified in S. pyogenes <SEQ ID 647>. The sequence encodes the amino acid sequence <SEQ ID 648>, which is related to SEQ ID 3498 of WO02/34771. The encoded polypeptide is an esterase.

### Example 325

A DNA sequence was identified in S. pyogenes <SEQ ID 649>. The sequence encodes the amino acid sequence <SEQ ID 650>, which is related to SEQ ID 6870 of WO02/34771. The encoded polypeptide is a heat shock transcription repressor protein.

### Example 326

A DNA sequence was identified in S. pyogenes <SEQ ID 651>. The sequence encodes the amino acid sequence <SEQ ID 652>, which is related to SEQ ID 3598 of WO02/34771. The encoded polypeptide is a sucrose operon repressor.

### Example 327

A DNA sequence was identified in S. pyogenes <SEQ ID 653>. The sequence encodes the amino acid sequence <SEQ ID 654>, which is related to SEQ ID 6920 of WO02/34771. The encoded polypeptide is a tagatose 6-phosphate kinase.

### Example 328

A DNA sequence was identified in S. pyogenes <SEQ ID 655>. The sequence encodes the amino acid sequence <SEQ ID 656>, which is related to SEQ ID 64. of WO02/34771. The encoded polypeptide is a transmembrane transport protein.

### Example 329

A DNA sequence was identified in S. pyogenes <SEQ ID 657>. The sequence encodes the amino acid sequence <SEQ ID 658>, which is related to SEQ ID 3354 of WO02/34771. The encoded polypeptide is a recombination protein.

### Example 330

A DNA sequence was identified in S. pyogenes <SEQ ID 659>. The sequence encodes the amino acid sequence <SEQ ID 660>, which is related to SEQ ID 914 of WO02/34771. The encoded polypeptide is a phosphoribosylamine-glycine ligase.

### Example 331

A DNA sequence was identified in S. pyogenes <SEQ ID 661>. The sequence encodes the amino acid sequence <SEQ ID 662>, which is related to SEQ ID 2138 of WO02/34771. The encoded polypeptide is a glucose-inhibited division protein.

### Example 332

A DNA sequence was identified in S. pyogenes <SEQ ID 663>. The sequence encodes the amino acid sequence <SEQ ID 664>, which is related to SEQ ID 8198 of WO02/34771.

### Example 333

A DNA sequence was identified in S. pyogenes <SEQ ID 665>. The sequence encodes the amino acid sequence <SEQ ID 666>, which is related to SEQ ID 60. of WO02/34771. The encoded polypeptide is a surface lipoprotein.

### Example 334

A DNA sequence was identified in S. pyogenes <SEQ ID 667>. The sequence encodes the amino acid sequence <SEQ ID 668>, which is related to SEQ ID 2248 of WO02/34771. The encoded polypeptide is a hyaluronate synthase.

### Example 335

A DNA sequence was identified in S. pyogenes <SEQ ID 669>. The sequence encodes the amino acid sequence <SEQ ID 670>, which is related to SEQ ID 3750 of WO02/34771. The encoded polypeptide is a regulatory protein.

### Example 336

A DNA sequence was identified in S. pyogenes <SEQ ID 671>. The sequence encodes the amino acid sequence <SEQ ID 672>, which is related to SEQ ID 9224 of WO02/34771. The encoded polypeptide is a V-type Na⁺-ATPase alpha subunit.

### Example 337

A DNA sequence was identified in S. pyogenes <SEQ ID 673>. The sequence encodes the amino acid sequence <SEQ ID 674>, which is related to SEQ ID 4638 of WO02/34771. The encoded polypeptide is a glycine-betaine binding permease protein.

### Example 338

A DNA sequence was identified in S. pyogenes <SEQ ID 675>. The sequence encodes the amino acid sequence <SEQ ID 676>, which is related to SEQ ID 7340 of WO02/34771.

### Example 339

A DNA sequence was identified in S. pyogenes <SEQ ID 677>. The sequence encodes the amino acid sequence <SEQ ID 678>, which is related to SEQ ID 9184 of WO02/34771. The encoded polypeptide is a regulatory protein—RofA related.

### Example 340

A DNA sequence was identified in S. pyogenes <SEQ ID 679>. The sequence encodes the amino acid sequence <SEQ ID 680>, which is related to SEQ ID 5780 of WO02/34771. The encoded polypeptide is an aminotransferase.

### Example 341

A DNA sequence was identified in S. pyogenes <SEQ ID 681>. The sequence encodes the amino acid sequence <SEQ ID 682>, which is related to SEQ ID 4806 of WO02/34771. The encoded polypeptide is an aminodeoxychorismate lyase.

### Example 342

A DNA sequence was identified in S. pyogenes <SEQ ID 683>. The sequence encodes the amino acid sequence <SEQ ID 684>, which is related to SEQ ID 7646 of WO02/34771. The encoded polypeptide may be phage-associated.

### Example 343

A DNA sequence was identified in S. pyogenes <SEQ ID 685>. The sequence encodes the amino acid sequence <SEQ ID 686>, which is related to SEQ ID 4340 of WO02/34771. The encoded polypeptide is a proton-translocating ATPase, alpha subunit.

### Example 344

A DNA sequence was identified in S. pyogenes <SEQ ID 687>. The sequence encodes the amino acid sequence <SEQ ID 688>, which is related to SEQ ID 4322 of WO02/34771. The encoded polypeptide is a uDP-N-acetylglucosamine 1-carboxyvinyltransferase.

### Example 345

A DNA sequence was identified in S. pyogenes <SEQ ID 689>. The sequence encodes the amino acid sequence <SEQ ID 690>, which is related to SEQ ID 5432 of WO02/34771. The encoded polypeptide is a uracil permease.

### Example 346

A DNA sequence was identified in S. pyogenes <SEQ ID 691>. The sequence encodes the amino acid sequence <SEQ ID 692>, which is related to SEQ ID 2820 of WO02/34771. The encoded polypeptide is a DNA topoisomerase IV (subunit B).

### Example 347

A DNA sequence was identified in S. pyogenes <SEQ ID 693>. The sequence encodes the amino acid sequence <SEQ ID 694>, which is related to SEQ ID 7926 of WO02/34771. The encoded polypeptide is a transcarboxylase subunit.

### Example 348

A DNA sequence was identified in S. pyogenes <SEQ ID 695>. The sequence encodes the amino acid sequence <SEQ ID 696>, which is related to SEQ ID 4460 of WO02/34771. The encoded polypeptide is a deacetylase.

### Example 349

A DNA sequence was identified in S. pyogenes <SEQ ID 697>. The sequence encodes the amino acid sequence <SEQ ID 698>, which is related to SEQ ID 4464 of WO02/34771. The encoded polypeptide is a NADP-dependent glyceraldehyde-3-phosphate dehydrogenase.

### Example 350

A DNA sequence was identified in S. pyogenes <SEQ ID 699>. The sequence encodes the amino acid sequence <SEQ ID 700>, which is related to SEQ ID 6734 of WO02/34771. The encoded polypeptide is an aTP-dependent RNA helicase.

### Example 351

A DNA sequence was identified in S. pyogenes <SEQ ID 701>. The sequence encodes the amino acid sequence <SEQ ID 702>, which is related to SEQ ID 2502 of WO02/34771.

### Example 352

A DNA sequence was identified in S. pyogenes <SEQ ID 703>. The sequence encodes the amino acid sequence <SEQ ID 704>, which is related to SEQ ID 3018 of WO02/34771. The encoded polypeptide is a RNA-binding Sun protein.

### Example 353

A DNA sequence was identified in S. pyogenes <SEQ ID 705>. The sequence encodes the amino acid sequence <SEQ ID 706>, which is related to SEQ ID 2972 of WO02/34771.

### Example 354

A DNA sequence was identified in S. pyogenes <SEQ ID 707>. The sequence encodes the amino acid sequence <SEQ ID 708>, which is related to SEQ ID 3654 of WO02/34771.

### Example 355

A DNA sequence was identified in S. pyogenes <SEQ ID 709>. The sequence encodes the amino acid sequence <SEQ ID 710>, which is related to SEQ ID 5636 of WO02/34771.

### Example 356

A DNA sequence was identified in S. pyogenes <SEQ ID 711>. The sequence encodes the amino acid sequence <SEQ ID 712>, which is related to SEQ ID 1342 of WO02/34771. The encoded polypeptide is a histidine kinase.

### Example 357

A DNA sequence was identified in S. pyogenes <SEQ ID 713>. The sequence encodes the amino acid sequence <SEQ ID 714>, which is related to SEQ ID 6256 of WO02/34771.

### Example 358

A DNA sequence was identified in S. pyogenes <SEQ ID 715>. The sequence encodes the amino acid sequence <SEQ ID 716>, which is related to SEQ ID 3376 of WO02/34771.

### Example 359

A DNA sequence was identified in S. pyogenes <SEQ ID 717>. The sequence encodes the amino acid sequence <SEQ ID 718>, which is related to SEQ ID 8406 of WO02/34771. The encoded polypeptide is a DNA primase—phage associated.

### Example 360

A DNA sequence was identified in S. pyogenes <SEQ ID 719>. The sequence encodes the amino acid sequence <SEQ ID 720>, which is related to SEQ ID 5090 of WO02/34771. The encoded polypeptide is an ABC transporter, ATP-binding protein.

### Example 361

A DNA sequence was identified in S. pyogenes <SEQ ID 721>. The sequence encodes the amino acid sequence <SEQ ID 722>, which is related to SEQ ID 4292 of WO02/34771. The encoded polypeptide is an aTP-dependent exonuclease, subunit A.

### Example 362

A DNA sequence was identified in S. pyogenes <SEQ ID 723>. The sequence encodes the amino acid sequence <SEQ ID 724>, which is related to SEQ ID 380 of WO02/34771. The encoded polypeptide is a DNA-dependent RNA polymerase subunit beta.

### Example 363

A DNA sequence was identified in S. pyogenes <SEQ ID 725>. The sequence encodes the amino acid sequence <SEQ ID 726>, which is related to SEQ ID 384 of WO02/34771. The encoded polypeptide is a DNA-dependent RNA polymerase, B′ subunit.

### Example 364

A DNA sequence was identified in S. pyogenes <SEQ ID 727>. The sequence encodes the amino acid sequence <SEQ ID 728>, which is related to SEQ ID 6298 of WO02/34771. The encoded polypeptide is a cell envelope proteinase.

### Example 365

A DNA sequence was identified in S. pyogenes <SEQ ID 729>. The sequence encodes the amino acid sequence <SEQ ID 730>, which is related to SEQ ID 1040 of WO02/34771. The encoded polypeptide is a phosphoenolpyruvate carboxylase.

### Example 366

A DNA sequence was identified in S. pyogenes <SEQ ID 731>. The sequence encodes the amino acid sequence <SEQ ID 732>, which is related to SEQ ID 4172 of WO02/34771. The encoded polypeptide is a calcium-transporting ATPase.

### Example 367

A DNA sequence was identified in S. pyogenes <SEQ ID 733>. The sequence encodes the amino acid sequence <SEQ ID 734>, which is related to SEQ ID 9100 of WO02/34771. The encoded polypeptide is an extracellular hyaluronate lyase.

### Example 368

A DNA sequence was identified in S. pyogenes <SEQ ID 735>. The sequence encodes the amino acid sequence <SEQ ID 736>, which is related to SEQ ID 1918 of WO02/34771. The encoded polypeptide is a DNA topoisomerase 1.

### Example 369

A DNA sequence was identified in S. pyogenes <SEQ ID 737>. The sequence encodes the amino acid sequence <SEQ ID 738>, which is related to SEQ ID 2950 of WO02/34771. The encoded polypeptide is a penicillin-binding protein 1a.

### Example 370

A DNA sequence was identified in S. pyogenes <SEQ ID 739>. The sequence encodes the amino acid sequence <SEQ ID 740>, which is related to SEQ ID 3490 of WO02/34771. The encoded polypeptide is an initiation factor 2.

### Example 371

A DNA sequence was identified in S. pyogenes <SEQ ID 741>. The sequence encodes the amino acid sequence <SEQ ID 742>, which is related to SEQ ID 1590 of WO02/34771.

### Example 372

A DNA sequence was identified in S. pyogenes <SEQ ID 743>. The sequence encodes the amino acid sequence <SEQ ID 744>, which is related to SEQ ID 822 of WO02/34771. The encoded polypeptide is a C5A peptidase precursor.

### Example 373

A DNA sequence was identified in S. pyogenes <SEQ ID 745>. The sequence encodes the amino acid sequence <SEQ ID 746>, which is related to SEQ ID 5136 of WO02/34771. The encoded polypeptide is a peptidyl-tRNA hydrolase.

### Example 374

A DNA sequence was identified in S. pyogenes <SEQ ID 747>. The sequence encodes the amino acid sequence <SEQ ID 748>, which is related to SEQ ID 9080 of WO02/34771. The encoded polypeptide is an amino acid permease.

### Example 375

A DNA sequence was identified in S. pyogenes <SEQ ID 749>. The sequence encodes the amino acid sequence <SEQ ID 750>, which is related to SEQ ID 1010 of WO02/34771. The encoded polypeptide is a ribose-phosphate pyrophosphokinase.

### Example 376

A DNA sequence was identified in S. pyogenes <SEQ ID 751>. The sequence encodes the amino acid sequence <SEQ ID 752>, which is related to SEQ ID 974 of WO02/34771. The encoded polypeptide is a phosphoribosylpyrophosphate amidotransferase.

### Example 377

A DNA sequence was identified in S. pyogenes <SEQ ID 753>. The sequence encodes the amino acid sequence <SEQ ID 754>, which is related to SEQ ID 910 of WO02/34771. The encoded polypeptide is a phosphoribosylaminoimidazole carboxylase I.

### Example 378

A DNA sequence was identified in S. pyogenes <SEQ ID 755>. The sequence encodes the amino acid sequence <SEQ ID 756>, which is related to SEQ ID 890 of WO02/34771. The encoded polypeptide is a Holliday junction DNA helicase, subunit B.

### Example 379

A DNA sequence was identified in S. pyogenes <SEQ ID 757>. The sequence encodes the amino acid sequence <SEQ ID 758>, which is related to SEQ ID 6422 of WO02/34771. The encoded polypeptide is an alcohol dehydrogenase I.

### Example 380

A DNA sequence was identified in S. pyogenes <SEQ ID 759>. The sequence encodes the amino acid sequence <SEQ ID 760>, which is related to SEQ ID 3988 of WO02/34771. The encoded polypeptide is a preprotein translocase.

### Example 381

A DNA sequence was identified in S. pyogenes <SEQ ID 761>. The sequence encodes the amino acid sequence <SEQ ID 762>, which is related to SEQ ID 364 of WO02/34771. The encoded polypeptide is an ABC transporter (permease).

### Example 382

A DNA sequence was identified in S. pyogenes <SEQ ID 763>. The sequence encodes the amino acid sequence <SEQ ID 764>, which is related to SEQ ID 396 of WO02/34771. The encoded polypeptide is a competence protein, ABC transporter subunit.

### Example 383

A DNA sequence was identified in S. pyogenes <SEQ ID 765>. The sequence encodes the amino acid sequence <SEQ ID 766>, which is related to SEQ ID 404 of WO02/34771.

### Example 384

A DNA sequence was identified in S. pyogenes <SEQ ID 767>. The sequence encodes the amino acid sequence <SEQ ID 768>, which is related to SEQ ID 430 of WO02/34771.

### Example 385

A DNA sequence was identified in S. pyogenes <SEQ ID 769>. The sequence encodes the amino acid sequence <SEQ ID 770>, which is related to SEQ ID 7282 of WO02/34771.

### Example 386

A DNA sequence was identified in S. pyogenes <SEQ ID 771>. The sequence encodes the amino acid sequence <SEQ ID 772>, which is related to SEQ ID 7292 of WO02/34771. The encoded polypeptide is a short-chain fatty acids transporter.

### Example 387

A DNA sequence was identified in S. pyogenes <SEQ ID 773>. The sequence encodes the amino acid sequence <SEQ ID 774>, which is related to SEQ ID 7296 of WO02/34771. The encoded polypeptide is an acetyl-CoA:acetoacetyl-CoA transferase alpha subunit.

### Example 388

A DNA sequence was identified in S. pyogenes <SEQ ID 775>. The sequence encodes the amino acid sequence <SEQ ID 776>, which is related to SEQ ID 7302 of WO02/34771.

### Example 389

A DNA sequence was identified in S. pyogenes <SEQ ID 777>. The sequence encodes the amino acid sequence <SEQ ID 778>, which is related to SEQ ID 7306 of WO02/34771. The encoded polypeptide is a V-type Na⁺-ATPase subunit K.

### Example 390

A DNA sequence was identified in S. pyogenes <SEQ ID 779>. The sequence encodes the amino acid sequence <SEQ ID 780>, which is related to SEQ ID 7316 of WO02/34771. The encoded polypeptide is a V-type Na⁺-ATPase subunit D.

### Example 391

A DNA sequence was identified in S. pyogenes <SEQ ID 781>. The sequence encodes the amino acid sequence <SEQ ID 782>, which is related to SEQ ID 9062 of WO02/34771. The encoded polypeptide is an ABC transporter (lipoprotein).

### Example 392

A DNA sequence was identified in S. pyogenes <SEQ ID 783>. The sequence encodes the amino acid sequence <SEQ ID 784>, which is related to SEQ ID 6270 of WO02/34771. The encoded polypeptide is a transcription antitermination factor.

### Example 393

A DNA sequence was identified in S. pyogenes <SEQ ID 785>. The sequence encodes the amino acid sequence <SEQ ID 786>, which is related to SEQ ID 7322 of WO02/34771.

### Example 394

A DNA sequence was identified in S. pyogenes <SEQ ID 787>. The sequence encodes the amino acid sequence <SEQ ID 788>, which is related to SEQ ID 7324 of WO02/34771. The encoded polypeptide is a streptolysin 0 precursor.

### Example 395

A DNA sequence was identified in S. pyogenes <SEQ ID 789>. The sequence encodes the amino acid sequence <SEQ ID 790>, which is related to SEQ ID 7332 of WO02/34771. The encoded polypeptide is a cystathionine beta-lyase.

### Example 396

A DNA sequence was identified in S. pyogenes <SEQ ID 791>. The sequence encodes the amino acid sequence <SEQ ID 792>, which is related to SEQ ID 4580 of WO02/34771.

### Example 397

A DNA sequence was identified in S. pyogenes <SEQ ID 793>. The sequence encodes the amino acid sequence <SEQ ID 794>, which is related to SEQ ID 4596 of WO02/34771. The encoded polypeptide is a hexylose-6-phosphate isomerase.

### Example 398

A DNA sequence was identified in S. pyogenes <SEQ ID 795>. The sequence encodes the amino acid sequence <SEQ ID 796>, which is related to SEQ ID 4624 of WO02/34771. The encoded polypeptide is a transcriptional regulator.

### Example 399

A DNA sequence was identified in S. pyogenes <SEQ ID 797>. The sequence encodes the amino acid sequence <SEQ ID 798>, which is related to SEQ ID 7350 of WO02/34771. The encoded polypeptide is a nicotinate-nucleotide pyrophosphorylase.

### Example 400

A DNA sequence was identified in S. pyogenes <SEQ ID 799>. The sequence encodes the amino acid sequence <SEQ ID 800>, which is related to SEQ ID 344 herein.

### Example 401

A DNA sequence was identified in S. pyogenes <SEQ ID 801>. The sequence encodes the amino acid sequence <SEQ ID 802>, which is related to SEQ ID 9186 of WO02/34771. The encoded polypeptide is a uDP-glucose pyrophosphorylase.

### Example 402

A DNA sequence was identified in S. pyogenes <SEQ ID 803>. The sequence encodes the amino acid sequence <SEQ ID 804>, which is related to SEQ ID 332 herein.

### Example 403

A DNA sequence was identified in S. pyogenes <SEQ ID 805>. The sequence encodes the amino acid sequence <SEQ ID 806>, which is related to SEQ ID 160 of WO02/34771. The encoded polypeptide is an ABC transporter (ATP-binding protein).

### Example 404

A DNA sequence was identified in S. pyogenes <SEQ ID 807>. The sequence encodes the amino acid sequence <SEQ ID 808>, which is related to SEQ ID 6430 of WO02/34771.

### Example 405

A DNA sequence was identified in S. pyogenes <SEQ ID 809>. The sequence encodes the amino acid sequence <SEQ ID 810>, which is related to SEQ ID 6470 of WO02/34771.

### Example 406

A DNA sequence was identified in S. pyogenes <SEQ ID 811>. The sequence encodes the amino acid sequence <SEQ ID 812>, which is related to SEQ ID 7370 of WO02/34771.

### Example 407

A DNA sequence was identified in S. pyogenes <SEQ ID 813>. The sequence encodes the amino acid sequence <SEQ ID 814>, which is related to SEQ ID 942 of WO02/34771. The encoded polypeptide is a sugar transport system (permease).

### Example 408

A DNA sequence was identified in S. pyogenes <SEQ ID 815>. The sequence encodes the amino acid sequence <SEQ ID 816>, which is related to SEQ ID 5724 of WO02/34771.

### Example 409

A DNA sequence was identified in S. pyogenes <SEQ ID 817>. The sequence encodes the amino acid sequence <SEQ ID 818>, which is related to SEQ ID 5714 of WO02/34771. The encoded polypeptide is a dimethyladenosine transferase.

### Example 410

A DNA sequence was identified in S. pyogenes <SEQ ID 819>. The sequence encodes the amino acid sequence <SEQ ID 820>, which is related to SEQ ID 2158 of WO02/34771. The encoded polypeptide is an amino acid ABC transporter (ATP-binding protein).

### Example 411

A DNA sequence was identified in S. pyogenes <SEQ ID 821>. The sequence encodes the amino acid sequence <SEQ ID 822>, which is related to SEQ ID 1194 of WO02/34771. The encoded polypeptide is a glutamine-binding periplasmic protein.

### Example 412

A DNA sequence was identified in S. pyogenes <SEQ ID 823>. The sequence encodes the amino acid sequence <SEQ ID 824>, which is related to SEQ ID 5796 of WO02/34771. The encoded polypeptide is a negative regulator of genetic competence.

### Example 413

A DNA sequence was identified in S. pyogenes <SEQ ID 825>. The sequence encodes the amino acid sequence <SEQ ID 826>, which is related to SEQ ID 9070 of WO02/34771. The encoded polypeptide is an oligopeptidepermease.

### Example 414

A DNA sequence was identified in S. pyogenes <SEQ ID 827>. The sequence encodes the amino acid sequence <SEQ ID 828>, which is related to SEQ ID 7072 of WO02/34771. The encoded polypeptide is a transposase, IS1548.

### Example 415

A DNA sequence was identified in S. pyogenes <SEQ ID 829>. The sequence encodes the amino acid sequence <SEQ ID 830>, which is related to SEQ ID 5614 of WO02/34771.

### Example 416

A DNA sequence was identified in S. pyogenes <SEQ ID 831>. The sequence encodes the amino acid sequence <SEQ ID 832>, which is related to SEQ ID 7402 of WO02/34771.

### Example 417

A DNA sequence was identified in S. pyogenes <SEQ ID 833>. The sequence encodes the amino acid sequence <SEQ ID 834>, which is related to SEQ ID 2132 of WO02/34771.

### Example 418

A DNA sequence was identified in S. pyogenes <SEQ ID 835>. The sequence encodes the amino acid sequence <SEQ ID 836>, which is related to SEQ ID 4786 of WO02/34771. The encoded polypeptide is an acylphosphatase.

### Example 419

A DNA sequence was identified in S. pyogenes <SEQ ID 837>. The sequence encodes the amino acid sequence <SEQ ID 838>, which is related to SEQ ID 4752 of WO02/34771.

### Example 420

A DNA sequence was identified in S. pyogenes <SEQ ID 839>. The sequence encodes the amino acid sequence <SEQ ID 840>, which is related to SEQ ID 4728 of WO02/34771.

### Example 421

A DNA sequence was identified in S. pyogenes <SEQ ID 841>. The sequence encodes the amino acid sequence <SEQ ID 842>, which is related to SEQ ID 4724 of WO02/34771.

### Example 422

A DNA sequence was identified in S. pyogenes <SEQ ID 843>. The sequence encodes the amino acid sequence <SEQ ID 844>, which is related to SEQ ID 6224 of WO02/34771. The encoded polypeptide is a rRNA methylase.

### Example 423

A DNA sequence was identified in S. pyogenes <SEQ ID 845>. The sequence encodes the amino acid sequence <SEQ ID 846>, which is related to SEQ ID 3728 of WO02/34771.

### Example 424

A DNA sequence was identified in S. pyogenes <SEQ ID 847>. The sequence encodes the amino acid sequence <SEQ ID 848>, which is related to SEQ ID 3720 of WO02/34771. The encoded polypeptide is a hemolysin.

### Example 425

A DNA sequence was identified in S. pyogenes <SEQ ID 849>. The sequence encodes the amino acid sequence <SEQ ID 850>, which is related to SEQ ID 3716 of WO02/34771. The encoded polypeptide is a pyruvate-formate lyase activating enzyme.

### Example 426

A DNA sequence was identified in S. pyogenes <SEQ ID 851>. The sequence encodes the amino acid sequence <SEQ ID 852>, which is related to SEQ ID 2172 of WO02/34771. The encoded polypeptide is a thymidylate kinase.

### Example 427

A DNA sequence was identified in S. pyogenes <SEQ ID 853>. The sequence encodes the amino acid sequence <SEQ ID 854>, which is related to SEQ ID 7424 of WO02/34771.

### Example 428

A DNA sequence was identified in S. pyogenes <SEQ ID 855>. The sequence encodes the amino acid sequence <SEQ ID 856>, which is related to SEQ ID 7456 of WO02/34771.

### Example 429

A DNA sequence was identified in S. pyogenes <SEQ ID 857>. The sequence encodes the amino acid sequence <SEQ ID 858>, which is related to SEQ ID 2256 of WO02/34771.

### Example 430

A DNA sequence was identified in S. pyogenes <SEQ ID 859>. The sequence encodes the amino acid sequence <SEQ ID 860>, which is related to SEQ ID 6446 of WO02/34771. The encoded polypeptide is a glycerol-3-phosphate transporter.

### Example 431

A DNA sequence was identified in S. pyogenes <SEQ ID 861>. The sequence encodes the amino acid sequence <SEQ ID 862>, which is related to SEQ ID 2282 of WO02/34771.

### Example 432

A DNA sequence was identified in S. pyogenes <SEQ ID 863>. The sequence encodes the amino acid sequence <SEQ ID 864>, which is related to SEQ ID 3178 of WO02/34771. The encoded polypeptide is a metal binding protein of ABC transporter (lipoprotein).

### Example 433

A DNA sequence was identified in S. pyogenes <SEQ ID 865>. The sequence encodes the amino acid sequence <SEQ ID 866>, which is related to SEQ ID 8766 of WO02/34771. The encoded polypeptide is an ABC transporter (ATP-binding protein).

### Example 434

A DNA sequence was identified in S. pyogenes <SEQ ID 867>. The sequence encodes the amino acid sequence <SEQ ID 868>, which is related to SEQ ID 52. of WO02/34771. The encoded polypeptide is a 50S ribosomal protein L11.

### Example 435

A DNA sequence was identified in S. pyogenes <SEQ ID 869>. The sequence encodes the amino acid sequence <SEQ ID 870>, which is related to SEQ ID 98. of WO02/34771.

### Example 436

A DNA sequence was identified in S. pyogenes <SEQ ID 871>. The sequence encodes the amino acid sequence <SEQ ID 872>, which is related to SEQ ID 120 of WO02/34771.

### Example 437

A DNA sequence was identified in S. pyogenes <SEQ ID 873>. The sequence encodes the amino acid sequence <SEQ ID 874>, which is related to SEQ ID 112 of WO02/34771.

### Example 438

A DNA sequence was identified in S. pyogenes <SEQ ID 875>. The sequence encodes the amino acid sequence <SEQ ID 876>, which is related to SEQ ID 628 of WO02/34771. The encoded polypeptide is a positive transcriptional regulator.

### Example 439

A DNA sequence was identified in S. pyogenes <SEQ ID 877>. The sequence encodes the amino acid sequence <SEQ ID 878>, which is related to SEQ ID 7516 of WO02/34771.

### Example 440

A DNA sequence was identified in S. pyogenes <SEQ ID 879>. The sequence encodes the amino acid sequence <SEQ ID 880>, which is related to SEQ ID 3878 of WO02/34771.

### Example 441

A DNA sequence was identified in S. pyogenes <SEQ ID 881>. The sequence encodes the amino acid sequence <SEQ ID 882>, which is related to SEQ ID 1206 of WO02/34771.

### Example 442

A DNA sequence was identified in S. pyogenes <SEQ ID 883>. The sequence encodes the amino acid sequence <SEQ ID 884>, which is related to SEQ ID 334 herein. The encoded polypeptide is a positive regulator.

### Example 443

A DNA sequence was identified in S. pyogenes <SEQ ID 885>. The sequence encodes the amino acid sequence <SEQ ID 886>, which is related to SEQ ID 7528 of WO02/34771.

### Example 444

A DNA sequence was identified in S. pyogenes <SEQ ID 887>. The sequence encodes the amino acid sequence <SEQ ID 888>, which is related to SEQ ID 1336 of WO02/34771. The encoded polypeptide is a transposase.

### Example 445

A DNA sequence was identified in S. pyogenes <SEQ ID 889>. The sequence encodes the amino acid sequence <SEQ ID 890>, which is related to SEQ ID 1118 of WO02/34771. The encoded polypeptide is a Hpr kinase/phosphatase.

### Example 446

A DNA sequence was identified in S. pyogenes <SEQ ID 891>. The sequence encodes the amino acid sequence <SEQ ID 892>, which is related to SEQ ID 1074 of WO02/34771. The encoded polypeptide is a lysyl-tRNA synthetase.

### Example 447

A DNA sequence was identified in S. pyogenes <SEQ ID 893>. The sequence encodes the amino acid sequence <SEQ ID 894>, which is related to SEQ ID 7586 of WO02/34771. The encoded polypeptide is a glutathione peroxidase.

### Example 448

A DNA sequence was identified in S. pyogenes <SEQ ID 895>. The sequence encodes the amino acid sequence <SEQ ID 896>, which is related to SEQ ID 1044 of WO02/34771. The encoded polypeptide is an oligopeptidase.

### Example 449

A DNA sequence was identified in S. pyogenes <SEQ ID 897>. The sequence encodes the amino acid sequence <SEQ ID 898>, which is related to SEQ ID 1022 of WO02/34771. The encoded polypeptide is a translation elongation factor EF-Tu.

### Example 450

A DNA sequence was identified in S. pyogenes <SEQ ID 899>. The sequence encodes the amino acid sequence <SEQ ID 900>, which is related to SEQ ID 5464 of WO02/34771. The encoded polypeptide is a peptidoglycan branched peptide synthesis protein, serine/alanine adding enzyme.

### Example 451

A DNA sequence was identified in S. pyogenes <SEQ ID 901>. The sequence encodes the amino acid sequence <SEQ ID 902>, which is related to SEQ ID 2366 of WO02/34771. The encoded polypeptide is a transcriptional regulator (LacI family).

### Example 452

A DNA sequence was identified in S. pyogenes <SEQ ID 903>. The sequence encodes the amino acid sequence <SEQ ID 904>, which is related to SEQ ID 2362 of WO02/34771.

### Example 453

A DNA sequence was identified in S. pyogenes <SEQ ID 905>. The sequence encodes the amino acid sequence <SEQ ID 906>, which is related to SEQ ID 2358 of WO02/34771. The encoded polypeptide is a PTS dependent galactosamine IID component.

### Example 454

A DNA sequence was identified in S. pyogenes <SEQ ID 907>. The sequence encodes the amino acid sequence <SEQ ID 908>, which is related to SEQ ID 1264 of WO02/34771. The encoded polypeptide is a 2-keto-3-deoxygluconate kinase.

### Example 455

A DNA sequence was identified in S. pyogenes <SEQ ID 909>. The sequence encodes the amino acid sequence <SEQ ID 910>, which is related to SEQ ID 5502 of WO02/34771. The encoded polypeptide is a cell-division protein.

### Example 456

A DNA sequence was identified in S. pyogenes <SEQ ID 911>. The sequence encodes the amino acid sequence <SEQ ID 912>, which is related to SEQ ID 7606 of WO02/34771. The encoded polypeptide is a Cro-like protein, phage associated.

### Example 457

A DNA sequence was identified in S. pyogenes <SEQ ID 913>. The sequence encodes the amino acid sequence <SEQ ID 914>, which is related to SEQ ID 7614 of WO02/34771. The encoded polypeptide may be phage-associated.

### Example 458

A DNA sequence was identified in S. pyogenes <SEQ ID 915>. The sequence encodes the amino acid sequence <SEQ ID 916>, which is related to SEQ ID 336 herein. The encoded polypeptide is a DEAD box family helicase, phage associated.

### Example 459

A DNA sequence was identified in S. pyogenes <SEQ ID 917>. The sequence encodes the amino acid sequence <SEQ ID 918>, which is related to SEQ ID 7630 of WO02/34771. The encoded polypeptide may be phage-associated.

### Example 460

A DNA sequence was identified in S. pyogenes <SEQ ID 919>. The sequence encodes the amino acid sequence <SEQ ID 920>, which is related to SEQ ID 7652 of WO02/34771. The encoded polypeptide may be phage-associated.

### Example 461

A DNA sequence was identified in S. pyogenes <SEQ ID 921>. The sequence encodes the amino acid sequence <SEQ ID 922>, which is related to SEQ ID 7662 of WO02/34771. The encoded polypeptide may be phage-associated.

### Example 462

A DNA sequence was identified in S. pyogenes <SEQ ID 923>. The sequence encodes the amino acid sequence <SEQ ID 924>, which is related to SEQ ID 338 herein. The encoded polypeptide may be phage-associated.

### Example 463

A DNA sequence was identified in S. pyogenes <SEQ ID 925>. The sequence encodes the amino acid sequence <SEQ ID 926>, which is related to SEQ ID 340 herein. The encoded polypeptide is a transcriptional regulator (GntR family).

### Example 464

A DNA sequence was identified in S. pyogenes <SEQ ID 927>. The sequence encodes the amino acid sequence <SEQ ID 928>, which is related to SEQ ID 1562 of WO02/34771. The encoded polypeptide is a transposase.

### Example 465

A DNA sequence was identified in S. pyogenes <SEQ ID 929>. The sequence encodes the amino acid sequence <SEQ ID 930>, which is related to SEQ ID 1322 of WO02/34771.

### Example 466

A DNA sequence was identified in S. pyogenes <SEQ ID 931>. The sequence encodes the amino acid sequence <SEQ ID 932>, which is related to SEQ ID 598 of WO02/34771. The encoded polypeptide is an extracellular matrix binding protein.

### Example 467

A DNA sequence was identified in S. pyogenes <SEQ ID 933>. The sequence encodes the amino acid sequence <SEQ ID 934>, which is related to SEQ ID 4304 of WO02/34771. The encoded polypeptide is a phenylalanyl-tRNA synthetase (beta subunit).

### Example 468

A DNA sequence was identified in S. pyogenes <SEQ ID 935>. The sequence encodes the amino acid sequence <SEQ ID 936>, which is related to SEQ ID 6500 of WO02/34771. The encoded polypeptide is an ABC transporter (ATP-binding protein).

### Example 469

A DNA sequence was identified in S. pyogenes <SEQ ID 937>. The sequence encodes the amino acid sequence <SEQ ID 938>, which is related to SEQ ID 3790 of WO02/34771. The encoded polypeptide is a dTDP-4-keto-L-rhamnose reductase.

### Example 470

A DNA sequence was identified in S. pyogenes <SEQ ID 939>. The sequence encodes the amino acid sequence <SEQ ID 940>, which is related to SEQ ID 7724 of WO02/34771. The encoded polypeptide is an ABC-transporter (permease protein)—possibly involved in cell wall localization and side chain formation of rhamnose-glucose polysaccharide.

### Example 471

A DNA sequence was identified in S. pyogenes <SEQ ID 941>. The sequence encodes the amino acid sequence <SEQ ID 942>, which is related to SEQ ID 7732 of WO02/34771.

### Example 472

A DNA sequence was identified in S. pyogenes <SEQ ID 943>. The sequence encodes the amino acid sequence <SEQ ID 944>, which is related to SEQ ID 3678 of WO02/34771. The encoded polypeptide is a tripeptidase.

### Example 473

A DNA sequence was identified in S. pyogenes <SEQ ID 945>. The sequence encodes the amino acid sequence <SEQ ID 946>, which is related to SEQ ID 1902 of WO02/34771.

### Example 474

A DNA sequence was identified in S. pyogenes <SEQ ID 947>. The sequence encodes the amino acid sequence <SEQ ID 948>, which is related to SEQ ID 1876 of WO02/34771. The encoded polypeptide is a glutathione reductase (GR).

### Example 475

A DNA sequence was identified in S. pyogenes <SEQ ID 949>. The sequence encodes the amino acid sequence <SEQ ID 950>, which is related to SEQ ID 342 herein. The encoded polypeptide is a folyl-polyglutamate synthetase.

### Example 476

A DNA sequence was identified in S. pyogenes <SEQ ID 951>. The sequence encodes the amino acid sequence <SEQ ID 952>, which is related to SEQ ID 2034 of WO02/34771. The encoded polypeptide is an aspartate transcarbamoylase.

### Example 477

A DNA sequence was identified in S. pyogenes <SEQ ID 953>. The sequence encodes the amino acid sequence <SEQ ID 954>, which is related to SEQ ID 2026 of WO02/34771. The encoded polypeptide is a carbamoylphosphate synthetase.

### Example 478

A DNA sequence was identified in S. pyogenes <SEQ ID 955>. The sequence encodes the amino acid sequence <SEQ ID 956>, which is related to SEQ ID 2530 of WO02/34771. The encoded polypeptide is an ABC transporter (ATP-binding protein).

### Example 479

A DNA sequence was identified in S. pyogenes <SEQ ID 957>. The sequence encodes the amino acid sequence <SEQ ID 958>, which is related to SEQ ID 2574 of WO02/34771. The encoded polypeptide is a glycerophosphodiester phosphodiesterase.

### Example 480

A DNA sequence was identified in S. pyogenes <SEQ ID 959>. The sequence encodes the amino acid sequence <SEQ ID 960>, which is related to SEQ ID 770 of WO02/34771.

### Example 481

A DNA sequence was identified in S. pyogenes <SEQ ID 961>. The sequence encodes the amino acid sequence <SEQ ID 962>, which is related to SEQ ID 6038 of WO02/34771. The encoded polypeptide is a poly(A) polymerase.

### Example 482

A DNA sequence was identified in S. pyogenes <SEQ ID 963>. The sequence encodes the amino acid sequence <SEQ ID 964>, which is related to SEQ ID 5982 of WO02/34771. The encoded polypeptide is a phosphomevalonate kinase.

### Example 483

A DNA sequence was identified in S. pyogenes <SEQ ID 965>. The sequence encodes the amino acid sequence <SEQ ID 966>, which is related to SEQ ID 5978 of WO02/34771.

### Example 484

A DNA sequence was identified in S. pyogenes <SEQ ID 967>. The sequence encodes the amino acid sequence <SEQ ID 968>, which is related to SEQ ID 5958 of WO02/34771. The encoded polypeptide is a 3-hydroxy-3-methylglutaryl-coenzyme A synthase (1-IMG-CoA synthase).

### Example 485

A DNA sequence was identified in S. pyogenes <SEQ ID 969>. The sequence encodes the amino acid sequence <SEQ ID 970>, which is related to SEQ ID 5954 of WO02/34771. The encoded polypeptide is a thymidylate synthase.

### Example 486

A DNA sequence was identified in S. pyogenes <SEQ ID 971>. The sequence encodes the amino acid sequence <SEQ ID 972>, which is related to SEQ ID 9078 of WO02/34771. The encoded polypeptide is an arsenate reductase.

### Example 487

A DNA sequence was identified in S. pyogenes <SEQ ID 973>. The sequence encodes the amino acid sequence <SEQ ID 974>, which is related to SEQ ID 2756 of WO02/34771. The encoded polypeptide is a histidine protein kinase.

### Example 488

A DNA sequence was identified in S. pyogenes <SEQ ID 975>. The sequence encodes the amino acid sequence <SEQ ID 976>, which is related to SEQ ID 2814 of WO02/34771.

### Example 489

A DNA sequence was identified in S. pyogenes <SEQ ID 977>. The sequence encodes the amino acid sequence <SEQ ID 978>, which is related to SEQ ID 2824 of WO02/34771. The encoded polypeptide is a DNA topoisomerase IV (subunit C).

### Example 490

A DNA sequence was identified in S. pyogenes <SEQ ID 979>. The sequence encodes the amino acid sequence <SEQ ID 980>, which is related to SEQ ID 2828 of WO02/34771. The encoded polypeptide is a branched-chain-amino-acid aminotransferase.

### Example 491

A DNA sequence was identified in S. pyogenes <SEQ ID 981>. The sequence encodes the amino acid sequence <SEQ ID 982>, which is related to SEQ ID 2832 of WO02/34771.

### Example 492

A DNA sequence was identified in S. pyogenes <SEQ ID 983>. The sequence encodes the amino acid sequence <SEQ ID 984>, which is related to SEQ ID 2604 of WO02/34771. The encoded polypeptide is an exfoliative toxin.

### Example 493

A DNA sequence was identified in S. pyogenes <SEQ ID 985>. The sequence encodes the amino acid sequence <SEQ ID 986>, which is related to SEQ ID 2616 of WO02/34771. The encoded polypeptide is a GTP-binding protein.

### Example 494

A DNA sequence was identified in S. pyogenes <SEQ ID 987>. The sequence encodes the amino acid sequence <SEQ ID 988>, which is related to SEQ ID 2620 of WO02/34771.

### Example 495

A DNA sequence was identified in S. pyogenes <SEQ ID 989>. The sequence encodes the amino acid sequence <SEQ ID 990>, which is related to SEQ ID 2652 of WO02/34771.

### Example 496

A DNA sequence was identified in S. pyogenes <SEQ ID 991>. The sequence encodes the amino acid sequence <SEQ ID 992>, which is related to SEQ ID 7784 of WO02/34771.

### Example 497

A DNA sequence was identified in S. pyogenes <SEQ ID 993>. The sequence encodes the amino acid sequence <SEQ ID 994>, which is related to SEQ ID 7786 of WO02/34771.

### Example 498

A DNA sequence was identified in S. pyogenes <SEQ ID 995>. The sequence encodes the amino acid sequence <SEQ ID 996>, which is related to SEQ ID 7794 of WO02/34771.

### Example 499

A DNA sequence was identified in S. pyogenes <SEQ ID 997>. The sequence encodes the amino acid sequence <SEQ ID 998>, which is related to SEQ ID 7812 of WO02/34771.

### Example 500

A DNA sequence was identified in S. pyogenes <SEQ ID 999>. The sequence encodes the amino acid sequence <SEQ ID 1000>, which is related to SEQ ID 7826 of WO02/34771. The encoded polypeptide may be phage-associated.

### Example 501

A DNA sequence was identified in S. pyogenes <SEQ ID 1001>. The sequence encodes the amino acid sequence <SEQ ID 1002>, which is related to SEQ ID 7828 of WO02/34771. The encoded polypeptide is a terminase, small subunit—phage associated.

### Example 502

A DNA sequence was identified in S. pyogenes <SEQ ID 1003>. The sequence encodes the amino acid sequence <SEQ ID 1004>, which is related to SEQ ID 7830 of WO02/34771. The encoded polypeptide is a terminase, large subunit—phage associated.

### Example 503

A DNA sequence was identified in S. pyogenes <SEQ ID 1005>. The sequence encodes the amino acid sequence <SEQ ID 1006>, which is related to SEQ ID 7842 of WO02/34771.

### Example 504

A DNA sequence was identified in S. pyogenes <SEQ ID 1007>. The sequence encodes the amino acid sequence <SEQ ID 1008>, which is related to SEQ ID 7846 of WO02/34771. The encoded polypeptide may be phage-associated.

### Example 505

A DNA sequence was identified in S. pyogenes <SEQ ID 1009>. The sequence encodes the amino acid sequence <SEQ ID 1010>, which is related to SEQ ID 7854 of WO02/34771. The encoded polypeptide may be phage-associated.

### Example 506

A DNA sequence was identified in S. pyogenes <SEQ ID 1011>. The sequence encodes the amino acid sequence <SEQ ID 1012>, which is related to SEQ ID 7890 of WO02/34771. The encoded polypeptide is a streptococcal exotoxin I.

### Example 507

A DNA sequence was identified in S. pyogenes <SEQ ID 1013>. The sequence encodes the amino acid sequence <SEQ ID 1014>, which is related to SEQ ID 2700 of WO02/34771.

### Example 508

A DNA sequence was identified in S. pyogenes <SEQ ID 1015>. The sequence encodes the amino acid sequence <SEQ ID 1016>, which is related to SEQ ID 2708 of WO02/34771. The encoded polypeptide is a fibronectin-binding protein-like protein A.

### Example 509

A DNA sequence was identified in S. pyogenes <SEQ ID 1017>. The sequence encodes the amino acid sequence <SEQ ID 1018>, which is related to SEQ ID 7898 of WO02/34771.

### Example 510

A DNA sequence was identified in S. pyogenes <SEQ ID 1019>. The sequence encodes the amino acid sequence <SEQ ID 1020>, which is related to SEQ ID 2716 of WO02/34771. The encoded polypeptide is an ABC transport protein (permease).

### Example 511

A DNA sequence was identified in S. pyogenes <SEQ ID 1021>. The sequence encodes the amino acid sequence <SEQ ID 1022>, which is related to SEQ ID 4276 of WO02/34771. The encoded polypeptide is an acetoin dehydrogenase (TPP-dependent) alpha chain.

### Example 512

A DNA sequence was identified in S. pyogenes <SEQ ID 1023>. The sequence encodes the amino acid sequence <SEQ ID 1024>, which is related to SEQ ID 4254 of WO02/34771. The encoded polypeptide is a uDP-N-acetylmuramyl tripeptide synthetase.

### Example 513

A DNA sequence was identified in S. pyogenes <SEQ ID 1025>. The sequence encodes the amino acid sequence <SEQ ID 1026>, which is related to SEQ ID 4228 of WO02/34771. The encoded polypeptide is a coproporphyrinogen III oxidase.

### Example 514

A DNA sequence was identified in S. pyogenes <SEQ ID 1027>. The sequence encodes the amino acid sequence <SEQ ID 1028>, which is related to SEQ ID 4200 of WO02/34771.

### Example 515

A DNA sequence was identified in S. pyogenes <SEQ ID 1029>. The sequence encodes the amino acid sequence <SEQ ID 1030>, which is related to SEQ ID 1636 of WO02/34771. The encoded polypeptide is a phosphotransferase system (PTS), enzyme II, component C.

### Example 516

A DNA sequence was identified in S. pyogenes <SEQ ID 1031>. The sequence encodes the amino acid sequence <SEQ ID 1032>, which is related to SEQ ID 1618 of WO02/34771. The encoded polypeptide is a periplasmic-iron-binding protein.

### Example 517

A DNA sequence was identified in S. pyogenes <SEQ ID 1033>. The sequence encodes the amino acid sequence <SEQ ID 1034>, which is related to SEQ ID 5366 of WO02/34771. The encoded polypeptide is a succinic semialdehyde dehydrogenase.

### Example 518

A DNA sequence was identified in S. pyogenes <SEQ ID 1035>. The sequence encodes the amino acid sequence <SEQ ID 1036>, which is related to SEQ ID 2588 of WO02/34771. The encoded polypeptide is a dipeptidase.

### Example 519

A DNA sequence was identified in S. pyogenes <SEQ ID 1037>. The sequence encodes the amino acid sequence <SEQ ID 1038>, which is related to SEQ ID 5918 of WO02/34771. The encoded polypeptide is a 505 ribosomal protein L₁₀.

### Example 520

A DNA sequence was identified in S. pyogenes <SEQ ID 1039>. The sequence encodes the amino acid sequence <SEQ ID 1040>, which is related to SEQ ID 5914 of WO02/34771. The encoded polypeptide is a 505 ribosomal protein L₇/L₁₂.

### Example 521

A DNA sequence was identified in S. pyogenes <SEQ ID 1041>. The sequence encodes the amino acid sequence <SEQ ID 1042>, which is related to SEQ ID 2436 of WO02/34771. The encoded polypeptide is a methyl transferase.

### Example 522

A DNA sequence was identified in S. pyogenes <SEQ ID 1043>. The sequence encodes the amino acid sequence <SEQ ID 1044>, which is related to SEQ ID 1832 of WO02/34771. The encoded polypeptide is an ABC transporter (ATP-binding)—lantibiotic associated.

### Example 523

A DNA sequence was identified in S. pyogenes <SEQ ID 1045>. The sequence encodes the amino acid sequence <SEQ ID 1046>, which is related to SEQ ID 5342 of WO02/34771. The encoded polypeptide is a folyl-polyglutamate synthetase.

### Example 524

A DNA sequence was identified in S. pyogenes <SEQ ID 1047>. The sequence encodes the amino acid sequence <SEQ ID 1048>, which is related to SEQ ID 5316 of WO02/34771. The encoded polypeptide is a spermidine/putrescine ABC transporter (permease protein).

### Example 525

A DNA sequence was identified in S. pyogenes <SEQ ID 1049>. The sequence encodes the amino acid sequence <SEQ ID 1050>, which is related to SEQ ID 5304 of WO02/34771.

### Example 526

A DNA sequence was identified in S. pyogenes <SEQ ID 1051>. The sequence encodes the amino acid sequence <SEQ ID 1052>, which is related to SEQ ID 5252 of WO02/34771.

### Example 527

A DNA sequence was identified in S. pyogenes <SEQ ID 1053>. The sequence encodes the amino acid sequence <SEQ ID 1054>, which is related to SEQ ID 636 of WO02/34771. The encoded polypeptide is an ABC transporter (binding protein).

### Example 528

A DNA sequence was identified in S. pyogenes <SEQ ID 1055>. The sequence encodes the amino acid sequence <SEQ ID 1056>, which is related to SEQ ID 6618 of WO02/34771. The encoded polypeptide is a GMP reductase.

### Example 529

A DNA sequence was identified in S. pyogenes <SEQ ID 1057>. The sequence encodes the amino acid sequence <SEQ ID 1058>, which is related to SEQ ID 6566 of WO02/34771.

### Example 530

A DNA sequence was identified in S. pyogenes <SEQ ID 1059>. The sequence encodes the amino acid sequence <SEQ ID 1060>, which is related to SEQ ID 6562 of WO02/34771. The encoded polypeptide is a serine hydroxymethyltransferase.

### Example 531

A DNA sequence was identified in S. pyogenes <SEQ ID 1061>. The sequence encodes the amino acid sequence <SEQ ID 1062>, which is related to SEQ ID 4650 of WO02/34771.

### Example 532

A DNA sequence was identified in S. pyogenes <SEQ ID 1063>. The sequence encodes the amino acid sequence <SEQ ID 1064>, which is related to SEQ ID 7920 of WO02/34771.

### Example 533

A DNA sequence was identified in S. pyogenes <SEQ ID 1065>. The sequence encodes the amino acid sequence <SEQ ID 1066>, which is related to SEQ ID 124 of WO02/34771. The encoded polypeptide is a D-specific D-2-hydroxyacid dehydrogenase.

### Example 534

A DNA sequence was identified in S. pyogenes <SEQ ID 1067>. The sequence encodes the amino acid sequence <SEQ ID 1068>, which is related to SEQ ID 7936 of WO02/34771. The encoded polypeptide is a Mg2+/citrate complex transporter.

### Example 535

A DNA sequence was identified in S. pyogenes <SEQ ID 1069>. The sequence encodes the amino acid sequence <SEQ ID 1070>, which is related to SEQ ID 7952 of WO02/34771.

### Example 536

A DNA sequence was identified in S. pyogenes <SEQ ID 1071>. The sequence encodes the amino acid sequence <SEQ ID 1072>, which is related to SEQ ID 7954 of WO02/34771. The encoded polypeptide is an oxaloacetate decarboxylase alpha chain.

### Example 537

A DNA sequence was identified in S. pyogenes <SEQ ID 1073>. The sequence encodes the amino acid sequence <SEQ ID 1074>, which is related to SEQ ID 6516 of WO02/34771. The encoded polypeptide is a repressor protein.

### Example 538

A DNA sequence was identified in S. pyogenes <SEQ ID 1075>. The sequence encodes the amino acid sequence <SEQ ID 1076>, which is related to SEQ ID 2090 of WO02/34771. The encoded polypeptide is a signal recognition particle.

### Example 539

A DNA sequence was identified in S. pyogenes <SEQ ID 1077>. The sequence encodes the amino acid sequence <SEQ ID 1078>, which is related to SEQ ID 2052 of WO02/34771. The encoded polypeptide is an ABC transporter (ATP-binding protein).

### Example 540

A DNA sequence was identified in S. pyogenes <SEQ ID 1079>. The sequence encodes the amino acid sequence <SEQ ID 1080>, which is related to SEQ ID 6168 of WO02/34771. The encoded polypeptide is a flavoprotein.

### Example 541

A DNA sequence was identified in S. pyogenes <SEQ ID 1081>. The sequence encodes the amino acid sequence <SEQ ID 1082>, which is related to SEQ ID 6160 of WO02/34771.

### Example 542

A DNA sequence was identified in S. pyogenes <SEQ ID 1083>. The sequence encodes the amino acid sequence <SEQ ID 1084>, which is related to SEQ ID 4672 of WO02/34771. The encoded polypeptide is a sugar ABC transporter (permease protein).

### Example 543

A DNA sequence was identified in S. pyogenes <SEQ ID 1085>. The sequence encodes the amino acid sequence <SEQ ID 1086>, which is related to SEQ ID 4678 of WO02/34771. The encoded polypeptide is a sugar ABC transporter (ATP-binding protein).

### Example 544

A DNA sequence was identified in S. pyogenes <SEQ ID 1087>. The sequence encodes the amino acid sequence <SEQ ID 1088>, which is related to SEQ ID 4688 of WO02/34771.

### Example 545

A DNA sequence was identified in S. pyogenes <SEQ ID 1089>. The sequence encodes the amino acid sequence <SEQ ID 1090>, which is related to SEQ ID 2074 of WO02/34771. The encoded polypeptide is a lysyl-aminopeptidase; aminopeptidase N.

### Example 546

A DNA sequence was identified in S. pyogenes <SEQ ID 1091>. The sequence encodes the amino acid sequence <SEQ ID 1092>, which is related to SEQ ID 5828 of WO02/34771.

### Example 547

A DNA sequence was identified in S. pyogenes <SEQ ID 1093>. The sequence encodes the amino acid sequence <SEQ ID 1094>, which is related to SEQ ID 1354 of WO02/34771. The encoded polypeptide is an ABC transporter (ATP-binding protein).

### Example 548

A DNA sequence was identified in S. pyogenes <SEQ ID 1095>. The sequence encodes the amino acid sequence <SEQ ID 1096>, which is related to SEQ ID 5082 of WO02/34771. The encoded polypeptide is a maltose operon transcriptional repressor.

### Example 549

A DNA sequence was identified in S. pyogenes <SEQ ID 1097>. The sequence encodes the amino acid sequence <SEQ ID 1098>, which is related to SEQ ID 310 herein. The encoded polypeptide is a cyclomaltodextrinase.

### Example 550

A DNA sequence was identified in S. pyogenes <SEQ ID 1099>. The sequence encodes the amino acid sequence <SEQ ID 1100>, which is related to SEQ ID 314 herein. The encoded polypeptide is a beta-glucosidase.

### Example 551

A DNA sequence was identified in S. pyogenes <SEQ ID 1101>. The sequence encodes the amino acid sequence <SEQ ID 1102>, which is related to SEQ ID 7996 of WO02/34771.

### Example 552

A DNA sequence was identified in S. pyogenes <SEQ ID 1103>. The sequence encodes the amino acid sequence <SEQ ID 1104>, which is related to SEQ ID 4032 of WO02/34771.

### Example 553

A DNA sequence was identified in S. pyogenes <SEQ ID 1105>. The sequence encodes the amino acid sequence <SEQ ID 1106>, which is related to SEQ ID 4394 of WO02/34771.

### Example 554

A DNA sequence was identified in S. pyogenes <SEQ ID 1107>. The sequence encodes the amino acid sequence <SEQ ID 1108>, which is related to SEQ ID 4454 of WO02/34771. The encoded polypeptide is a RNA helicase.

### Example 555

A DNA sequence was identified in S. pyogenes <SEQ ID 1109>. The sequence encodes the amino acid sequence <SEQ ID 1110>, which is related to SEQ ID 4476 of WO02/34771. The encoded polypeptide is a glutaredoxin.

### Example 556

A DNA sequence was identified in S. pyogenes <SEQ ID 1111>. The sequence encodes the amino acid sequence <SEQ ID 1112>, which is related to SEQ ID 412 of WO02/34771. The encoded polypeptide is a transcriptional regulator protein.

### Example 557

A DNA sequence was identified in S. pyogenes <SEQ ID 1113>. The sequence encodes the amino acid sequence <SEQ ID 1114>, which is related to SEQ ID 6602 of WO02/34771. The encoded polypeptide is a formate dehydrogenase.

### Example 558

A DNA sequence was identified in S. pyogenes <SEQ ID 1115>. The sequence encodes the amino acid sequence <SEQ ID 1116>, which is related to SEQ ID 5456 of WO02/34771. The encoded polypeptide is a dihydroorotate dehydrogenase.

### Example 559

A DNA sequence was identified in S. pyogenes <SEQ ID 1117>. The sequence encodes the amino acid sequence <SEQ ID 1118>, which is related to SEQ ID 8036 of WO02/34771.

### Example 560

A DNA sequence was identified in S. pyogenes <SEQ ID 1119>. The sequence encodes the amino acid sequence <SEQ ID 1120>, which is related to SEQ ID 1420 of WO02/34771. The encoded polypeptide is a structural protein—phage associated.

### Example 561

A DNA sequence was identified in S. pyogenes <SEQ ID 1121>. The sequence encodes the amino acid sequence <SEQ ID 1122>, which is related to SEQ ID 1444 of WO02/34771. The encoded polypeptide may be phage-associated.

### Example 562

A DNA sequence was identified in S. pyogenes <SEQ ID 1123>. The sequence encodes the amino acid sequence <SEQ ID 1124>, which is related to SEQ ID 8058 of WO02/34771. The encoded polypeptide may be phage-associated.

### Example 563

A DNA sequence was identified in S. pyogenes <SEQ ID 1125>. The sequence encodes the amino acid sequence <SEQ ID 1126>, which is related to SEQ ID 1546 of WO02/34771. The encoded polypeptide is a repressor—phage associated.

### Example 564

A DNA sequence was identified in S. pyogenes <SEQ ID 1127>. The sequence encodes the amino acid sequence <SEQ ID 1128>, which is related to SEQ ID 302 of WO02/34771.

### Example 565

A DNA sequence was identified in S. pyogenes <SEQ ID 1129>. The sequence encodes the amino acid sequence <SEQ ID 1130>, which is related to SEQ ID 298 of WO02/34771. The encoded polypeptide is a DNA repair and genetic recombination protein.

### Example 566

A DNA sequence was identified in S. pyogenes <SEQ ID 1131>. The sequence encodes the amino acid sequence <SEQ ID 1132>, which is related to SEQ ID 292 of WO02/34771. The encoded polypeptide is a repressor protein.

### Example 567

A DNA sequence was identified in S. pyogenes <SEQ ID 1133>. The sequence encodes the amino acid sequence <SEQ ID 1134>, which is related to SEQ ID 276 of WO02/34771. The encoded polypeptide is an exodeoxyribonuclease VII (large subunit).

### Example 568

A DNA sequence was identified in S. pyogenes <SEQ ID 1135>. The sequence encodes the amino acid sequence <SEQ ID 1136>, which is related to SEQ ID 270 of WO02/34771. The encoded polypeptide is a bifunctional methylenetetrahydrofolate dehydrogenase/methenyltetrahydrofolate cyclohydrolase.

### Example 569

A DNA sequence was identified in S. pyogenes <SEQ ID 1137>. The sequence encodes the amino acid sequence <SEQ ID 1138>, which is related to SEQ ID 266 of WO02/34771. The encoded polypeptide is a phosphomannomutase.

### Example 570

A DNA sequence was identified in S. pyogenes <SEQ ID 1139>. The sequence encodes the amino acid sequence <SEQ ID 1140>, which is related to SEQ ID 8092 of WO02/34771. The encoded polypeptide is a deoxyribodipyrimidine photolyase.

### Example 571

A DNA sequence was identified in S. pyogenes <SEQ ID 1141>. The sequence encodes the amino acid sequence <SEQ ID 1142>, which is related to SEQ ID 1186 of WO02/34771. The encoded polypeptide is an amino acid ABC transporter (ATP-binding protein).

### Example 572

A DNA sequence was identified in S. pyogenes <SEQ ID 1143>. The sequence encodes the amino acid sequence <SEQ ID 1144>, which is related to SEQ ID 234 of WO02/34771.

### Example 573

A DNA sequence was identified in S. pyogenes <SEQ ID 1145>. The sequence encodes the amino acid sequence <SEQ ID 1146>, which is related to SEQ ID 222 of WO02/34771. The encoded polypeptide is a cell division protein.

### Example 574

A DNA sequence was identified in S. pyogenes <SEQ ID 1147>. The sequence encodes the amino acid sequence <SEQ ID 1148>, which is related to SEQ ID 194 of WO02/34771.

### Example 575

A DNA sequence was identified in S. pyogenes <SEQ ID 1149>. The sequence encodes the amino acid sequence <SEQ ID 1150>, which is related to SEQ ID 6492 of WO02/34771. The encoded polypeptide is a ribose transport operon repressor.

### Example 576

A DNA sequence was identified in S. pyogenes <SEQ ID 1151>. The sequence encodes the amino acid sequence <SEQ ID 1152>, which is related to SEQ ID 8124 of WO02/34771.

### Example 577

A DNA sequence was identified in S. pyogenes <SEQ ID 1153>. The sequence encodes the amino acid sequence <SEQ ID 1154>, which is related to SEQ ID 728 of WO02/34771.

### Example 578

A DNA sequence was identified in S. pyogenes <SEQ ID 1155>. The sequence encodes the amino acid sequence <SEQ ID 1156>, which is related to SEQ ID 9068 of WO02/34771. The encoded polypeptide is a transposase—IS1548.

### Example 579

A DNA sequence was identified in S. pyogenes <SEQ ID 1157>. The sequence encodes the amino acid sequence <SEQ ID 1158>, which is related to SEQ ID 1034 of WO02/34771.

### Example 580

A DNA sequence was identified in S. pyogenes <SEQ ID 1159>. The sequence encodes the amino acid sequence <SEQ ID 1160>, which is related to SEQ ID 5298 of WO02/34771. The encoded polypeptide is a two-component sensor response regulator.

### Example 581

A DNA sequence was identified in S. pyogenes <SEQ ID 1161>. The sequence encodes the amino acid sequence <SEQ ID 1162>, which is related to SEQ ID 1964 of WO02/34771. The encoded polypeptide is a two-component sensor histidine kinase.

### Example 582

A DNA sequence was identified in S. pyogenes <SEQ ID 1163>. The sequence encodes the amino acid sequence <SEQ ID 1164>, which is related to SEQ ID 8152 of WO02/34771. The encoded polypeptide is a hyaluronidase.

### Example 583

A DNA sequence was identified in S. pyogenes <SEQ ID 1165>. The sequence encodes the amino acid sequence <SEQ ID 1166>, which is related to SEQ ID 2996 of WO02/34771. The encoded polypeptide is a two-component response regulator.

### Example 584

A DNA sequence was identified in S. pyogenes <SEQ ID 1167>. The sequence encodes the amino acid sequence <SEQ ID 1168>, which is related to SEQ ID 3010 of WO02/34771. The encoded polypeptide is a primosomal replication factor Y.

### Example 585

A DNA sequence was identified in S. pyogenes <SEQ ID 1169>. The sequence encodes the amino acid sequence <SEQ ID 1170>, which is related to SEQ ID 3006 of WO02/34771.

### Example 586

A DNA sequence was identified in S. pyogenes <SEQ ID 1171>. The sequence encodes the amino acid sequence <SEQ ID 1172>, which is related to SEQ ID 8172 of WO02/34771. The encoded polypeptide is an acetyl-CoA:acetoacetyl-CoA transferase A subunit.

### Example 587

A DNA sequence was identified in S. pyogenes <SEQ ID 1173>. The sequence encodes the amino acid sequence <SEQ ID 1174>, which is related to SEQ ID 2942 of WO02/34771. The encoded polypeptide is a NAD+ synthase.

### Example 588

A DNA sequence was identified in S. pyogenes <SEQ ID 1175>. The sequence encodes the amino acid sequence <SEQ ID 1176>, which is related to SEQ ID 4070 of WO02/34771. The encoded polypeptide is an amino acid permease.

### Example 589

A DNA sequence was identified in S. pyogenes <SEQ ID 1177>. The sequence encodes the amino acid sequence <SEQ ID 1178>, which is related to SEQ ID 2930 of WO02/34771. The encoded polypeptide is an amino acid ABC transport system (ATP-binding protein).

### Example 590

A DNA sequence was identified in S. pyogenes <SEQ ID 1179>. The sequence encodes the amino acid sequence <SEQ ID 1180>, which is related to SEQ ID 2912 of WO02/34771. The encoded polypeptide is an undecaprenyl-phosphate-UDP-MurNAc-pentapeptide phospho-MurNAc-pentapeptide transferase.

### Example 591

A DNA sequence was identified in S. pyogenes <SEQ ID 1181>. The sequence encodes the amino acid sequence <SEQ ID 1182>, which is related to SEQ ID 2900 of WO02/34771. The encoded polypeptide is a cell division protein.

### Example 592

A DNA sequence was identified in S. pyogenes <SEQ ID 1183>. The sequence encodes the amino acid sequence <SEQ ID 1184>, which is related to SEQ ID 2888 of WO02/34771. The encoded polypeptide is a gamma-glutamyl kinase.

### Example 593

A DNA sequence was identified in S. pyogenes <SEQ ID 1185>. The sequence encodes the amino acid sequence <SEQ ID 1186>, which is related to SEQ ID 2866 of WO02/34771. The encoded polypeptide is a transcriptional regulatory protein.

### Example 594

A DNA sequence was identified in S. pyogenes <SEQ ID 1187>. The sequence encodes the amino acid sequence <SEQ ID 1188>, which is related to SEQ ID 8194 of WO02/34771.

### Example 595

A DNA sequence was identified in S. pyogenes <SEQ ID 1189>. The sequence encodes the amino acid sequence <SEQ ID 1190>, which is related to SEQ ID 780 of WO02/34771. The encoded polypeptide is a reductase/dehydrogenase.

### Example 596

A DNA sequence was identified in S. pyogenes <SEQ ID 1191>. The sequence encodes the amino acid sequence <SEQ ID 1192>, which is related to SEQ ID 320 herein. The encoded polypeptide is a transcription regulator.

### Example 597

A DNA sequence was identified in S. pyogenes <SEQ ID 1193>. The sequence encodes the amino acid sequence <SEQ ID 1194>, which is related to SEQ ID 3518 of WO02/34771.

### Example 598

A DNA sequence was identified in S. pyogenes <SEQ ID 1195>. The sequence encodes the amino acid sequence <SEQ ID 1196>, which is related to SEQ ID 3514 of WO02/34771.

### Example 599

A DNA sequence was identified in S. pyogenes <SEQ ID 1197>. The sequence encodes the amino acid sequence <SEQ ID 1198>, which is related to SEQ ID 6916 of WO02/34771. The encoded polypeptide is a galactose-6-phosphate isomerase.

### Example 600

A DNA sequence was identified in S. pyogenes <SEQ ID 1199>. The sequence encodes the amino acid sequence <SEQ ID 1200>, which is related to SEQ ID 1574 of WO02/34771. The encoded polypeptide is a PTS system, enzyme IIC component.

### Example 601

A DNA sequence was identified in S. pyogenes <SEQ ID 1201>. The sequence encodes the amino acid sequence <SEQ ID 1202>, which is related to SEQ ID 3506 of WO02/34771. The encoded polypeptide is a cation-transporting ATP-ase—copper transport operon.

### Example 602

A DNA sequence was identified in S. pyogenes <SEQ ID 1203>. The sequence encodes the amino acid sequence <SEQ ID 1204>, which is related to SEQ ID 3502 of WO02/34771. The encoded polypeptide is a negative transcriptional regulator—cpooer transport operon.

### Example 603

A DNA sequence was identified in S. pyogenes <SEQ ID 1205>. The sequence encodes the amino acid sequence <SEQ ID 1206>, which is related to SEQ ID 3482 of WO02/34771.

### Example 604

A DNA sequence was identified in S. pyogenes <SEQ ID 1207>. The sequence encodes the amino acid sequence <SEQ ID 1208>, which is related to SEQ ID 3478 of WO02/34771. The encoded polypeptide is a transcription termination-antitermination factor.

### Example 605

A DNA sequence was identified in S. pyogenes <SEQ ID 1209>. The sequence encodes the amino acid sequence <SEQ ID 1210>, which is related to SEQ ID 3474 of WO02/34771.

### Example 606

A DNA sequence was identified in S. pyogenes <SEQ ID 1211>. The sequence encodes the amino acid sequence <SEQ ID 1212>, which is related to SEQ ID 5212 of WO02/34771. The encoded polypeptide is an acetyl-CoA carboxylase biotin carboxylase subunit.

### Example 607

A DNA sequence was identified in S. pyogenes <SEQ ID 1213>. The sequence encodes the amino acid sequence <SEQ ID 1214>, which is related to SEQ ID 5208 of WO02/34771. The encoded polypeptide is a beta-hydroxyacyl-ACP dehydratase.

### Example 608

A DNA sequence was identified in S. pyogenes <SEQ ID 1215>. The sequence encodes the amino acid sequence <SEQ ID 1216>, which is related to SEQ ID 5196 of WO02/34771. The encoded polypeptide is a malonyl CoA-acyl carrier protein transacylase.

### Example 609

A DNA sequence was identified in S. pyogenes <SEQ ID 1217>. The sequence encodes the amino acid sequence <SEQ ID 1218>, which is related to SEQ ID 6882 of WO02/34771. The encoded polypeptide is a heat-shock (chaperone) protein.

### Example 610

A DNA sequence was identified in S. pyogenes <SEQ ID 1219>. The sequence encodes the amino acid sequence <SEQ ID 1220>, which is related to SEQ ID 9234 of WO02/34771.

### Example 611

A DNA sequence was identified in S. pyogenes <SEQ ID 1221>. The sequence encodes the amino acid sequence <SEQ ID 1222>, which is related to SEQ ID 5576 of WO02/34771. The encoded polypeptide is a Glu-tRNA Gln amidotransferase subunit C.

### Example 612

A DNA sequence was identified in S. pyogenes <SEQ ID 1223>. The sequence encodes the amino acid sequence <SEQ ID 1224>, which is related to SEQ ID 5562 of WO02/34771. The encoded polypeptide is a pyrazinamidase/nicotinamidase.

### Example 613

A DNA sequence was identified in S. pyogenes <SEQ ID 1225>. The sequence encodes the amino acid sequence <SEQ ID 1226>, which is related to SEQ ID 3662 of WO02/34771. The encoded polypeptide is an aminotransferase.

### Example 614

A DNA sequence was identified in S. pyogenes <SEQ ID 1227>. The sequence encodes the amino acid sequence <SEQ ID 1228>, which is related to SEQ ID 3658 of WO02/34771.

### Example 615

A DNA sequence was identified in S. pyogenes <SEQ ID 1229>. The sequence encodes the amino acid sequence <SEQ ID 1230>, which is related to SEQ ID 4416 of WO02/34771. The encoded polypeptide is an ABC transporter (ATP-binding protein).

### Example 616

A DNA sequence was identified in S. pyogenes <SEQ ID 1231>. The sequence encodes the amino acid sequence <SEQ ID 1232>, which is related to SEQ ID 8228 of WO02/34771. The encoded polypeptide shares similarity with several eukaryotic proteins.

### Example 617

A DNA sequence was identified in S. pyogenes <SEQ ID 1233>. The sequence encodes the amino acid sequence <SEQ ID 1234>, which is related to SEQ ID 346 herein.

### Example 618

A DNA sequence was identified in S. pyogenes <SEQ ID 1235>. The sequence encodes the amino acid sequence <SEQ ID 1236>, which is related to SEQ ID 3602 of WO02/34771. The encoded polypeptide is a sucrose-6-phosphate hydrolase.

### Example 619

A DNA sequence was identified in S. pyogenes <SEQ ID 1237>. The sequence encodes the amino acid sequence <SEQ ID 1238>, which is related to SEQ ID 3594 of WO02/34771. The encoded polypeptide is a transcriptional terminator.

### Example 620

A DNA sequence was identified in S. pyogenes <SEQ ID 1239>. The sequence encodes the amino acid sequence <SEQ ID 1240>, which is related to SEQ ID 3574 of WO02/34771. The encoded polypeptide is a late competence protein required for DNA binding.

### Example 621

A DNA sequence was identified in S. pyogenes <SEQ ID 1241>. The sequence encodes the amino acid sequence <SEQ ID 1242>, which is related to SEQ ID 3568 of WO02/34771. The encoded polypeptide is an aminopeptidase P; XAA-pro aminopeptidase.

### Example 622

A DNA sequence was identified in S. pyogenes <SEQ ID 1243>. The sequence encodes the amino acid sequence <SEQ ID 1244>, which is related to SEQ ID 3560 of WO02/34771. The encoded polypeptide is an excinuclease ABC (subunit A).

### Example 623

A DNA sequence was identified in S. pyogenes <SEQ ID 1245>. The sequence encodes the amino acid sequence <SEQ ID 1246>, which is related to SEQ ID 5066 of WO02/34771. The encoded polypeptide is an a/G-specific adenine glycosylase.

### Example 624

A DNA sequence was identified in S. pyogenes <SEQ ID 1247>. The sequence encodes the amino acid sequence <SEQ ID 1248>, which is related to SEQ ID 1740 of WO02/34771.

### Example 625

A DNA sequence was identified in S. pyogenes <SEQ ID 1249>. The sequence encodes the amino acid sequence <SEQ ID 1250>, which is related to SEQ ID 5056 of WO02/34771. The encoded polypeptide is a DNA mismatch repair protein.

### Example 626

A DNA sequence was identified in S. pyogenes <SEQ ID 1251>. The sequence encodes the amino acid sequence <SEQ ID 1252>, which is related to SEQ ID 5024 of WO02/34771. The encoded polypeptide is a pyruvate formate-lyase.

### Example 627

A DNA sequence was identified in S. pyogenes <SEQ ID 1253>. The sequence encodes the amino acid sequence <SEQ ID 1254>, which is related to SEQ ID 9178 of WO02/34771.

### Example 628

A DNA sequence was identified in S. pyogenes <SEQ ID 1255>. The sequence encodes the amino acid sequence <SEQ ID 1256>, which is related to SEQ ID 5000 of WO02/34771. The encoded polypeptide is an antibiotic resistance protein NorA.

### Example 629

A DNA sequence was identified in S. pyogenes <SEQ ID 1257>. The sequence encodes the amino acid sequence <SEQ ID 1258>, which is related to SEQ ID 1712 of WO02/34771. The encoded polypeptide is a transcriptional activator regulator protein.

### Example 630

A DNA sequence was identified in S. pyogenes <SEQ ID 1259>. The sequence encodes the amino acid sequence <SEQ ID 1260>, which is related to SEQ ID 6246 of WO02/34771. The encoded polypeptide is a nucleoside transporter.

### Example 631

A DNA sequence was identified in S. pyogenes <SEQ ID 1261>. The sequence encodes the amino acid sequence <SEQ ID 1262>, which is related to SEQ ID 4922 of WO02/34771. The encoded polypeptide is a ribosomal-protein-alanine acetyltransferase.

### Example 632

A DNA sequence was identified in S. pyogenes <SEQ ID 1263>. The sequence encodes the amino acid sequence <SEQ ID 1264>, which is related to SEQ ID 6908 of WO02/34771. The encoded polypeptide is a transcription regulator—(trigger factor (prolyl isomerase)).

### Example 633

A DNA sequence was identified in S. pyogenes <SEQ ID 1265>. The sequence encodes the amino acid sequence <SEQ ID 1266>, which is related to SEQ ID 6680 of WO02/34771.

### Example 634

A DNA sequence was identified in S. pyogenes <SEQ ID 1267>. The sequence encodes the amino acid sequence <SEQ ID 1268>, which is related to SEQ ID 324 herein. The encoded polypeptide is a phospho-beta-D-galactosidase.

### Example 635

A DNA sequence was identified in S. pyogenes <SEQ ID 1269>. The sequence encodes the amino acid sequence <SEQ ID 1270>, which is related to SEQ ID 570 of WO02/34771. The encoded polypeptide is a 50S ribosomal protein L13.

### Example 636

A DNA sequence was identified in S. pyogenes <SEQ ID 1271>. The sequence encodes the amino acid sequence <SEQ ID 1272>, which is related to SEQ ID 556 of WO02/34771. The encoded polypeptide is a tRNA/rRNA methyltransferase.

### Example 637

A DNA sequence was identified in S. pyogenes <SEQ ID 1273>. The sequence encodes the amino acid sequence <SEQ ID 1274>, which is related to SEQ ID 510 of WO02/34771.

### Example 638

A DNA sequence was identified in S. pyogenes <SEQ ID 1275>. The sequence encodes the amino acid sequence <SEQ ID 1276>, which is related to SEQ ID 496 of WO02/34771.

### Example 639

A DNA sequence was identified in S. pyogenes <SEQ ID 1277>. The sequence encodes the amino acid sequence <SEQ ID 1278>, which is related to SEQ ID 2318 of WO02/34771. The encoded polypeptide is a transcriptional regulator (MarR family).

### Example 640

A DNA sequence was identified in S. pyogenes <SEQ ID 1279>. The sequence encodes the amino acid sequence <SEQ ID 1280>, which is related to SEQ ID 6930 of WO02/34771. The encoded polypeptide is a leucine-rich protein.

### Example 641

A DNA sequence was identified in S. pyogenes <SEQ ID 1281>. The sequence encodes the amino acid sequence <SEQ ID 1282>, which is related to SEQ ID 1720 of WO02/34771.

### Example 642

A DNA sequence was identified in S. pyogenes <SEQ ID 1283>. The sequence encodes the amino acid sequence <SEQ ID 1284>, which is related to SEQ ID 28. of WO02/34771. The encoded polypeptide is a para-aminobenzoate synthetase.

### Example 643

A DNA sequence was identified in S. pyogenes <SEQ ID 1285>. The sequence encodes the amino acid sequence <SEQ ID 1286>, which is related to SEQ ID 8302 of WO02/34771. The encoded polypeptide is a pail protein (theoretical repressor).

### Example 644

A DNA sequence was identified in S. pyogenes <SEQ ID 1287>. The sequence encodes the amino acid sequence <SEQ ID 1288>, which is related to SEQ ID 8306 of WO02/34771. The encoded polypeptide is a mitogenic exotoxin Z.

### Example 645

A DNA sequence was identified in S. pyogenes <SEQ ID 1289>. The sequence encodes the amino acid sequence <SEQ ID 1290>, which is related to SEQ ID 9194 of WO02/34771.

### Example 646

A DNA sequence was identified in S. pyogenes <SEQ ID 1291>. The sequence encodes the amino acid sequence <SEQ ID 1292>, which is related to SEQ ID 3064 of WO02/34771. The encoded polypeptide is a PTS system, enzyme BB.

### Example 647

A DNA sequence was identified in S. pyogenes <SEQ ID 1293>. The sequence encodes the amino acid sequence <SEQ ID 1294>, which is related to SEQ ID 3050 of WO02/34771. The encoded polypeptide is a transcriptional regulator.

### Example 648

A DNA sequence was identified in S. pyogenes <SEQ ID 1295>. The sequence encodes the amino acid sequence <SEQ ID 1296>, which is related to SEQ ID 9196 of WO02/34771. The encoded polypeptide is a dipeptidase.

### Example 649

A DNA sequence was identified in S. pyogenes <SEQ ID 1297>. The sequence encodes the amino acid sequence <SEQ ID 1298>, which is related to SEQ ID 6234 of WO02/34771. The encoded polypeptide is a heat shock protein (chaperonin).

### Example 650

A DNA sequence was identified in S. pyogenes <SEQ ID 1299>. The sequence encodes the amino acid sequence <SEQ ID 1300>, which is related to SEQ ID 6230 of WO02/34771. The encoded polypeptide is a heat shock protein—cochaperonin.

### Example 651

A DNA sequence was identified in S. pyogenes <SEQ ID 1301>. The sequence encodes the amino acid sequence <SEQ ID 1302>, which is related to SEQ ID 3326 of WO02/34771. The encoded polypeptide is a cold shock protein.

### Example 652

A DNA sequence was identified in S. pyogenes <SEQ ID 1303>. The sequence encodes the amino acid sequence <SEQ ID 1304>, which is related to SEQ ID 8370 of WO02/34771.

### Example 653

A DNA sequence was identified in S. pyogenes <SEQ ID 1305>. The sequence encodes the amino acid sequence <SEQ ID 1306>, which is related to SEQ ID 8372 of WO02/34771. The encoded polypeptide is a histidine ammonia-lyase.

### Example 654

A DNA sequence was identified in S. pyogenes <SEQ ID 1307>. The sequence encodes the amino acid sequence <SEQ ID 1308>, which is related to SEQ ID 8374 of WO02/34771. The encoded polypeptide is a formiminoglutamate hydrolase.

### Example 655

A DNA sequence was identified in S. pyogenes <SEQ ID 1309>. The sequence encodes the amino acid sequence <SEQ ID 1310>, which is related to SEQ ID 4526 of WO02/34771. The encoded polypeptide is an elongation factor TS.

### Example 656

A DNA sequence was identified in S. pyogenes <SEQ ID 1311>. The sequence encodes the amino acid sequence <SEQ ID 1312>, which is related to SEQ ID 3396 of WO02/34771. The encoded polypeptide is an anaerobic ribonucleotide reductase activator.

### Example 657

A DNA sequence was identified in S. pyogenes <SEQ ID 1313>. The sequence encodes the amino acid sequence <SEQ ID 1314>, which is related to SEQ ID 3388 of WO02/34771. The encoded polypeptide is an oxidoreductase.

### Example 658

A DNA sequence was identified in S. pyogenes <SEQ ID 1315>. The sequence encodes the amino acid sequence <SEQ ID 1316>, which is related to SEQ ID 3346 of WO02/34771. The encoded polypeptide is a 3-methyl-adenine DNA glycosylase I, constitutive.

### Example 659

A DNA sequence was identified in S. pyogenes <SEQ ID 1317>. The sequence encodes the amino acid sequence <SEQ ID 1318>, which is related to SEQ ID 3332 of WO02/34771. The encoded polypeptide is a DNA mismatch repair protein.

### Example 660

A DNA sequence was identified in S. pyogenes <SEQ ID 1319>. The sequence encodes the amino acid sequence <SEQ ID 1320>, which is related to SEQ ID 578 of WO02/34771. The encoded polypeptide is an integrase—phage associated.

### Example 661

A DNA sequence was identified in S. pyogenes <SEQ ID 1321>. The sequence encodes the amino acid sequence <SEQ ID 1322>, which is related to SEQ ID 8392 of WO02/34771.

### Example 662

A DNA sequence was identified in S. pyogenes <SEQ ID 1323>. The sequence encodes the amino acid sequence <SEQ ID 1324>, which is related to SEQ ID 8396 of WO02/34771.

### Example 663

A DNA sequence was identified in S. pyogenes <SEQ ID 1325>. The sequence encodes the amino acid sequence <SEQ ID 1326>, which is related to SEQ ID 8412 of WO02/34771.

### Example 664

A DNA sequence was identified in S. pyogenes <SEQ ID 1327>. The sequence encodes the amino acid sequence <SEQ ID 1328>, which is related to SEQ ID 3310 of WO02/34771.

### Example 665

A DNA sequence was identified in S. pyogenes <SEQ ID 1329>. The sequence encodes the amino acid sequence <SEQ ID 1330>, which is related to SEQ ID 3298 of WO02/34771. The encoded polypeptide is an aspartyl-tRNA synthetase.

### Example 666

A DNA sequence was identified in S. pyogenes <SEQ ID 1331>. The sequence encodes the amino acid sequence <SEQ ID 1332>, which is related to SEQ ID 6152 of WO02/34771. The encoded polypeptide is a cadmium resistance protein.

### Example 667

A DNA sequence was identified in S. pyogenes <SEQ ID 1333>. The sequence encodes the amino acid sequence <SEQ ID 1334>, which is related to SEQ ID 8432 of WO02/34771. The encoded polypeptide is a cadmium efflux system accessory.

### Example 668

A DNA sequence was identified in S. pyogenes <SEQ ID 1335>. The sequence encodes the amino acid sequence <SEQ ID 1336>, which is related to SEQ ID 8442 of WO02/34771.

### Example 669

A DNA sequence was identified in S. pyogenes <SEQ ID 1337>. The sequence encodes the amino acid sequence <SEQ ID 1338>, which is related to SEQ ID 330 herein. The encoded polypeptide is a transposase.

### Example 670

A DNA sequence was identified in S. pyogenes <SEQ ID 1339>. The sequence encodes the amino acid sequence <SEQ ID 1340>, which is related to SEQ ID 3214 of WO02/34771.

### Example 671

A DNA sequence was identified in S. pyogenes <SEQ ID 1341>. The sequence encodes the amino acid sequence <SEQ ID 1342>, which is related to SEQ ID 2424 of WO02/34771. The encoded polypeptide is a DNA polymerase III delta prime subunit.

### Example 672

A DNA sequence was identified in S. pyogenes <SEQ ID 1343>. The sequence encodes the amino acid sequence <SEQ ID 1344>, which is related to SEQ ID 3194 of WO02/34771. The encoded polypeptide is a tRNA-(5-methylaminomethyl-2-thiouridylate).

### Example 673

A DNA sequence was identified in S. pyogenes <SEQ ID 1345>. The sequence encodes the amino acid sequence <SEQ ID 1346>, which is related to SEQ ID 3152 of WO02/34771.

### Example 674

A DNA sequence was identified in S. pyogenes <SEQ ID 1347>. The sequence encodes the amino acid sequence <SEQ ID 1348>, which is related to SEQ ID 3106 of WO02/34771. The encoded polypeptide is a tryptophanyl-tRNA synthetase.

### Example 675

A DNA sequence was identified in S. agalactiae <SEQ ID 1349>. The sequence encodes the amino acid sequence <SEQ ID 1350>, which is related to SEQ ID 980 of WO02/34771. The encoded polypeptide is a phosphoribosylformylglycinamidine synthase.

### Example 676

A DNA sequence was identified in S. agalactiae <SEQ ID 1351>. The sequence encodes the amino acid sequence <SEQ ID 1352>, which is related to SEQ ID 6842 of WO02/34771. The encoded polypeptide is a lipoprotein.

### Example 677

A DNA sequence was identified in S. agalactiae <SEQ ID 1353>. The sequence encodes the amino acid sequence <SEQ ID 1354>, which is related to SEQ ID 8698 of WO02/34771. The encoded polypeptide is an ABC transporter/ATP-binding protein.

### Example 678

A DNA sequence was identified in S. agalactiae <SEQ ID 1355>. The sequence encodes the amino acid sequence <SEQ ID 1356>, which is related to SEQ ID 124 herein.

### Example 679

A DNA sequence was identified in S. agalactiae <SEQ ID 1357>. The sequence encodes the amino acid sequence <SEQ ID 1358>, which is related to SEQ ID 222 herein.

### Example 680

A DNA sequence was identified in S. agalactiae <SEQ ID 1359>. The sequence encodes the amino acid sequence <SEQ ID 1360>, which is related to SEQ ID 5424 of WO02/34771.

### Example 681

A DNA sequence was identified in S. agalactiae <SEQ ID 1361>. The sequence encodes the amino acid sequence <SEQ ID 1362>, which is related to SEQ ID 10570 of WO02/34771. The encoded polypeptide is a transposase (OrfA) of the IS3 family.

### Example 682

A DNA sequence was identified in S. agalactiae <SEQ ID 1363>. The sequence encodes the amino acid sequence <SEQ ID 1364>, which is related to SEQ ID 5968 of WO02/34771. The encoded polypeptide is a hemolysin III.

### Example 683

A DNA sequence was identified in S. agalactiae <SEQ ID 1365>. The sequence encodes the amino acid sequence <SEQ ID 1366>, which is related to SEQ ID 5588 of WO02/34771. The encoded polypeptide is a membrane protein.

### Example 684

A DNA sequence was identified in S. agalactiae <SEQ ID 1367>. The sequence encodes the amino acid sequence <SEQ ID 1368>, which is related to SEQ ID 3656 of WO02/34771.

### Example 685

A DNA sequence was identified in S. agalactiae <SEQ ID 1369>. The sequence encodes the amino acid sequence <SEQ ID 1370>, which is related to SEQ ID 5378 of WO02/34771.

### Example 686

A DNA sequence was identified in S. agalactiae <SEQ ID 1371>. The sequence encodes a conserved amino acid sequence <SEQ ID 1372>.

It will be understood that the invention is described above by way of example only and modifications may be made whilst remaining within the scope and spirit of the invention.

1. An isolated protein comprising:
(a) an amino acid sequence selected from the group consisting of SEQ IDs 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370 & 1372;
(b) an amino acid sequence having 50% or greater sequence identity to a protein of (a); or
(c) a fragment of 7 or more consecutive amino acids of a protein of (a).

2-3. (canceled)

4. An isolated antibody which binds to a protein according claim 1.

5. The antibody of claim 4, wherein said antibody is a monoclonal antibody, a chimeric antibody, a humanised antibody, or a fully human antibody.

6. An isolated nucleic acid molecule which encodes a protein according to claim 1.

7. A nucleic acid molecule according to claim 6, comprising a nucleotide sequence selected from the group consisting of
(a) SEQ IDs 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369 & 1371 or the complement thereof; and
(b) a nucleic acid molecule comprising a fragment of 10 or more consecutive nucleotides from a sequence of (a) or the complement thereof

8-12. (canceled)

13. A composition comprising the protein of claim 1.

14-28. (canceled)

29. A method of inducing an immune response against S. pyogenes or S. agalactiae, comprising administering to an individual in need thereof a composition comprising the protein of claim 1.
